SEQUENCE LISTING (44 sequences)

SEQ ID NO: 1 - DNA, 57 bp, Artificial Sequence
  Primer
gtctcgtgggctcggagatgtgtataagagacagcagggtgtggagcagcctgccaa

SEQ ID NO: 2 - DNA, 57 bp, Artificial Sequence
  Primer
gtctcgtgggctcggagatgtgtataagagacagatctattggtaccgacaggttcc

SEQ ID NO: 3 - DNA, 55 bp, Artificial Sequence
  Primer
gtctcgtgggctcggagatgtgtataagagacagggcgagcaggtggagcagcgc

SEQ ID NO: 4 - DNA, 55 bp, Artificial Sequence
  Primer
gtctcgtgggctcggagatgtgtataagagacagtctgctctgagatgcaatttt

SEQ ID NO: 5 - DNA, 58 bp, Artificial Sequence
  Primer
gtctcgtgggctcggagatgtgtataagagacagctacttcccttggtataagcaaga

SEQ ID NO: 6 - DNA, 56 bp, Artificial Sequence
  Primer
gtctcgtgggctcggagatgtgtataagagacagacccaactctkttctggtatgt

SEQ ID NO: 7 - DNA, 57 bp, Artificial Sequence
  Primer
gtctcgtgggctcggagatgtgtataagagacagaaggtacagcagagcccagaatc

SEQ ID NO: 8 - DNA, 56 bp, Artificial Sequence
  Primer
gtctcgtgggctcggagatgtgtataagagacagcctgagcatccacgagggtgaa

SEQ ID NO: 9 - DNA, 55 bp, Artificial Sequence
  Primer
gtctcgtgggctcggagatgtgtataagagacagagctgagatgcaastattcct

SEQ ID NO: 10 - DNA, 57 bp, Artificial Sequence
  Primer
gtctcgtgggctcggagatgtgtataagagacagcatggagagaaggtcgagcaaca

SEQ ID NO: 11 - DNA, 56 bp, Artificial Sequence
  Primer
gtctcgtgggctcggagatgtgtataagagacagaagacccaagtggagcagagtc

SEQ ID NO: 12 - DNA, 56 bp, Artificial Sequence
  Primer
gtctcgtgggctcggagatgtgtataagagacaggtgacccagacagaaggcctgg

SEQ ID NO: 13 - DNA, 54 bp, Artificial Sequence
  Primer
gtctcgtgggctcggagatgtgtataagagacaggtccttggttctgcaggagg

SEQ ID NO: 14 - DNA, 54 bp, Artificial Sequence
  Primer
gtctcgtgggctcggagatgtgtataagagacagcagcagcaggtgagacaaag

SEQ ID NO: 15 - DNA, 58 bp, Artificial Sequence
  Primer
gtctcgtgggctcggagatgtgtataagagacagctggactgttcatatgagacaagt

SEQ ID NO: 16 - DNA, 56 bp, Artificial Sequence
  Primer
gtctcgtgggctcggagatgtgtataagagacagagaaggtaacacagactcagac

SEQ ID NO: 17 - DNA, 57 bp, Artificial Sequence
  Primer
gtctcgtgggctcggagatgtgtataagagacagcagtccgtggaccagcctgatgc

SEQ ID NO: 18 - DNA, 57 bp, Artificial Sequence
  Primer
gtctcgtgggctcggagatgtgtataagagacaggagcagagtcctcggtttctgag

SEQ ID NO: 19 - DNA, 58 bp, Artificial Sequence
  Primer
gtctcgtgggctcggagatgtgtataagagacagccagcaagttaaacaaagctctcc

SEQ ID NO: 20 - DNA, 55 bp, Artificial Sequence
  Primer
gtctcgtgggctcggagatgtgtataagagacagcctccgtttctcggctcctgg

SEQ ID NO: 21 - DNA, 57 bp, Artificial Sequence
  Primer
gtctcgtgggctcggagatgtgtataagagacaggtgactttgctggagcaaaaccc

SEQ ID NO: 22 - DNA, 57 bp, Artificial Sequence
  Primer
gtctcgtgggctcggagatgtgtataagagacaggacccgaaaattatccagaaacc

SEQ ID NO: 23 - DNA, 57 bp, Artificial Sequence
  Primer
gtctcgtgggctcggagatgtgtataagagacagggacccaaagtcttacagatccc

SEQ ID NO: 24 - DNA, 57 bp, Artificial Sequence
  Primer
gtctcgtgggctcggagatgtgtataagagacaggagacggctgttttccagactcc

SEQ ID NO: 25 - DNA, 57 bp, Artificial Sequence
  Primer
gtctcgtgggctcggagatgtgtataagagacagaacactaaaattactcagtcacc

SEQ ID NO: 26 - DNA, 34 bp, Artificial Sequence
  Primer
gtctcgtgggctcggagatgtgtataagagacag

SEQ ID NO: 27 - DNA, 57 bp, Artificial Sequence
  Primer
gtctcgtgggctcggagatgtgtataagagacaggaggctgcagtcacccaaagccc

SEQ ID NO: 28 - DNA, 57 bp, Artificial Sequence
  Primer
gtctcgtgggctcggagatgtgtataagagacaggaggctgcagtcacccaaagtcc

SEQ ID NO: 29 - DNA, 57 bp, Artificial Sequence
  Primer
gtctcgtgggctcggagatgtgtataagagacaggaagctggagtcacccagtctcc

SEQ ID NO: 30 - DNA, 57 bp, Artificial Sequence
  Primer
gtctcgtgggctcggagatgtgtataagagacaggatgctggagttacccagacacc

SEQ ID NO: 31 - DNA, 57 bp, Artificial Sequence
  Primer
gtctcgtgggctcggagatgtgtataagagacagaatgctggtgtcatccaaacacc

SEQ ID NO: 32 - DNA, 57 bp, Artificial Sequence
  Primer
gtctcgtgggctcggagatgtgtataagagacaggatactacggttaagcagaaccc

SEQ ID NO: 33 - DNA, 57 bp, Artificial Sequence
  Primer
gtctcgtgggctcggagatgtgtataagagacagggtggcatcattactcagacacc

SEQ ID NO: 34 - DNA, 57 bp, Artificial Sequence
  Primer
gtctcgtgggctcggagatgtgtataagagacagggagcactcgtctatcaatatcc

SEQ ID NO: 35 - DNA, 57 bp, Artificial Sequence
  Primer
gtctcgtgggctcggagatgtgtataagagacaggactctggggttgtccagaatcc

SEQ ID NO: 36 - DNA, 57 bp, Artificial Sequence
  Primer
gtctcgtgggctcggagatgtgtataagagacaggatgctgcagttacacagaagcc

SEQ ID NO: 37 - DNA, 57 bp, Artificial Sequence
  Primer
gtctcgtgggctcggagatgtgtataagagacaggttgctggagtaacccagactcc

SEQ ID NO: 38 - DNA, 57 bp, Artificial Sequence
  Primer
gtctcgtgggctcggagatgtgtataagagacagaattcaaaagtcattcagactcc

SEQ ID NO: 39 - DNA, 57 bp, Artificial Sequence
  Primer
gtctcgtgggctcggagatgtgtataagagacaggacatgaaagtaacccagatgcc

SEQ ID NO: 40 - DNA, 57 bp, Artificial Sequence
  Primer
gtctcgtgggctcggagatgtgtataagagacagagtgtcctcctctaccaaaagcc

SEQ ID NO: 41 - DNA, 57 bp, Artificial Sequence
  Primer
gtctcgtgggctcggagatgtgtataagagacaggctcagactatccatcaatggcc

SEQ ID NO: 42 - protein, 450 aa, Unknown
  E54K/L372P Tn5 transposase
MITSALHRAADWAKSVFSSAALGDPRRTARLVNVAAQLAKYSGKSITISSEGSKAMQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDECQLLGYLDKGKRKRKEKAGSLQWAYMAIARLGGFMDSKRTGIASWGALW

SEQ ID NO: 43 - DNA, 66 bp, Artificial Sequence
  Oligo(dT) primer
  misc_feature (23)..(48): n is a, c, g, or t
  misc_feature (33)..(48): Unique spatial barcode
ctacacgacgctcttccgatctnnnnnnnnnnnnnnnnnnnnnnnnnntttttttttttttttttt

SEQ ID NO: 44 - DNA, 70 bp, Artificial Sequence
  Indexing i5 primer
  misc_feature (30)..(37): n is a, c, g, or t
aatgatacggcgaccaccgagatctacacnnnnnnnnacactctttccctacacgacgctcttccgatct

(19) United States
(12) Patent Application Publication
(10) Pub. No.: US 2023/0212656 A1
(43) Pub. Date: Jul. 6, 2023

(54) METHODS OF SPATIALLY RESOLVED SINGLE CELL SEQUENCING

(71) Applicant: THE REGENTS OF THE UNIVERSITY OF CALIFORNIA, Oakland, CA (US)

(72) Inventors: Eric CHOW, San Francisco, CA (US); Alexander MARSON, San Francisco, CA (US); Youjin LEE, San Francisco, CA (US); Derek BOGDANOFF, San Francisco, CA (US); Jonathan WOO, San Francisco, CA (US); Chun Jimmie YE, San Francisco, CA (US)

(73) Assignee: THE REGENTS OF THE UNIVERSITY OF CALIFORNIA, Oakland, CA (US)

(21) Appl. No.: 17/801,517

(22) PCT Filed: Feb. 22, 2021
(86) PCT No.: PCT/US2021/019126

§ 371 Date: Aug. 22, 2022

Related U.S. Application Data
(60) Provisional application No. 62/979,235, filed on Feb. 20, 2020.

(51) Int. Cl. C12Q 1/6841; C12Q 1/6874; C12N 15/10

(52) U.S. Cl. CPC C12Q 1/6841; C12N 15/1065; C12Q 1/6874

(57) ABSTRACT

The present disclosure generally relates to spatial detection of a nucleic acid, such as a genomic DNA or a RNA transcript, in a cell comprised in a tissue sample. The present disclosure provides methods for detecting and/or analyzing nucleic acids, such as chromatin or RNA transcripts, so as to obtain spatial information about the localization, distribution or expression of genes in a tissue sample. The present disclosure thus provides a process for performing “spatial transcriptomics” or “spatial genomics,” which enables the user to determine simultaneously the expression pattern, or the location/distribution pattern of the genes expressed or genes or genomic loci present in a single cell while retaining information related to the spatial location of the cell within the tissue architecture.

### CROSS-REFERENCE TO RELATED APPLICATIONS

This application claims priority to U.S. Provisional Application No. 62/979,235 filed on Feb. 20, 2020, which is incorporated by reference herein in its entirety.

### FIELD OF THE INVENTION

The present disclosure generally relates to spatial detection of a nucleic acid, such as a genomic DNA or a RNA transcript, in a cell comprised in a tissue sample. The present disclosure provides methods for detecting and/or analyzing nucleic acids, such as chromatin or RNA transcripts, so as to obtain spatial information about the localization, distribution or expression of genes in a tissue sample. The present disclosure thus provides a process for performing “spatial transcriptomics” or “spatial genomics,” which enables the user to determine simultaneously the expression pattern, or the location/distribution pattern of the genes expressed or genes or genomic loci present in a single cell while retaining information related to the spatial location of the cell within the tissue architecture.

### BACKGROUND

Over the past decade, massively-parallel single cell RNA-sequencing (scRNA-seq) has emerged as a powerful approach to catalogue the remarkable cellular heterogeneity in complex tissues (1, 2). While scRNA-seq can profile the transcriptomes of thousands of cells in a single experiment, it requires the dissociation of tissue into single cell suspensions prior to library preparation and sequencing, eliminating any spatial information (3-6). Several strategies have emerged to obtain molecular and spatial information simultaneously from complex tissue. Imaging-based strategy combines high resolution microscopy with fluorescent in situ hybridization (FISH) to achieve subcellular resolution and could profile the entire transcriptome (7-10), but this requires lengthy iterative microscopy workflows and large probe panels. Another approach is to hybridize RNA directly from tissue slices onto a microarray containing spatially-barcoded oligo(dT) spots or beads to encode location information into RNA-sequencing libraries. These approaches can sample the entire transcriptome without the need for iterative rounds of hybridization (11) and recent improvements using DNA-barcoded beads (HDST and Slide-seqvl/v2) report spatial resolutions at or below the diameter of a single cell (12-14). However, because of the low numbers of mRNA molecules captured per bead, these spatial transcriptomic approaches often aggregate neighboring beads prior to downstream analysis, resulting in lower effective resolution and averaging of transcript abundances from multiple cells. As a result, annotation of specific cell types present within each spatial unit of analysis is accomplished by aggregating gene sets computationally defined from orthogonal scRNA-seq datasets (15, 16). While integration methods have demonstrated the ability to localize cell types within the spatial organization of complex tissue, they rely on having available data from two independent assays and have limited ability to infer how spatial context influences the cell state of individual cell types.

### SUMMARY OF THE INVENTION

To address these drawbacks, we have developed XYZeq, a method that expands on recent methods of split-pool indexing (17, 18) for single cell sequencing to enable simultaneous recording of spatial information. At the heart of the approach is a strategy that integrates split-pool indexing and spatial barcoding to enable the profiling, such as transcriptomic profiling or chromatin accessibility profiling, of tens of thousands of single cells and the resolution of cells to thousands of spatial wells. Cellular transcripts, for instance, are spatially encoded in situ with barcoded oligos in an array containing microwells. A tissue slice is placed on an array containing barcoded oligo d(T) primers containing a unique molecular identifier and a PCR handle. This is followed by reverse transcription, split-pool step to introduce a second round of barcoding by PCR, and tagmentation to generate single cell RNA-sequencing libraries. Similar methodology can be used to spatially profile chromatin accessibility. XYZeq compares favorably to both image-based and array- or bead-based methods in its ability to target the genome-wide chromatin or the entire transcriptome and simultaneous estimate single cell gene transcription or expression profiles enabling the detecting of rare and transient transcriptional states.

Accordingly, in one aspect, the present disclosure relates to a method for spatial detection of a nucleic acid within a sample comprising cells, said method comprising identifying presence, absence or quantity of a combination of a spatial barcode domain and a cellular barcode domain in a nucleic acid of the sample.

In some embodiments, the method comprises contacting an array comprising a plurality of microwells with the sample comprising cells such that the sample contacts a plurality of microwells at their distinct positions on the array, wherein each microwell occupies a distinct position on the array and comprises a different spatial index primer comprising a nucleic acid molecule comprising, from 5′ to 3′ :
a) an annealing domain comprising a nucleotide sequence that is recognized by a first sequencing primer;
b) a spatial barcode domain comprising a nucleotide sequence that is unique to each microwell; and
c) a capture domain comprising a polythymidine sequence;.

In some embodiments, the method further comprises allowing a time period to elapse in physiologically acceptable conditions, the time period sufficient to allow hybridization of one or more message RNAs (mRNAs) present in one or more cells located in each microwell to the capture domain of the spatial index primer unique to said microwell. In some embodiments, this step may comprise performing a reverse transcription reaction to obtain a first strand of the cDNA molecules.

In some embodiments, the method further comprises performing reverse transcription to generate one or more cDNA molecules corresponding to the one or more mRNAs present in said microwell. In some embodiments, the method further comprises pooling cells present in each microwell of the array and sorting into a multiwell plate comprising a plurality of wells. In some embodiments, the method further comprises performing an amplification reaction with a cellular index primer comprising a nucleic acid molecule comprising, from 5′ to 3′ :
a) an annealing domain comprising a nucleotide sequence that is recognized by a second sequencing primer; and
b) a cellular barcode domain comprising a nucleotide sequence that is unique to each well of the multiwell plate.

In some embodiments, the method further comprises sequencing amplification reaction products obtained in the above step using the first sequencing primer and the second sequencing primer. In some embodiments, the method further comprises detecting the presence of a nucleotide sequence of a given spatial barcode domain and a nucleotide sequence of a given cellular barcode domain, or sequences complementary to a given spatial barcode domain and a given cellular barcode domain. In some embodiments, the method further comprises a step of providing an array comprising a plurality of microwells prior to contacting each subsample to each spatial index primer. In some embodiments, the method further comprises permeabilizing cells comprised in the tissue sample prior to performing the hybridization. In some embodiments, the method further comprises imaging the array with the sample overlaid after contacting the array with the sample. In some embodiments, the method further comprises lysing the cells after the cells are sorted into the multiwell plate. In some embodiments, the method further comprises generating sequencing libraries from the cDNA molecules generated by tagmentation. In some embodiments, the method further comprises performing an amplification reaction following tagmentation.

In some embodiments, the method further comprises determining which genes are expressed in the cell at a particular distinct location of the tissue sample by a method comprising determining the sequences of the cDNA molecules comprising the same nucleotide sequence of a spatial barcode domain, or sequence complementary thereto, and the same nucleotide sequence of a cellular barcode domain, or sequence complementary thereto. In some embodiments, the method further comprises correlating the nucleotide sequence of a spatial barcode domain unique to a given particular microwell of the array, or the sequence complementary thereto, present in the cDNA molecules to a position in the tissue sample. In some embodiments, the method further comprises correlating the nucleotide sequence of a spatial barcode domain unique to a given particular microwell of the array, or the sequence complementary thereto, present in the cDNA molecules to an image of the tissue sample.

In any of the aforementioned methods, the presence of a particular nucleotide sequence of the spatial barcode domain unique to a given particular microwell of the array, or the sequence complementary thereto, and the presence of a particular nucleotide sequence of the cellular barcode domain, or the sequence complementary thereto, indicates that the cDNA molecules are obtained from mRNAs present in one single cell comprised in the sample at the distinct position where the sample contacted said particular microwell of the assay.

In another aspect, the present disclosure relates to a method of generating a single cell transcriptome profile or RNA library of a sample, the method comprising identifying presence, absence or quantity of a combination of a spatial barcode domain and a cellular barcode domain in a nucleic acid of the sample.

In some embodiments, the method comprises contacting an array comprising a plurality of microwells with the sample comprising cells such that the sample contacts a plurality of microwells at their distinct positions on the array, wherein each microwell occupies a distinct position on the array and comprises a different spatial index primer comprising a nucleic acid molecule comprising, from 5′ to 3′ :
a) an annealing domain comprising a nucleotide sequence that is recognized by a first sequencing primer;
b) a spatial barcode domain comprising a nucleotide sequence that is unique to each microwell; and
c) a capture domain comprising a polythymidine sequence;.

In some embodiments, the method further comprises allowing a time period to elapse in physiologically acceptable conditions, the time period sufficient to allow hybridization of one or more message RNAs (mRNAs) present in one or more cells located in each microwell to the capture domain of the spatial index primer unique to said microwell. In some embodiments, this step may comprise performing a reverse transcription reaction to obtain a first strand of the cDNA molecules.

In some embodiments, the method further comprises performing reverse transcription to generate one or more cDNA molecules corresponding to the one or more mRNAs present in said microwell. In some embodiments, the method further comprises pooling cells present in each microwell of the array and sorting into a multiwell plate comprising a plurality of wells. In some embodiments, the method further comprises performing an amplification reaction with a cellular index primer comprising a nucleic acid molecule comprising, from 5′ to 3′ :
a) an annealing domain comprising a nucleotide sequence that is recognized by a second sequencing primer; and
b) a cellular barcode domain comprising a nucleotide sequence that is unique to each well of the multiwell plate.

In some embodiments, the method further comprises sequencing amplification reaction products obtained in the above step using the first sequencing primer and the second sequencing primer. In some embodiments, the method further comprises detecting the presence of a nucleotide sequence of a given spatial barcode domain and a nucleotide sequence of a given cellular barcode domain, or sequences complementary to a given spatial barcode domain and a given cellular barcode domain. In some embodiments, the method further comprises a step of providing an array comprising a plurality of microwells prior to contacting each subsample to each spatial index primer. In some embodiments, the method further comprises permeabilizing cells comprised in the tissue sample prior to performing the hybridization. In some embodiments, the method further comprises imaging the array with the sample overlaid after contacting the array with the sample. In some embodiments, the method further comprises lysing the cells after the cells are sorted into the multiwell plate. In some embodiments, the method further comprises generating sequencing libraries from the cDNA molecules generated by tagmentation. In some embodiments, the method further comprises performing an amplification reaction following tagmentation.

In some embodiments, the method further comprises determining which genes are expressed in the cell at a particular distinct location of the tissue sample by a method comprising determining the sequences of the cDNA molecules comprising the same nucleotide sequence of a spatial barcode domain, or sequence complementary thereto, and the same nucleotide sequence of a cellular barcode domain, or sequence complementary thereto. In some embodiments, the method further comprises correlating the nucleotide sequence of a spatial barcode domain unique to a given particular microwell of the array, or the sequence complementary thereto, present in the cDNA molecules to a position in the tissue sample. In some embodiments, the method further comprises correlating the nucleotide sequence of a spatial barcode domain unique to a given particular microwell of the array, or the sequence complementary thereto, present in the cDNA molecules to an image of the tissue sample.

The disclosure relates to a method of obtaining the transcriptome of a single cell comprising:
(i) contacting a sample to an array, said array comprising multiple wells comprising
(ii) isolating RNA from the sample in each well;
(iii) performing quantitative PCR on the isolated RNA by amplification of the RNA by the primer or primers in each well;
(iv) correlating the amplification product of the RNA with a cell at a position that corresponds to the position within the sample.
In some embodiments the cell is a mesenchymal cell, a cancer cell, a hepatocyte or a splenocyte.

In any of the aforementioned methods, the presence of a particular nucleotide sequence of the spatial barcode domain unique to a given particular microwell of the array, or the sequence complementary thereto, and the presence of a particular nucleotide sequence of the cellular barcode domain, or the sequence complementary thereto, indicates that the cDNA molecules were obtained from mRNAs present in one single cell comprised in the subsample at the distinct position where the subsample is positioned in said particular microwell of the assay.

In yet another aspect, the present disclosure relates to a method of generating high-resolution spatial positioning of a nucleic acid expression in a cell within a sample, the method comprising identifying presence, absence or quantity of a combination of a spatial barcode domain and a cellular barcode domain in a nucleic acid of the sample.

In some embodiments, the method comprises contacting an array comprising a plurality of microwells with the sample comprising cells such that the sample contacts a plurality of microwells at their distinct positions on the array, wherein each microwell occupies a distinct position on the array and comprises a different spatial index primer comprising a nucleic acid molecule comprising, from 5′ to 3′ :
a) an annealing domain comprising a nucleotide sequence that is recognized by a first sequencing primer;
b) a spatial barcode domain comprising a nucleotide sequence that is unique to each microwell; and
c) a capture domain comprising a polythymidine sequence;.

In some embodiments, the method further comprises allowing a time period to elapse in physiologically acceptable conditions, the time period sufficient to allow hybridization of one or more message RNAs (mRNAs) present in one or more cells located in each microwell to the capture domain of the spatial index primer unique to said microwell. In some embodiments, this step may comprise performing a reverse transcription reaction to obtain a first strand of the cDNA molecules.

In some embodiments, the method further comprises performing reverse transcription to generate one or more cDNA molecules corresponding to the one or more mRNAs present in said microwell. In some embodiments, the method further comprises pooling cells present in each microwell of the array and sorting into a multiwell plate comprising a plurality of wells. In some embodiments, the method further comprises performing an amplification reaction with a cellular index primer comprising a nucleic acid molecule comprising, from 5′ to 3′ :
a) an annealing domain comprising a nucleotide sequence that is recognized by a second sequencing primer; and
b) a cellular barcode domain comprising a nucleotide sequence that is unique to each well of the multiwell plate.

In some embodiments, the method further comprises sequencing amplification reaction products obtained in the above step using the first sequencing primer and the second sequencing primer. In some embodiments, the method further comprises detecting the presence of a nucleotide sequence of a given spatial barcode domain and a nucleotide sequence of a given cellular barcode domain, or sequences complementary to a given spatial barcode domain and a given cellular barcode domain. In some embodiments, the method further comprises a step of providing an array comprising a plurality of microwells prior to contacting each subsample to each spatial index primer. In some embodiments, the method further comprises permeabilizing cells comprised in the tissue sample prior to performing the hybridization. In some embodiments, the method further comprises imaging the array with the sample overlaid after contacting the array with the sample. In some embodiments, the method further comprises lysing the cells after the cells are sorted into the multiwell plate. In some embodiments, the method further comprises generating sequencing libraries from the cDNA molecules generated by tagmentation. In some embodiments, the method further comprises performing an amplification reaction following tagmentation.

In some embodiments, the method further comprises determining which genes are expressed in the cell at a particular distinct location of the tissue sample by a method comprising determining the sequences of the cDNA molecules comprising the same nucleotide sequence of a spatial barcode domain, or sequence complementary thereto, and the same nucleotide sequence of a cellular barcode domain, or sequence complementary thereto. In some embodiments, the method further comprises correlating the nucleotide sequence of a spatial barcode domain unique to a given particular microwell of the array, or the sequence complementary thereto, present in the cDNA molecules to a position in the tissue sample. In some embodiments, the method further comprises correlating the nucleotide sequence of a spatial barcode domain unique to a given particular microwell of the array, or the sequence complementary thereto, present in the cDNA molecules to an image of the tissue sample.

In any of the aforementioned methods, the presence of a particular nucleotide sequence of the spatial barcode domain unique to a given particular microwell of the array, or the sequence complementary thereto, and the presence of a particular nucleotide sequence of the cellular barcode domain, or the sequence complementary thereto, indicates that the cDNA molecule was obtained from the nucleic acid expressed in one single cell comprised in the subsample at the distinct position where the subsample is positioned in said particular microwell of the assay.

In one further aspect, the present disclosure relates to a method of quantifying gene expression in a tissue sample on a single cell level, the method comprising identifying presence, absence or quantity of a combination of a spatial barcode domain and a cellular barcode domain in a nucleic acid of the sample.

In some embodiments, the method comprises contacting an array comprising a plurality of microwells with the sample comprising cells such that the sample contacts a plurality of microwells at their distinct positions on the array, wherein each microwell occupies a distinct position on the array and comprises a different spatial index primer comprising a nucleic acid molecule comprising, from 5′ to 3′ :
a) an annealing domain comprising a nucleotide sequence that is recognized by a first sequencing primer;
b) a spatial barcode domain comprising a nucleotide sequence that is unique to each microwell; and
c) a capture domain comprising a polythymidine sequence;.

In some embodiments, the method further comprises allowing a time period to elapse in physiologically acceptable conditions, the time period sufficient to allow hybridization of one or more message RNAs (mRNAs) present in one or more cells located in each microwell to the capture domain of the spatial index primer unique to said microwell. In some embodiments, this step may comprise performing a reverse transcription reaction to obtain a first strand of the cDNA molecules.

In some embodiments, the method further comprises performing reverse transcription to generate one or more cDNA molecules corresponding to the one or more mRNAs present in said microwell. In some embodiments, the method further comprises pooling cells present in each microwell of the array and sorting into a multiwell plate comprising a plurality of wells. In some embodiments, the method further comprises performing an amplification reaction with a cellular index primer comprising a nucleic acid molecule comprising, from 5′ to 3′ :
a) an annealing domain comprising a nucleotide sequence that is recognized by a second sequencing primer; and
b) a cellular barcode domain comprising a nucleotide sequence that is unique to each well of the multiwell plate.

In some embodiments, the method further comprises sequencing amplification reaction products obtained in the above step using the first sequencing primer and the second sequencing primer. In some embodiments, the method further comprises detecting the presence of a nucleotide sequence of a given spatial barcode domain and a nucleotide sequence of a given cellular barcode domain, or sequences complementary to a given spatial barcode domain and a given cellular barcode domain. In some embodiments, the method further comprises a step of providing an array comprising a plurality of microwells prior to contacting each subsample to each spatial index primer. In some embodiments, the method further comprises permeabilizing cells comprised in the tissue sample prior to performing the hybridization. In some embodiments, the method further comprises imaging the array with the sample overlaid after contacting the array with the sample. In some embodiments, the method further comprises lysing the cells after the cells are sorted into the multiwell plate. In some embodiments, the method further comprises generating sequencing libraries from the cDNA molecules generated by tagmentation. In some embodiments, the method further comprises performing an amplification reaction following tagmentation.

In some embodiments, the method further comprises determining which genes are expressed in the cell at a particular distinct location of the tissue sample by a method comprising determining the sequences of the cDNA molecules comprising the same nucleotide sequence of a spatial barcode domain, or sequence complementary thereto, and the same nucleotide sequence of a cellular barcode domain, or sequence complementary thereto. In some embodiments, the method further comprises correlating the nucleotide sequence of a spatial barcode domain unique to a given particular microwell of the array, or the sequence complementary thereto, present in the cDNA molecules to a position in the tissue sample. In some embodiments, the method further comprises correlating the nucleotide sequence of a spatial barcode domain unique to a given particular microwell of the array, or the sequence complementary thereto, present in the cDNA molecules to an image of the tissue sample.

In any of the aforementioned methods, the presence of a particular nucleotide sequence of the spatial barcode domain unique to a given particular microwell of the array, or the sequence complementary thereto, and the presence of a particular nucleotide sequence of the cellular barcode domain, or the sequence complementary thereto, indicates that the cDNA molecules were obtained from the genes expressed in one single cell comprised in the subsample at the distinct position where the subsample is positioned in said particular microwell of the assay.

In another aspect, the present disclosure relates to a method of spatial detection of a nucleic acid within a sample comprising cells, the method comprising identifying presence, absence or quantity of a combination of a spatial barcode domain and a cellular barcode domain in a nucleic acid of the sample.

In some embodiments, the method further comprises contacting an array comprising a plurality of microwells with the sample comprising cells such that the sample contacts a plurality of microwells at their distinct positions on the array, wherein each microwell occupies a distinct position on the array and comprises an insertional enzyme and a different spatial index adaptor comprising a nucleic acid molecule comprising, from 5′ to 3′:
a) an annealing domain comprising a nucleotide sequence that is recognized by a first sequencing primer; and
b) a spatial barcode domain comprising a nucleotide sequence that is unique to each microwell.

In some embodiments, the method further comprises allowing a time period to elapse in physiologically acceptable conditions, the time period sufficient to allow the insertional enzyme to produce fragments of genomic DNA in one or more cells located in each microwell and tag the fragments of genomic DNA with the spatial index adaptor unique to said microwell.

In some embodiments, the method further comprises pooling cells present in each microwell of the array and sorting into a multiwell plate comprising a plurality of wells.

In some embodiments, the method further comprises performing an amplification reaction with a cellular index primer comprising a nucleic acid molecule comprising, from 5′ to 3′:
a) an annealing domain comprising a nucleotide sequence that is recognized by a second sequencing primer; and
b) a cellular barcode domain comprising a nucleotide sequence that is unique to each well of the multiwell plate.

In some embodiments, the method further comprises sequencing amplification reaction products obtained in step d) using the first sequencing primer and the second sequencing primer.

In some embodiments, the method further comprises detecting the presence of a nucleotide sequence of a given spatial barcode domain and a nucleotide sequence of a given cellular barcode domain, or sequences complementary to a given spatial barcode domain and a given cellular barcode domain. In some embodiments, the method further comprises a step of providing an array comprising a plurality of microwells prior to contacting each subsample to each spatial index primer.

In some embodiments, the insertional enzyme used in any of aforementioned methods is a transposase. In some embodiments, the transposase is Tn5 transposase or MuA transposase.

In any of the aforementioned methods, the presence of a particular nucleotide sequence of the spatial barcode domain unique to a given particular microwell of the array, or the sequence complementary thereto, and the presence of a particular nucleotide sequence of the cellular barcode domain, or the sequence complementary thereto, indicates that the fragments of genomic DNAare obtained from one single cell comprised in the sample at the distinct position where the sample contacted said particular microwell of the assay.

In some embodiments, the one or more cells located in each microwell of the array used in the methods according to the present disclosure are tagged with an antibody. In some embodiments, the methods according to the present disclosure further comprises sorting the one or more cells by the antibody.

In some embodiments, the array used in the methods of the present disclosure comprises at least about 10, 50, 100, 200, 500, 1000, 2000 or 4000 microwells. In some embodiments, the array comprises at least about 768 microwells. In some embodiments, each microwell in the array of the present disclosure is triangle shaped, square shaped, pentagon shaped, hexagon shaped, or round shaped. In some embodiments, each microwell in the array is pentagon shaped.

In some embodiments, each microwell in the array used in the methods of the present disclosure is from about 50 to about 500 microns in depth. In some embodiments, each microwell in the array is about 400 microns in depth.

In some embodiments, the microwells in the array use in the methods of the present disclosure are from about 50 microns to about 500 microns center-to-center space. In some embodiments, the microwells in the array are about 200 microns center-to-center spaced. In some embodiments, the microwells in the array are about 500 microns center-to-center spaced.

In some embodiments, the multiwell plate used in the methods of the present disclosure comprises about 24, 48, 96, 192, 384 or 768 wells. In some embodiments, the multiwell plate comprises about 96 wells. In some embodiments, the multiwell plate comprises about 384 wells. In some embodiments

In some embodiments, about 10 to about 100 cells are sorted into each well of the multiwell plate used in the methods of the present disclosure. In some embodiments, about 20 to about 50 cells are sorted into each well of the multiwell plate.

In some embodiments, the spatial barcode domain comprised in the spatial index primer used in the methods of the present disclosure comprises from about 10 to about 30 nucleotides. In some embodiments, the polythymidine sequence comprised in the spatial index primer used in the methods of the present disclosure comprises from about 10 to about 30 deoxythymidine residues. In some embodiments, the cellular barcode domain comprised in the cellular index primer used in the methods of the present disclosure comprises from about 10 to about 30 nucleotides.

In some embodiments, the sample used in the methods of the present disclosure is a tissue section or a cell suspension. In some embodiments, the sample is a tissue section. In some embodiments, the tissue section is prepared using a fixed tissue, a formalin-fixed paraffin-embedded (FFPE) tissue, or deep-frozen tissue. In some embodiments, the sample is from a subject having, diagnosed with, or suspected of having a tumor.

In another aspect, the present disclosure relates to a system comprising one or a plurality of arrays, each array comprising one or a plurality of microwells, each microwell occupying a distinct position on the array and comprising a spatial index primer comprising a nucleic acid molecule comprising, in 5′ to 3′ orientation:
i) an annealing domain comprising a nucleotide sequence that is recognized by a first sequencing primer;
ii) a spatial barcode domain comprising a nucleotide sequence that is unique to each microwell; and
iii) a capture domain comprising a polythymidine sequence.

In some embodiments, each array of the system according to the present disclosure comprises at least about 10, 50, 100, 200, 500, 1000, 2000 or 4000 microwells. In some embodiments, each array comprises at least about 768 microwells. In some embodiments, each microwell in the array is triangle shaped, square shaped, pentagon shaped, hexagon shaped, or round shaped. In some embodiments, each microwell in the array is pentagon shaped.

In some embodiments, each microwell in the array of the system according to the present disclosure is from about 50 to about 500 microns in depth. In some embodiments, each microwell in the array is about 400 microns in depth. In some embodiments, the microwells in the array are from about 50 microns to about 500 microns center-to-center spaced. In some embodiments, the microwells in the array are about 200 microns center-to-center spaced. In some embodiments, wherein the microwells in the array are about 500 microns center-to-center spaced.

In some embodiments, the system according to the present disclosure further comprises one or a plurality of multiwell plates, each multiwell plate comprising one or a plurality of wells, each well occupying a distinct position on the multiwell plate and comprising a cellular index primer comprising a nucleic acid molecule comprising, from 5′ to 3′:
i) an annealing domain comprising a nucleotide sequence that is recognized by a second sequencing primer; and
ii) a cellular barcode domain comprising a nucleotide sequence that is unique to each well of the multiwell plate.

In some embodiments, the multiwell plate of the system according to the present disclosure comprises about 24, 48, 96, 192, 384 or 768 wells. In some embodiments, the multiwell plate comprises about 96 wells. In some embodiments, the multiwell plate comprises about 384 wells.

In some embodiments, the spatial barcode domain comprised in the spatial index primer used in the array of the system according to the present disclosure comprises from about 10 to about 30 nucleotides. In some embodiments, the polythymidine sequence comprised in the spatial index primer comprises from about 10 to about 30 deoxythymidine residues. In some embodiments, the cellular barcode domain comprised in the cellular index primer comprises from about 10 to about 30 nucleotides.

### BRIEF DESCRIPTION OF THE DRAWINGS

Features of the present disclosure will be understood from the description provided herein, together with the Figures, wherein:

FIG. 1 depicts a general workflow of single cell RNAseq. This platform is typically used to study tissue transcriptomes of homogenized biopsies, which results in averaged transcriptome and loss of spatial information. However, the positional context of gene expression is of key importance to understanding tissue functionality and pathological changes.

FIG. 2 depicts the combinatorial indexing schematic of XYZeq. The combination of spatially informative RT-index and split-pool PCR-index makes it possible to simultaneously obtain transcriptome data at single cell resolution and assign each cell to a specific well in the array. Using two rounds of combinatorial barcoding, for example, first with 768 positional RT-indices and second with 384 PCR-indices, up to 294,912 barcode combinations can be generated.

FIG. 3 depicts the process by which the array for XYZeq is fabricated.

FIGS. 4A-4C depict arrays with hexagonal shaped microwells used for the spatial sequencing platform of the present disclosure. FIG. 4A: Array with 500-micron microwells; FIG. 4B: array with 200-micron microwells; and FIG. 4C: array on a histology slide.

FIGS. 5A-5E illustrate that XYZeq enables single cell and spatial transcriptome profiling simultaneously. FIG. 5A: Schematic of the XYZeq workflow. FIG. 5B: Schematic of XYZeq sequencing library structure. P5 and P7: Illumina adaptors. bp: base pairs. R1 and R2: annealing sites for Illumina sequencing primers. FIG. 5C: Schematic representation of the mixed species cell gradient pattern printed on the chip with 11 unique cell proportion ratios (see Methods in Example 8 for specific cell proportion ratios). FIG. 5D: Scatter plot of mouse (x-axis) and human (y-axis) UMI counts detected from a mixture of HEK293T and NIH3T3 cells after computational decontamination. Dark gray refers to human cells (n=4,182), gray refers to mouse cells (n=2,220), and light gray refers to collisions (n=45). FIG. 5E: Proportion of HEK293T (blue) cells, NIH/3T3 (gray) cells or collisions (light gray) detected by XYZeq for each column of the microwell array.

FIGS. 6A-6C illustrate the high-resolution spatial resolution single cell RNA capture from tissue using XYZeq. FIG. 6A: Scatter plot of transcripts from human (n=XX) and mouse cells (n=XX); FIG. 6B: Violin plot showing the number of detected UMIs and genes per cell; FIG. 6C: Cell distribution spatial map of human and mouse cells in the microarray.

FIGS. 7A-7F show the quantification of specific cell types and gene expression in tissue. FIG. 7A: Annotated cell-identity clusters found by Louvain clustering visualized in a UMAP representation; cell expression to identify hepatocytes (Apoal), tumor (Plec), macrophages (Cd74), liver sinusoidal endothelial cells (Stab2), lymphocytes (Skap1), Kupffer cells (Cd51), from low expression (darker gray) to high expression (light gray). Marker genes may be expressed also in other cell identity populations as shown for macrophages and Kupffer cells; FIG. 7B: Correlation plot comparing XYZeq to 10X chromium; FIG. 7C: Violin plot comparing UMI and gene counts per cell for XYZeq and 10X; FIG. 7D: Heat map representation of cell populations between XYZeq and 10x; FIG. 7E: Spatial density plot showing localization of each cell cluster in the spatial array; FIG. 7F: Spatial pie chart representation that show the ratio of each cell type that occupy each well.

FIGS. 8A-8B show identification of distinct cell populations found in liver tumor model. FIG. 8A: Annotated cell-identity clusters found by Leiden clustering visualized in a UMAP representation; FIG. 8B: Visualization of the overlap of gene expression across the cell populations (the size of the bubble for each gene correlates to the degree of expression for the cell type).

FIG. 9 shows a heat map representing genes that are differentially expressed between celltype clusters with a log-fold change of at least 1.5. The colored bars on the Y axis correspond to the group of genes representative of that cellType cluster.

FIGS. 10A-10G show gene information obtained from the spatial single cell data. The genes tested are a few top marker for lymphocytes and macrophages that showed spatial variation. FIG. 10C, FIG. 10D and FIG. 10G show psuedo time trajectory plots. Each dot represents the macrophage cells. Y axis is the log expression of the gene: in this case TGFbi (FIG. 10C), CCR5 (FIG. 10D) or Tox (FIG. 10G). The horizontal dots on the bottom of FIG. 10C, FIG. 10D and FIG. 10G indicate macrophages that do not express that gene (the macrophages with 0 counts for Tgfbi). The line describes the trend of Tgfb expression across the distance variable. Thus, it is higher at the 0 distance (tumor), then decreases as it moves away (liver). The purple and yellow bar in FIG. 10A and FIG. 10E represents distance, which corresponds to the spatial plot shown in FIG. 10B and FIG. 10F. Yellow is liver, and purple and green are tumor regions. The purple to yellow bar in FIG. 10A and FIG. 10E is the scale/axis for the gene-expression bars above (blue to white). The purple to yellow is a representation the spatial map and the dark blue to white is a representation of the expression of genes in relation to space (specifically tumor to liver).

FIGS. 11A-11D show spatially resolved single cell transcriptomes captured from tissue. FIG. 11A: Scatter plot of mouse (x-axis) and human (y-axis) UMI counts detected from liver/tumor tissues (n=4) at 500 UMI cutoff after decontamination processing. Dark gray on the y-axis refers to human cells (n=2,657) and dark gray on the x-axis refers to mouse cells (n=5,707) and light gray refers to collisions (n=382). FIG. 11B: Violin plots showing the number of detected UMIs (left) and genes (right) per mouse and human cell. Median UMI counts for human cells: 1,596; mouse cells: 1,009. Median gene counts for human cells: 629; mouse cells: 456 across all liver/tumor slices. FIG. 11C: Hematoxylin and eosin (H&E) stained image of the liver/tumor tissue slice. Tumor region (dark gray with light gray dotted outlines); Liver region (light gray). Scale showing 2 mm. FIG. 11D: Visualization of human (gray and dark gray) and mouse (dark gray) cell distribution on the XYZeq array overlayed on the H&E stained slice.

FIGS. 12A-12F show frequency and spatial mapping of single cell clusters from tissue. FIG. 12A: t-distributed stochastic neighbor embedding (tSNE) visualization of the cell types identified from liver/tumor tissue. 6,623 total cells plotted. FIG. 12B: Heat map of scaled marker gene expression and hierarchical clustering of genes that define each cell type from liver/tumor tissue. Reference for grayscale bar in FIG. 12A. FIG. 12C: Correlations of pseudobulk expression values for matching cell types between XYZeq and 10X Genomics Chromium. FIG. 12D: Spatial localization of hepatocytes, MC38 and myeloid cells overlaid on brightfield image of tissue. Light gray dotted outline indicates tumor regions. FIG. 12E: Pie chart of cell type composition for each XYZeq well from a representative liver/tumor tissue slice (top panel) and bar chart illustrating combined cell type composition across all four slices of liver/tumor tissue, which tracks with proximity to the tumor (bottom panel) (see Methods in Example 8 for proximity score). FIG. 12F: Pairplot showing the frequency of hepatocytes, MC38, and myeloid cells in each well. Scatter plots show the colocalization of two cell types in each well. Histograms show the distribution of number of cells (x-axis) per well (y-axis) for each cell type. Pearson correlation (r) and p values are annotated.

FIGS. 13A-13F show expression of gene modules in space that track with cellular composition. FIG. 13A: Projection of average expression of hepatocyte-enriched module (LM14) in tSNE space. Each dot is a cell and colored by the average expression of top contributing module genes (see Methods in Example 8). FIG. 13B: Spatial expression of hepatocyte-enriched module (LM14). Each spatial well is colored by the average expression of top contributing module genes weighted by the number of cells per well. Wells are binarized into high (above weighted average) versus low (all other non-zero expression). Light gray dotted outlines indicate tumor regions. FIG. 13C: Heat map representing the number of overlapping genes between each pair of modules in liver/tumor and spleen/tumor. Each row is a liver module and each column is a spleen module. FIG. 13D: tSNE projection of XYZeq scRNA-seq data grayscaled by annotated cell types in liver/tumor (top left) and spleen/tumor (bottom left) and mean gene expression of the top overlapping modules between liver/tumor (top row) and spleen/tumor (bottom row). Tumor response modules correspond to LM5 and SM12 and immune regulation modules correspond to LM19 and SM7. Projection in spatial coordinates the mean expression of the tumor response modules (FIG. 13E) corresponding to LM5 and SM12; and the immune regulation modules (FIG. 13F) corresponding to LM19 and SM7. Each well in (FIG. 13E, FIG. 13F) are grayscaled by the average gene expression of each module weighted by the number of cells per well (high vs low) and light gray dotted outline indicates tumor regions. Wells are binarized into high (above weighted average) versus low (all other non-zero expression).

FIGS. 14A-14F show differential gene expression within MSCs associated with their spatial proximity to tumor. FIG. 14A: Average expression of the cell migration modules (LM10 and SM17) in tSNE space. Each dot is a cell grayscaled by its mean expression of top module genes between corresponding liver and spleen modules. FIG. 14B: XYZeq array grayscaled by the tumor proximity score. Values near 1 (dark gray) indicate regions rich in tumor, values near 0 (black) indicate regions rich in non-tumor cells, and wells capturing the border between the two tissue types take on values around 0.5 (draker gray). FIG. 14C: MSCs grayscaled by the cell-specific proximity score in tSNE space. FIG. 14D: Row-clustered heat map showing the scaled, mean gene expression in MSCs of genes enriched in three spatial regions (intra-tumor, boundary, intra-tissue) along the 1-dimensional proximity score. For spleen/tumor, statistically significant genes enriched in the tumor and non-tumor regions are highlighted. FIG. 14E: Log expression (y-axis) of Csmd1 (left) and Tshz2 (right) along the proximity score (x-axis). Each dot corresponds to one MSC cell and the regression line is fitted using the negative binomial distribution (see Methods in Example 8). FIG. 14F: Projection in space of mean expression of Csmd1 (left) and Tshz2 (right) in MSCs. Light gray dotted outline indicates tumor region.

FIGS. 15A-15B show that single cell mixed species experiment reveals strong correlation to estimated cell gradient proportions. FIG. 15A: Scatter plot of mouse and human UMI counts detected from a mixture of HEK293T and NIH3T3 cells. Darker gray on the y-axis refers to human cells (n=4,389) and gray on the x-axis refers to mouse cells (n=1,728) and light gray refers to collisions (n=330). FIG. 15B: Scatter plot revealing high concordance between observed and expected cell type proportions in each column of the XYZeq array (Lin’s Concordance Correlation = 0.91).

FIGS. 16A-16C show quantification of cells captured per well from liver/tumor tissue. FIG. 16A: Image of the liver/tumor tissue slice on top of the XYZeq frozen microarray with wells with reagents spotted (white). FIG. 16B: Scatter plot of transcripts (n=4) from human (draker gray on the y-axis: n=2,667), mouse cells (gray on the x-axis: n=6,854), and collisions (light gray: n=747). FIG. 16C: Median cell number in wells across XYZeq array for HEK293T human (top) and liver/tumor mouse (bottom) cells.

FIGS. 17A-17F show distinct cell types clusters identified from XYZeq of liver/tumor tissue. FIG. 17A: tSNE visualization of Leiden cluster to annotated cell types. FIG. 17B: Correlation of mean chromosomal expression of MC38 cells observed in XYZeq compared to MC38 cells from a Efremova, et al. (25), hepatocytes from TabulaMuris (26), and immune cells enriched from liver/tumor from an independent internal experiment (3). Both the x-axis and y-axis denotes average expression of all genes on a given chromosome. FIG. 17C: Violin plot representing estimated contamination fraction for each cell type from our liver/tumor XYZeq data (FIG. 17D, FIG. 17E) Violin plot showing the number of detected UMIs and genes per cell cluster. Median UMI counts (log) and gene counts for each cell cluster: hepatocytes (3.04 and 552), Kupffer cells (2.92 and 420), lymphocytes (2.97 and 454), MSCs (3.08 and 594), macrophages (3.03 and 511), MC38 (3.22 and 851), and LSECs (2.94 and 431). FIG. 17F: Annotated cell-identity clusters; Feature plot of cells that are positive for each individual marker gene to identify Hepatocytes (Cpsl, Glul), MC38 (Plec), macrophages (Cd11b, Cd74), liver sinusoidal endothelial cells (Stab2, Ptprb), lymphocytes (Cd8b, Il18r1), Kupffer cells (Cd5l, Timd4), mesenchymal stem cells (Rbms3, Tshz2), pericentral hepatocytes (Glul, Gluo, Oat) from low expression (black) to high expression (light gray).

FIGS. 18A-18B show reproducibility of XYZeq across tissue slices. Four non-sequential z-layer slices of liver/tumor tissue processed with XYZeq (with HEK293T cells spiked-in as control). FIG. 18A: Pairplot showing the expression of common genes between different slices of liver/tumor. Scatter plots show the UMI counts for common expressed (UMIs > 0) genes. Histograms show the distribution of number of UMIs (x-axis) per gene (y-axis) for each slice. FIG. 18B: tSNE visualization of Leiden clusters across four slices.

FIGS. 19A-19B show that cell type clusters captured from XYZeq found comparable to 10X Genomics platform. FIG. 19A: tSNE representation of liver/tumor tissue data generated with the 10X Chromium V3 kit. 2,703 total cells were plotted. FIG. 19B: Scatter plot comparing the proportion of each cell type found in XYZeq and 10X Chromium V3. Lin’s concordance coefficient of 0.988.

FIGS. 20A-20B show distinct spatial localization pattern across tissue for each cell type cluster. FIG. 20A: Spatial density plot showing localization of lymphocytes, MSCs, Kupffer cells and LSECs in the spatial array. Light gray dotted outline indicates tumor region. FIG. 20B: Pairplot showing the frequency of cell types found in each well across the XYZeq array. Scatter plots show the co-localization of the cell types in each well. Histograms show the distribution for number of cells (x-axis) per well (y-axis) for each cell type. r and p values annotated.

FIGS. 21A-21F show that XYZeq of spleen/tumor tissue reveals comparable data quality to liver/tumor tissue. FIG. 21A: Scatter plot of mouse and human UMI counts detected from spleen/tumor tissues (n=4). Drak gray on the y-axis refers to human cells (n=4,007) and gray on the X-axis refers to mouse cells (n=3,394) and light gray refers to collisions (n=104). FIG. 21B: Violin plot showing the number of detected UMIs and genes per cell. Median UMI counts for human cells: 1,312; mouse cells: 1,169. Median gene counts for human cells: 661; mouse cells: 577. FIG. 21C: H&E stained image of the spleen/tumor tissue slice. Tumor region (gray area with light gray dotted outline); spleen region (darker gray with dark gray features). Scale showing 2 mm. FIG. 21D: Image of spleen/tumor tissue on frozen XYZeq microarray with reagents in wells (white). FIG. 21E: Visualization of human (gray and dark gray) and mouse (gray and dark gray) cell distribution on the XYZeq array with 500 UMI cutoff overlaid on the image of H&E stained tissue slice. FIG. 21F: Median cell number in wells across XYZeq array for HEK293T human (top) and spleen/tumor mouse (bottom) cells.

FIGS. 22A-22D show identification and spatial mapping of cell type clusters from spleen/tumor tissue. FIG. 22A: tSNE projection of the spleen/tumor XYZeq data. 3,394 total cells were plotted. FIG. 22B: tSNE visualization of Leiden cluster to annotate cell types for spleen/tumor. FIG. 22C: Heat map of a scaled expression of marker genes and hierarchical clustering that define each cell type from XYZeq spleen/tumor tissue. FIG. 22D: Image of spleen/tumor tissue overlaid with spatial plot of the XYZeq array showing localization of cell type clusters from (FIG. 22A) with 500 UMI cutoff. Light gray dotted outline indicates tumor region.

FIGS. 23A-23D show cell type contribution and functional annotation of gene modules. FIG. 23A: Barplot showing percent fraction of overlapping genes in liver/tumor modules compared to corresponding spleen/tumor modules. Dotted line represents the threshold used to determine significant overlap between the modules. FIG. 23B: Pie chart representation of cell type fractions that make up each module (see Methods in Example 8). LM denotes liver/tumor module (FIG. 23C, FIG. 23D). GO annotations for tumor response modules (FIG. 23C) and immune regulation modules (FIG. 23D). GO enrichment analysis for immune response module is represented by LM19. p-values computed using GOrilla (50) and adjusted by Benjamini-Hochberg correction.

FIGS. 24A-24B show expression of the cell migration gene module enriched in MSCs. FIG. 24A: Matrix plot of top overlapping genes in the cell migration module (LM10) across all cell types in liver/tumor. FIG. 24B: GO annotation for cell migration module from LM10 and SM17. p-values computed using GOrilla (50) and adjusted by Benjamini-Hochberg correction.

FIGS. 25A-25E show tumor proximity score defined for both liver and spleen tissue. FIG. 25A: The proximity scores for each tissue relied on the annotation of successive concentric layers of neighbors for a well in question. FIG. 25B: The set of wells neighboring each well in the array were tabulated for up to 10 layers. FIG. 25C: The cell-containing wells of representative spleen/tumor slice, where white to lighter gray indicates a higher proportion of tumor cells, and darker gray indicates a higher proportion of non-tumor cells. The wells selected for setting the proximity score to 1 are outlined in white. FIG. 25D: The cell containing wells of a representative liver/tumor slice. Light gray indicates higher proportion of tumor cells, gray to darker gray indicates higher proportion of hepatocytes. FIG. 25E: The proximity score values annotated on each well (left), where lighter gray is closer to the minimum value and darker gray is closer to the maximum value. The scores are visualized for different values of l and d. The values of l = 10 and d = 1.05 were chosen as they rendered the distribution of scores (right) more uniform across all wells.

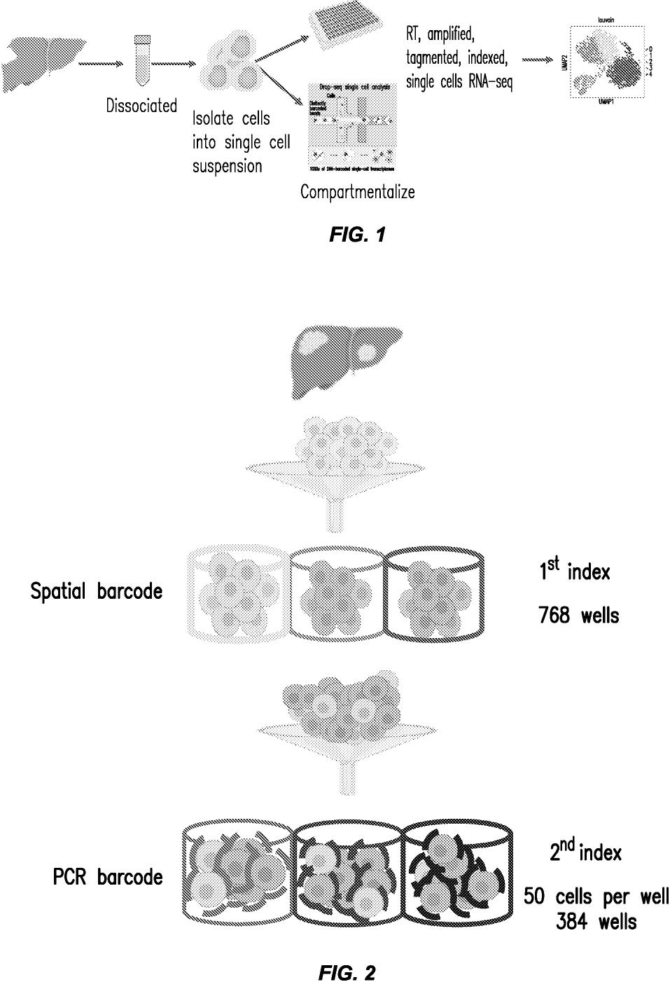

### DETAILED DESCRIPTION OF EMBODIMENTS

The present disclosure can be understood more readily by reference to the following detailed description of embodiments, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

Unless defined otherwise, all technical and scientific terms used herein have the same meaning as commonly understood by one of ordinary skill in the art to which the invention pertains.

As used in the specification and in the claims, the term “comprising” can include the aspects “consisting of” and “consisting essentially of.” Comprising can also mean “including but not limited to.”

As used in the specification and the appended claims, the singular forms “a,” “an” and “the” can include plural referents unless the context clearly dictates otherwise. Thus, for example, reference to “a compound” includes mixtures of compounds; reference to “a pharmaceutical carrier” includes mixtures of two or more such carriers, and the like.

The word “or” as used herein means any one member of a particular list and also includes any combination of members of that list.

The term “about” is used herein to mean within the typical ranges of tolerances in the art. For example, “about” can be understood as about 2 standard deviations from the mean. According to certain embodiments, when referring to a measurable value such as an amount and the like, “about” is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value as such variations are appropriate to perform the disclosed methods. When “about” is present before a series of numbers or a range, it is understood that “about” can modify each of the numbers in the series or range.

As used herein, the term “activated substrate” relates to a material on which interacting or reactive chemical functional groups were oxidated or reduced or otherwise funtionalized by exposure to reagents known to the person skilled in the art to prime the surface for a reaction at the functional group. For example, a substrate comprising carboxyl groups has to be activated before use. Furthermore, there are substrates available that contain functional groups that can react with specific moieties already present in the nucleic acid primers.

As used herein the term “a plurality of” or “multiple” means two or more, or at least two, such as 3, 5, 10, 15, 20, 30, 40, 50, 60, 70, 80, 90, 100, 150, 200, 400, 500, 1000, 2000, 5000, 10,000, or more. Thus, for example, the number of microwells on an array or the number of wells on a multiwell plate may be any integer in any range between any two of the aforementioned numbers.

As used herein, a “cellular index primer” refers to a primer or an oligo for amplifying the cDNA molecules obtained from reverse transcription and labelling each of the amplified cDNA molecules with a second index barcode that is unique to each well of a multiwell plate (defined herein as cellular barcode domains).

As used herein, a “spatial index primer” refers to a primer or an oligo for capturing and labelling transcripts from all of the single cells located at a distinct position in the tissue sample, such as a thin tissue sample slice, or “section.”

An “array,” as that term is used herein, typically refers to an arrangement of entities in spatially discrete locations with respect to one another, and usually in a format that permits simultaneous exposure of the arranged entities to potential interaction partners (e.g., cells) or other reagents, substrates, etc. In some embodiments, an array comprises a solid substrate such as a plastic comprising adjacently arranged microwells in spatially discrete locations on the solid support. In some embodiments, spatially discrete locations on an array are termed “microwells” or “spots” (regardless of their shape). In some embodiments, spatially discrete locations on an array are arranged in a regular pattern with respect to one another (e.g., in a grid). In some embodiments, the array comprise from about 90 to about 400 micrwells arranged in adjacent positions along the planar surface of a solide substrate. In some embodiments, the array is a microarray plate.

The term “barcode” as used herein refers to any unique, non-naturally occurring, nucleic acid sequence capable of identifying the originating source of a nucleic acid fragment. In some embodiments the basrcode is a unique, non-naturally occurring, nucleic acid sequence corresponding to at least one spatial position on an array, such that the barcodes position on the array also corresponds with a position of the cell or cells in contact with that position.

The term “binding” isused broadly throughout this disclosure to refer to any form of attaching or coupling, either non-covalently or covalently, two or more components, entities, or objects. For example, two or more components may be bound to each other via chemical bonds, covalent bonds, ionic bonds, hydrogen bonds, electrostatic forces, Watson-Crick hybridization, etc.In the context of complenmentary nucleic acid seqeunces, two complementary strands bind to form a hydrogen bound duplex of nucleic acid.

The terms “polynucleotide,” “oligo”, “oligonucleotide” and “nucleic acid” are used interchangeably throughout and include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. The nucleic acid molecule can be single-stranded or double-stranded. In some embodiments, the nucleic acid molecules of the disclosure comprise a contiguous open reading frame encoding an antibody, or a fragment thereof, as described herein. “Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may he used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine Nucleic acids may be obtained by chemical synthesis methods or by recombinant methods. A nucleic acid will generally contain phosphodiester bonds, although nucleic acid analogs maybe included that may have at least one different linkage, e.g., phosphoramidate, phosphorothioate, phosphorodithioate, or o-methylphosphoroamidite linkages and peptide nucleic acid backbones and linkages. Other analog nucleic acids include those with positive backbones, non-ionic backbones, and non-ribose backbones, including those described in U.S. Pat. Nos. 5,235,033 and 5,034,506, which are incorporated by reference in their entireties. Nucleic acids containing one or more non-naturally occurring or modified nucleotides are also included within one definition of nucleic acids. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; o- and N-alkylated nucleotides, e.g. N6-methyl adenosine are suitable. The 2′-OH-group may be replaced by a group selected from H, OR, R, halo, SH, SR, NH₂, NHR, N₂ or CN, wherein R is C₁-C₆ alkyl, alkenyl or alkynyl and halo is F, Cl, Br or I. Modified nucleotides also include nucleotides conjugated with cholesterol through, e.g., a hydroxyprolinol linkage as described in Krutzfeldt et al., Nature (Oct. 30, 2005), Soutschek et al., Nature 432:173-178 (2004), and U.S. Pat. Publication No. 20050107325, which are incorporated herein by reference in their entireties. Modified nucleotides and nucleic acids may also include locked nucleic acids (LNA), as described in U.S. Pat. No. 20020115080, which is incorporated herein by reference. Additional modified nucleotides and nucleic acids are described in U.S. Pat. Publication No. 20050182005, which is incorporated herein by reference in its entirety. Modifications of the ribose-phosphate backbone may be done for a variety of reasons, e.g., to increase the stability and half-life of such molecules in physiological environments, to enhance diffusion across cell membranes, or as probes on a biochip. Mixtures of naturally occurring nucleic acids and analogs may be made; alternatively, mixtures of different nucleic acid analogs, and mixtures of naturally occurring nucleic acids and analogs may be made. In some embodiments, the expressible nucleic acid sequence is in the form of DNA. In some embodiments, the expressible nucleic acid is in the form of RNA with a sequence that encodes the polypeptide sequences disclosed herein and, in some embodiments, the expressible nucleic acid sequence is an RNA/DNA hybrid molecule that encodes any one or plurality of polypeptide sequences disclosed herein.

The “percent identity” or “percent homology” of two polynucleotide or two polypeptide sequences is determined by comparing the sequences using the GAP computer program (a part of the GCG Wisconsin Package, version 10.3 (Accelrys, San Diego, Calif.)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may he performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al.). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001. Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

By “substantially identical” is meant nucleic acid molecule (or polypeptide) exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

The term “hybridization” or “hybridizes” as used herein refers to the formation of a duplex between nucleotide sequences that are sufficiently complementary to form duplexes via Watson-Crick base pairing. Two nucleotide sequences are “complementary” to one another when those molecules share base pair organization homology. “Complementary” nucleotide sequences will combine with specificity to form a stable duplex under appropriate hybridization conditions. For instance, two sequences are complementary when a section of a first sequence can bind to a section of a second sequence in an anti-parallel sense wherein the 3′-end of each sequence binds to the 5′-end of the other sequence and each A, T(U), G and C of one sequence is then aligned with a T(U), A, C and G, respectively, of the other sequence. RNA sequences can also include complementary G=U or U=G base pairs. Thus, two sequences need not have perfect homology to be “complementary.” Usually two sequences are sufficiently complementary when at least about 90% (preferably at least about 95%) of the nucleotides share base pair organization over a defined length of the molecule. In the present disclosure, the capture domain of each spatial index primer comprises a region of complementarity for the nucleic acid, e.g. RNA (preferably mRNA) of the tissue sample. In some embodiments, such a region of complementarity comprised in the capture domain of each spatial index primer comprises a polythymidine sequence to capture mRNA via the poly-A tail.

As used herein, the term “sample” refers to a biological sample obtained or derived from a source of interest, as described herein. In some embodiments, a source of interest comprises an organism, such as an animal or human. In some embodiments, a biological sample comprises biological tissue or bodily fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; other body fluids, secretions, and/or excretions; and/or cells therefrom, etc. In some embodiments, a biological sample is or comprises cells obtained from an individual. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, such as organelles, nucleic acid or membrane-bound proteins. In some embodiments, sample is a tissue comprising a plurality of cell types. In some embodiments, sample is connective tissue, muscle tissue, nervous tissue, or epithelial tissue.

The term “amplification reaction” as used herein refers to a reaction by which the number of copies of a nucleic acid is increased. This may be conducted through methods such as polymerase chain reaction (PCR), including but not limited to qPCR, RT-qPCR, RACE-PCR and RT-LAMP, ligase chain reaction (LCR), transcription-mediated amplification, and nicking enzyme amplification reaction (NEAR). Any variation of the aforementioned methodologies for amplifying a nucleic acid is also encompassed by this term.

As used herein, the term “insertional enzyme” refers to an enzyme capable of inserting a nucleic acid sequence into a polynucleotide. In some cases, the insertional enzyme can insert the nucleic acid sequence into the polynucleotide in a substantially sequence-independent manner. The insertional enzyme can be prokaryotic or eukaryotic. Examples of insertional enzymes include, but are not limited to, transposases, HERMES, and HIV integrase. The transposase can be a Tn transposase (e.g., Tn3, Tn5, Tn7, Tn10, Tn552, Tn903), a MuA transposase, a Vibhar transposase (e.g., from Vibrio harveyi), Ac-Ds, Ascot-1, Bs1, Cin4, Copia, En/Spm, F element, hobo, Hsmar1, Hsmar2, IN (HIV), IS1, IS2, IS3, IS4, IS5, IS6, IS10, IS21, IS30, IS50, IS51, IS150, IS256, IS407, IS427, IS630, IS903, IS911, IS982, IS1031, ISL2, L1, Mariner, P element, Tam3, Tc1, Tc3, Te1, THE-1, Tn/O, TnA, Tn3, Tn5, Tn7, Tn10, Tn552, Tn903, Tol1, Tol2, Tn1O, Ty1, any prokaryotic transposase, or any transposase related to and/or derived from those listed above. In certain instances, a transposase related to and/or derived from a parent transposase can comprise a peptide fragment with at least about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% amino acid sequence homology to a corresponding peptide fragment of the parent transposase. The peptide fragment can be at least about 10, about 15, about 20, about 25, about 30, about 35, about 40, about 45, about 50, about 60, about 70, about 80, about 90, about 100, about 150, about 200, about 250, about 300, about 400, or about 500 amino acids in length. For example, a transposase derived from Tn5 can comprise a peptide fragment that is 50 amino acids in length and about 80% homologous to a corresponding fragment in a parent Tn5 transposase. In some cases, the insertion can be facilitated and/or triggered by addition of one or more cations. The cations can be divalent cations such as, for example, Ca²⁺, Mg²⁺ and Mn²⁺.

In some embodiments, the transposase is a DDE motif transposase such as a prokaryotic transposase from ISs, Tn3, Tn5, Tn7, orTn10; a bacteriophage transposase from phage Mu; or a eukaryotic “cut and paste” transposase. U.S. Pat. Nos. 6,593,113; 9,644,199; Yuan and Wessler (2011) Proc Natl Acad Sci USA 108(19):7884-7889. In some embodiments, the transposase includes a retroviral transposase, such asHIV. Rice and Baker (2001) Nat Struct Biol. 8: 302-307.

In some embodiments, the transposase is a member of the IS50 family of transposases, such as Tn5 transposase or variants of Tn5 transposase. Tn5 transposase is derived from the Tn5 transposon, a bacterial transposon that can encode antibiotic resistance genes. The activity of Tn5 transposase can be increased with the point mutations E54K and/or L372P. In particular embodiments, the transposase is a E54K/L372P mutant of Tn5 transposase, which has increased transposase activity. An exemplary E54K/L372P Tn5 transposase comprises the following sequence:

MITSALHRAADWAKSVFSSAALGDPRRTARLVNVAAQLAKYSGKSITISS
EGSKAMQEGAYRFIRNPNVSAEAIRKAGAMQTVKLAQEFPELLAIEDTTS
LSYRHQVAEELGKLGSIQDKSRGWWVHSVLLLEATTFRTVGLLHQEWW
MRPDDPADADEKESGKWLAAAATSRLRMGSMMSNVIAVCDREADIHAY
LQDKLAHNERFVVRSKHPRKDVESGLYLYDHLKNQPELGGYQISIPQKG
VVDKRGKRKNRPARKASLSLRSGRITLKQGNITLNAVLAEEINPPKGETPL
KWLLLTSEPVESLAQALRVIDIYTHRWRIEEFHKAWKTGAGAERQRMEEP
DNLERMVSILSFVAVRLLQLRESFTPPQALRAQGLLKEAEHVESQSAETV
LTPDECQLLGYLDKGKRKRKEKAGSLQWAYMAIARLGGFMDSKRTGIAS
WGALW (SEQ ID NO: 42)

Other mutations to increase the activity of Tn5 transposase are disclosed in U.S. Pat. Nos. 5,965,443; 6,406,896; 7,608,434; and Reznikoff (2003) Molecular Microbiology 47(5): 1199-1206, all of which are expressly incorporated by reference herein. In some embodiments, the Tn5 transposase is a mutant transposase (Tn5-059) with a lowered GC insertion bias. Kia et al. (2017) BMC Biotechnology 17: 6.

### Methods

As mentioned above, methods of the present disclosure relate to a method of the integration of split-pool indexing and spatial barcoding. Thus, the present disclosure uses a set of barcoded index primers to obtained single cell gene expression profiling or transcriptomes from a tissue sample while preserving their corresponding spatial information.

The present disclosure thus relates to a method of spatial recognition of gene expression, the method comprising identifying the presence, absence or quantity of a combination of a spatial barcode domain and a cellular barcode domain in a nucleic acid sample by dtetcing the domain or domains in a sample. In some embodiments, the method further comprises correlating the presence, absence or quantity of the spatial barcode domain and the cellular barcode domain to a spatial position of a cell in a tissue sample on an array.

The present disclosure also relates to a method of identifying a cell type in a sample based on spatial gene expressing profiling, the method comprising detecting the presence, absence or quantity of a combination of a spatial barcode domain and a cellular barcode domain in a sample. In some embodiments, the method further comprises correlating the presence, absence or quantity of the spatial barcode domain and the cellular barcode domain to a spatial position of a cell in a tissue sample on an array. In some embodiments, the step detecting the presence, absence or quantity of a combination of a spatial barcode domain and a cellular barcode domain in a sample comprises annealing one or a plurality of complemtary nucleic acids to the cellular barcode domain and/or the spatial barcode domain and performing a polymerase chain reaction on the sequences to identify the presence or quantity of the one or both domains.

The present disclosure further relates to a method of identifying chromatin accessibility in a cell of a sample, the method comprising identifying the presence, absence or quantity of a combination of a spatial barcode domain and a cellular barcode domain in a nucleic acid sample. In some embodiments, the method further comprises correlating the presence, absence or quantity of the spatial barcode domain and the cellular barcode domain to a spatial position of a cell in a tissue sample on an array.

The present disclosure additionally relates to a method of spatially barcoding a single cell in a tissue, the method comprising identifying or detcting the presence, absence or quantity of a combination of a spatial barcode domain and a cellular barcode domain in a nucleic acid sample. In some embodiments, the method further comprises correlating the presence, absence or quantity of the spatial barcode domain and the cellular barcode domain to a spatial position of a cell in a tissue sample on an array. In some embodiments, the step of detecting comprises detecting a fluorcent signal or probe covalently or non-covalently bound to one or both domains; or detecting one or a plurality of copes of

The present disclosure also relates to a method of spatially identifying a cell population within a tissue, the method comprising identifying the presence, absence or quantity of a combination of a spatial barcode domain and a cellular barcode domain in a nucleic acid sample. In some embodiments, the method further comprises correlating the presence, absence or quantity of the spatial barcode domain and the cellular barcode domain to a spatial position of a cell in a tissue sample on an array.

The present disclosure further relates to a method of detecting gene expression in a single cell in a tissue, the method comprising identifying the presence, absence or quantity of a combination of a spatial barcode domain and a cellular barcode domain in a nucleic acid sample. In some embodiments, the method further comprises correlating the presence, absence or quantity of the spatial barcode domain and the cellular barcode domain to a spatial position of a cell in a tissue sample on an array.

The present disclosure also relates to a method of isolating cells corresponding to a spatial position within a tissue, the method comprising identifying the presence, absence or quantity of a combination of a spatial barcode domain and a cellular barcode domain in a nucleic acid sample. In some embodiments, the method further comprises correlating the presence, absence or quantity of the spatial barcode domain and the cellular barcode domain to a spatial position of the cell in the tissue on an array.

The present disclosure additionally relates to a method of detecting a meschymal stem cell in an organ, the method comprising identifying the presence, absence or quantity of a combination of a spatial barcode domain and a cellular barcode domain in a nucleic acid sample. In some embodiments, the method further comprises correlating the presence, absence or quantity of the spatial barcode domain and the cellular barcode domain to a spatial position of a meschymal stem cell in a tissue sample of the organ on an array.

The present disclosure further relates to a method of quantifying RNA expression in a single cell, the method comprising identifying the presence, absence or quantity of a combination of a spatial barcode domain and a cellular barcode domain in a nucleic acid sample. In some embodiments, the method further comprises correlating the presence, absence or quantity of the spatial barcode domain and the cellular barcode domain to a spatial position of the single cell in a tissue sample on an array.

The present disclosure also relates to a method of quantifying RNA expression corresponding to a spatial position within a tissue sample, the method comprising identifying the presence, absence or quantity of a combination of a spatial barcode domain and a cellular barcode domain in a nucleic acid sample. In some embodiments, the method further comprises correlating the presence, absence or quantity of the spatial barcode domain and the cellular barcode domain to a spatial position of the RNA expression in a tissue sample on an array.

The present disclosure also relates to a method of preparing a nucleic acid of a single cell within a tissue sample, the method comprising identifying the presence, absence or quantity of a combination of a spatial barcode domain and a cellular barcode domain in a nucleic acid sample. In some embodiments, the method further comprises correlating the presence, absence or quantity of the spatial barcode domain and the cellular barcode domain to a spatial position of the nucleci acid sample in the tissue sample on an array.

The disclosure relates to a method of obtaining the transcriptome of a single cell comprising:
(a) contacting a sample to an array, said array comprising multiple wells comprising one or a plurality of spatial primers and/or barcodes;
(b) isolating RNA from the sample in each well;
(c) performing quantitative PCR on the isolated RNA by amplification of the RNA by the annealing th eprimer or primers in each well with the isolated RNA;
(d) correlating the amplification product of the isolated RNA with a cell at a position that corresponds to the position within the sample.
In some embodiments, the cell is a mesenchymal cell, a cancer cell, a hepatocyte or a splenocyte. In some embodiments, the well comprises 1, 2, 3, 4, 5, 6, 7, 8, 9, or 10 cells. In some embodiments, the method further comprises repeating the steps over each well to create an expression profile; and calculating an average of mean expression across an expression profile for each well weighted by the number of cells in each well.

In some embodiments, the methods further comprise a step of calculating a proximity score. In some embodiments, the step of calculating the proximity score comprises performing the analysis on page 88 of the specification. In some embodiments, the methods further comprise perfroming a trajectory interference anaylsis.

The disclosure relates to a method of obtaining the transcriptome of a single cell comprising:
(a) contacting a sample to an array, said array comprising multiple wells comprising
(b) isolating RNA from the sample in each well;
(c) performing quantitative PCR on the isolated RNA by amplification of the RNA by the primer or primers in each well;
(d) correlating the amplification product of the RNA with a cell at a position that corresponds to the position within the sample;
wherein each well comprises barcode and a primer that correspond to the position of the barcode and the primer within the array.

The term “barcode” as used herein refers to any unique, non-naturally occurring, nucleic acid sequence capable of identifying the originating source of a nucleic acid fragment. The barcode sequence provides a high-quality individual read of a barcode associated with, for instance, DNA, RNA, cDNA, cell or nuclei, such that multiple species can be sequenced together.

Barcoding may be performed based on any of the compositions or methods disclosed in Pat. Publication WO 2014/047561 A1, which is incorporated herein by reference in its entirety. Not being bound by a theory, amplified sequences from single cells or nuclei can be sequenced together and resolved based on the barcode associated with each cell or nuclei. Other barcoding designs and tools have also been described (see e.g., Birrell et al., (2001) Proc. Natl. Acad. Sci. USA 98:12608-12613; Giaever, et al., (2002) Nature 418: 387-391; Winzeler et al., (1999) Science 285:901-906; and Xu et al., (2009) Proc. Natl. Acad. Sci. USA. 106:2289-2294).

A first barcoded index primer of the present disclosure is called “spatial index primer.” As used herein, a “spatial index primer” refers to a primer or an oligo for capturing and labelling transcripts from all of the single cells located at a distinct position in the tissue sample, such as a thin tissue sample slice, or “section.” The tissue samples or sections for analysis are produced in a highly parallelized fashion, such that the spatial information in the section is preserved. The captured RNA molecules, preferably mRNAs, for each cell, or “transcriptomes,” are subsequently transcribed into cDNA molecules and the resultant cDNA molecules are analyzed, for example, by high throughput sequencing. The resultant data may be correlated to images of the original tissue samples, such as sections, through the barcode sequences (or ID tags, defined herein as spatial barcode domains) incorporated into the arrayed nucleic acids via the spatial index primers.

To accomplish all of these functions, each “spatial index primer,” according to the present disclosure, comprises at least two domains, a capture domain and a spatial barcode domain (or spatial tag). The spatial index primer may further comprise a universal domain as defined further below.

In some embodiments, the capture domain is located at the 3′ end of the spatial index primer and comprises a free 3′ end that can be extended by, for example, template dependent polymerization. The capture domain comprises a nucleotide sequence that is capable of hybridizing to a nucleic acid, e.g. RNA (preferably mRNA), present in the cells of the tissue sample contact with the array. In some embodiments where transcriptional profiling is preferred, the capture domain may comprise a polythymidine sequence, such as a poly-T (or a “poly-T-like”) oligonucleotide, alone or in conjunction with a random oligonucleotide sequence. The random oligonucleotide sequence, if used, may for example be located 5′ or 3′ of the poly-T sequence, such as at the 3′ end of the spatial index primer.

In some embodiments, the spatial barcode domain (or spatial tag) of the spatial index primer comprises a nucleotide sequence which is unique to each microwell of an array and acts as a positional or spatial marker (the identification tag). In this way, each region or domain of the tissue sample, e.g. each cell in the tissue, will be identifiable by spatial resolution across the array linking the nucleic acid, such as RNAs or transcripts, from a certain cell to a unique spatial barcode domain sequence in the spatial index primer. By virtue of the spatial barcode domain, a spatial index primer in the array may be correlated to a position in the tissue sample, for instance, it may be correlated to a cell in the tissue sample. In some embodiments, the spatial resolution at a particular position is from about 0.1 µm² to about 1 cm². In some embodiments, the spatial resolution at a particular position is about 0.1 µm². In some embodiments, the spatial resolution at a particular position is about 0.2 µm². In some embodiments, the spatial resolution at a particular position is about 0.5 µm². In some embodiments, the spatial resolution at a particular position is about 0.75 µm². In some embodiments, the spatial resolution at a particular position is about 1 µm². In some embodiments, the spatial resolution at a particular position is about 2 µm². In some embodiments, the spatial resolution at a particular position is about 5 µm². In some embodiments, the spatial resolution at a particular position is about 10 µm². In some embodiments, the spatial resolution at a particular position is about 20 µm². In some embodiments, the spatial resolution at a particular position is about 30 µm². In some embodiments, the spatial resolution at a particular position is about 50 µm². In some embodiments, the spatial resolution at a particular position is about 80 µm². In some embodiments, the spatial resolution at a particular position is about 100 µm². In some embodiments, the spatial resolution at a particular position is about 150 µm². In some embodiments, the spatial resolution at a particular position is about 200 µm². In some embodiments, the spatial resolution at a particular position is about 500 µm². In some embodiments, the spatial resolution at a particular position is about 750 µm². In some embodiments, the spatial resolution at a particular position is about 1 cm².

Any suitable sequence may be used as the spatial barcode domain in the spatial index primer according to the present disclosure. By a suitable sequence, it is meant that the spatial barcode domain does not interfere with (i.e. inhibit or distort) the interaction between the RNA of the tissue sample and the capture domain of the spatial index primer. For example, the spatial barcode domain should be designed such that nucleic acid molecules in the tissue sample do not hybridize specifically or substantially to the spatial barcode domain or a complementary portion thereof. In some embodiments, the nucleotide sequence of the spatial barcode domain of the spatial index primer, or the complementary thereof, has less than about 80% sequence identity across a substantial part of the nucleic acid molecules in the tissue sample. In some embodiments, the nucleotide sequence of the spatial barcode domain of the spatial index primer, or the complementary thereof, has less than about 70% sequence identity across a substantial part of the nucleic acid molecules in the tissue sample. In some embodiments, the nucleotide sequence of the spatial barcode domain of the spatial index primer, or the complementary thereof, has less than about 60% sequence identity across a substantial part of the nucleic acid molecules in the tissue sample. In some embodiments, the nucleotide sequence of the spatial barcode domain of the spatial index primer, or the complementary thereof, has less than about 50% sequence identity across a substantial part of the nucleic acid molecules in the tissue sample. In some embodiments, the nucleotide sequence of the spatial barcode domain of the spatial index primer, or the complementary thereof, has less than about 40% sequence identity across a substantial part of the nucleic acid molecules in the tissue sample. Sequence identity may be determined by any appropriate method known in the art, such as using the BLAST alignment algorithm.

The nucleotide sequence of the spatial barcode domain of the spatial index primer may be generated using random sequence generation. The randomly generated sequences may be followed by stringent filtering by mapping to the genomes of all common reference species and with pre-set Tm intervals, GC content and a defined distance of difference to the other barcode sequences to ensure that the barcode sequences will not interfere with the capture of the nucleic acid, e.g. RNA from the tissue sample, and will be distinguishable from each other without difficulty.

As mentioned above, in some embodiments, the spatial index primer further comprises a universal domain. In some embodiments, the universal domain of the spatial index primer is located directly or indirectly upstream, i.e. closer to the 5′ end of the spatial index primer, of the spatial barcode domain. In some embodiments, the universal domain is directly adjacent to the spatial barcode domain, i.e. there is no intermediate sequence between the spatial barcode domain and the universal domain. In embodiments where the spatial index primer comprises a universal domain, the domain can form the 5′ end of the spatial index primer, which may be immobilized directly or indirectly on the substrate of the array.

As described elsewhere herein, the cDNA molecules obtained from the RNA molecules, preferably mRNAs, captured by the capture domains of the spatial index primers are subsequently sequenced and analyzed. Thus, in some embodiments, the universal domain comprised in the spatial index primer may comprise an annealing domain comprising a nucleotide sequence that is recognized by a first sequencing primer. To sequence and analyze the cDNA molecules in a high-throughput manner, in some embodiments, the annealing domain in each spatial index primer preferably comprises the same nucleotide sequence.

Any suitable sequence may be used as the annealing domain in the spatial index primers of the present disclosure. By a suitable sequence, it is meant that the annealing domain should not interfere with (i.e. inhibit or distort) the interaction between the nucleic acid, e.g. RNA of the tissue sample, and the capture domain of the spatial index primer. Furthermore, the annealing domain should comprise a nucleotide sequence that is not the same or substantially the same as any sequence in the nucleic acid, e.g. RNA of the tissue sample, such that the primer used for the sequencing can hybridized only to the annealing domain under the conditions used for the sequencing.

For example, the annealing domain should be designed such that nucleic acid molecules in the tissue sample do not hybridize specifically to the annealing domain or the complementary thereof. In some embodiments, the nucleotide sequence of the annealing domain of the spatial index primer, or the complementary thereof, has less than about 80% sequence identity across a substantial part of the nucleic acid molecules in the tissue sample. In some embodiments, the nucleotide sequence of the annealing domain of the spatial index primer, or the complementary thereof, has less than about 70% sequence identity across a substantial part of the nucleic acid molecules in the tissue sample. In some embodiments, the nucleotide sequence of the annealing domain of the spatial index primer, or the complementary thereof, has less than about 60% sequence identity across a substantial part of the nucleic acid molecules in the tissue sample. In some embodiments, the nucleotide sequence of the annealing domain of the spatial index primer, or the complementary thereof, has less than about 50% sequence identity across a substantial part of the nucleic acid molecules in the tissue sample. In some embodiments, the nucleotide sequence of the annealing domain of the spatial index primer, or the complementary thereof, has less than about 40% sequence identity across a substantial part of the nucleic acid molecules in the tissue sample. Sequence identity may be determined by any appropriate method known in the art, such as using the BLAST alignment algorithm.

The second barcoded index primer of the present disclosure is called “cellular index primer.” As used herein, a “cellular index primer” refers to a primer or an oligo for amplifying the cDNA molecules obtained from reverse transcription and labelling each of the amplified cDNA molecules with a second index barcode that is unique to each well of a multiwell plate (defined herein as cellular barcode domains). As described elsewhere herein, this step of PCR amplification to amplified the cDNA molecules obtained from reverse transcription is performed on a multiwell plate instead of the array on which the first barcoded index primer of the present disclosure is incorporated into arrayed nucleic acids via the spatial index primers.

According to the present disclosure, each “cellular index primer” comprises at least one domain called “cellular barcode domain” (or cellular tag). The cellular index primer may further comprise a universal domain as defined further below.

The cellular barcode domain (or cellular tag) of the cellular index primer comprises a nucleotide sequence which is unique to each well of the multiwell plate and acts as an identification tag for the cells located in any given well of the multiwell plate. In this way, all the PCR products obtained from the PCR amplification in each well are labelled with the same cellular barcode domain. Transcripts of a single cell at a particular location on the array can thus be identified based on the combination of a specific spatial barcode domain and a specific cellular barcode domain. The disclosure relates to a method of spatial recognition of gene expression comprising identifying a spatial barcode domain and a specific cellular barcode domain.

Any suitable sequence may be used as the cellular barcode domain in the cellular index primer according to the present disclosure. By a suitable sequence, it is meant that, for example, the cellular barcode domain is designed such that cDNA molecules obtained from reverse transcription do not hybridize specifically or substantially to the cellular barcode domain or a complementary thereof. In some embodiments, the nucleotide sequence of the cellular barcode domain of the cellular index primer, or the complementary thereof, has less than about 80% sequence identity across a substantial part of the cDNA molecules obtained from reverse transcription. In some embodiments, the nucleotide sequence of the cellular barcode domain of the cellular index primer, or the complementary thereof, has less than about 70% sequence identity across a substantial part of the cDNA molecules obtained from reverse transcription. In some embodiments, the nucleotide sequence of the cellular barcode domain of the cellular index primer, or the complementary thereof, has less than about 60% sequence identity across a substantial part of the cDNA molecules obtained from reverse transcription. In some embodiments, the nucleotide sequence of the cellular barcode domain of the cellular index primer, or the complementary thereof, has less than about 50% sequence identity across a substantial part of the cDNA molecules obtained from reverse transcription. In some embodiments, the nucleotide sequence of the cellular barcode domain of the cellular index primer, or the complementary thereof, has less than about 40% sequence identity across a substantial part of the cDNA molecules obtained from reverse transcription. Sequence identity may be determined by any appropriate method known in the art, such as using the BLAST alignment algorithm.

The nucleotide sequence of the cellular barcode domain of the cellular index primer may be generated using random sequence generation. The randomly generated sequences may be followed by stringent filtering by mapping to the genomes of all common reference species and with pre-set Tm intervals, GC content and a defined distance of difference to the other barcode sequences to ensure that the barcode sequences will not hybridize to the cDNA molecules obtained from reverse transcription and will be distinguishable from each other without difficulty.

As mentioned above, the cellular index primer may also comprise a universal domain. The universal domain of the cellular index primer is located directly or indirectly upstream, i.e. closer to the 5′ end of the cellular index primer, of the cellular barcode domain. In some embodiments, the universal domain is directly adjacent to the cellular barcode domain, i.e. there is no intermediate sequence between the cellular barcode domain and the universal domain. In embodiments where the cellular index primer comprises a universal domain, the domain will form the 5′ end of the cellular index primer, which may be immobilized directly or indirectly on the substrate of the multiwell plate.

As described elsewhere herein, the cDNA molecules obtained from reverse transcription followed by PCR amplification are subsequently sequenced and analyzed. Thus, in some embodiments, the universal domain comprised in the cellular index primer may comprise an annealing domain comprising a nucleotide sequence that is recognized by or complentary to a second sequencing primer. To sequence and analyze the cDNA molecules in a high-throughput manner, in some embodiments, the annealing domain in each cellular index primer preferably comprises the same nucleotide sequence.

Any suitable sequence may be used as the annealing domain in the cellular index primers of the present disclosure. By a suitable sequence, it is meant that, for example, the annealing domain of any given cellular index primer should comprise a nucleotide sequence that is not the same or not substantially the same as any sequence in the cDNA molecules obtained from reverse transcription such that the primer used for the sequencing can hybridized only to the annealing domain under the conditions used for the sequencing.

For example, the annealing domain should be designed such that nucleic acid molecules in the tissue sample do not hybridize specifically to the annealing domain or the complementary sequence thereof. In some embodiments, the nucleotide sequence of the annealing domain of the cellular index primer, or the complementary thereof, has less than about 90%, 85%, 80%. 75% or 70% sequence identity across a substantial part of the nucleic acid molecules in the tissue sample. In some embodiments, the nucleotide sequence of the annealing domain of the cellular index primer, or the complementary sequence thereof, has less than about 70% sequence identity across a substantial part of the nucleic acid molecules in the tissue sample. In some embodiments, the nucleotide sequence of the annealing domain of the cellular index primer, or the complementary thereof, has less than about 60% sequence identity across a substantial part of the nucleic acid molecules in the tissue sample. In some embodiments, the nucleotide sequence of the annealing domain of the cellular index primer, or the complementary thereof, has less than about 50% sequence identity across a substantial part of the nucleic acid molecules in the tissue sample. In some embodiments, the nucleotide sequence of the annealing domain of the cellular index primer, or the complementary thereof, has less than about 40% sequence identity across a substantial part of the nucleic acid molecules in the tissue sample. Sequence identity may be determined by any appropriate method known in the art, such as using the BLAST alignment algorithm.

The array, or microwell array, according to the present disclosure may contain multiple or a plurality of microwells. A microwell may be defined by a volume, area or distinct position on the array. In some embodiments, a single species of spatial index primer is immobilized or in solution. In some embodiments, the disclosure relates to a system comprising an array, wherein the array comprises 6, 12, 24, 48, 96, 192 or more microwells. In some embodiments, each microwell will comprise a multiplicity of spatial index primer molecules of the same species. It will be understood in this context that, while it is encompassed that each spatial index primer of the same species may have the same sequence, this need not necessarily be the case. In some embodiments, each species of spatial index primer will have the same spatial barcode domain (i.e. each member of a species and thus each primer in a microwell will be identically “tagged”), but the sequence of each member of the microwell (species) may differ, because the sequence of a capture domain may differ. As described above, random nucleic acid sequences may be included in the capture domains.

In some embodiments, the spatial index primers within a microwell may comprise different random sequences. The number and density of the microwells on the array will determine the resolution of the array, i.e. the level of detail at which the transcriptome of the tissue sample can be analyzed. A higher density of microwells will typically increase the resolution of the array. As mentioned above, the methods of the present disclosure provide a spatial recognition of gene expression based on a specific combination of a spatial barcode domain and a cellular barcode domain, the present disclosure provides a resolution at a single cell level. However, the tissue resolution will depend on the size of microwells. Accordingly, in some embodiments, the array comprises a plurality of microwells, each microwell equidistant from each other and comprising a volume of from about 100 to 400 microliters. In some embodiments, the array comprises a plurality of microwells, each microwell equidistant from each other (as measured by the center of each well) and comprise a volume of from about 100 to 400 microliters. In some embodiments, the array comprises a plurality of microwells, each microwell equidistant from each other (as measured by the center of each well) and comprise a volume of from about 10 to 400 microliters. In some embodiments, the array comprises a plurality of microwells, each microwell equidistant from each other (as measured by the center of each well) and comprise a volume of from about 20 to about 400 microliters. In some embodiments, the array comprises a plurality of microwells, each microwell equidistant from each other (as measured by the center of each well) and comprise a volume of from about 50 to about 400 microliters. In some embodiments, the array comprises a plurality of microwells, each microwell equidistant from each other (as measured by the center of each well) and comprise a volume of from about 75 to about 350 microliters. In some embodiments, the array comprises a plurality of microwells, each microwell equidistant from each other (as measured by the center of each well) and comprise a volume of from about 100 to 370 microliters. In some embodiments, the array comprises a plurality of microwells, each microwell equidistant from each other (as measured by the center of each well) and comprise a volume of from about 300 to about 375 microliters. In some embodiments, the array comprises a plurality of microwells, each microwell equidistant from each other (as measured by the center of each well) and comprise a volume of from about 340 to about 360 microliters. In some embodiments, the array comprises a plurality of microwells, each microwell equidistant from each other (as measured by the center of each well) and comprise a volume of from about 5 to about 100 microliters. In some embodiments, the array comprises a plurality of microwells, each microwell equidistant from each other (as measured by the center of each well) and comprises a barcode index primer immobilized on the bottom of each microwell of the array.

In some embodiments, the methods are capable of detecting and expression profle with a spatial resolution at a particular position of a sample from about 0.1 µm² to about 1 cm² of the sample. In some embodiments, the spatial resolution at a particular position of the sample is about 0.1 µm². In some embodiments, the spatial resolution at a particular position of the sample is about 0.2 µm². In some embodiments, the spatial resolution at a particular position of the sample is about 0.5 µm². In some embodiments, the spatial resolution at a particular position of the sample is about 0.75 µm². In some embodiments, the spatial resolution at a particular position of the sample is about 1 µm². In some embodiments, the spatial resolution at a particular position of the sample is about 2 µm². In some embodiments, the spatial resolution at a particular position of the sample is about 5 µm². In some embodiments, the spatial resolution at a particular position of the sample is about 10 µm². In some embodiments, the spatial resolution at a particular position of the sample is about 20 µm². In some embodiments, the spatial resolution at a particular position of the sample is about 30 µm². In some embodiments, the spatial resolution at a particular position of the sample is about 50 µm². In some embodiments, the spatial resolution at a particular position of the sample is about 80 µm². In some embodiments, the spatial resolution at a particular position of the sample is about 100 µm². In some embodiments, the spatial resolution at a particular position of the sample is about 150 µm². In some embodiments, the spatial resolution at a particular position of the sample is about 200 µm². In some embodiments, the spatial resolution at a particular position of the sample is about 500 µm². In some embodiments, the spatial resolution at a particular position of the sample is about 750 µm². In some embodiments, the spatial resolution at a particular position of the sample is about 1 cm².

As mentioned above, the size and number of the microwells on the array of the present disclosure will depend on the nature of the sample and required resolution. For example, if the sample contains large cells, then the number and/or density of microwells on the array may be reduced (i.e. lower than the possible maximum number of microwells) and/or the size of the microwells may be increased (i.e. the area of each microwell may be greater than the smallest possible microwell), such as an array comprising few large microwells. Alternatively, if it is desirable to increase the resolution or the tissue sample contains small cells, it may be necessary to use the maximum number of microwells possible, which would necessitate using the smallest possible microwell size, such as an array comprising many small microwells.

Accordingly, in some embodiments, an array of the present disclosure may contain at least about 2, about 5, about 10, about 50, about 100, about 500, about 750, about 1000, about 1500, about 2000, about 2500, about 3000, about 3500, about 4000, about 4500 or about 5000 microwells. In other embodiments, arrays with microwells in excess of about 5000 may be prepared and such arrays are envisaged and within the scope of the present disclosure. As noted above, microwell size may be decreased and this may allow greater numbers of microwells to be accommodated within the same or a similar area. By way of example, these microwells may be comprised in an area of less than about 20 cm², about 10 cm², about 5 cm², about 1 cm², about 1 mm², or about 100 µm².

Depending on the size of the microwells and the area in which they are comprised, the microwells of the present disclosure may be from about 50 microns to about 500 microns center-to-center spaced. In some embodiments, the microwells are about 50 microns center-to-center spaced. In some embodiments, the microwells are about 100 microns center-to-center spaced. In some embodiments, the microwells are about 150 microns center-to-center spaced. In some embodiments, the microwells are about 200 microns center-to-center spaced. In some embodiments, the microwells are about 250 microns center-to-center spaced. In some embodiments, the microwells are about 300 microns center-to-center spaced. In some embodiments, the microwells are about 350 microns center-to-center spaced. In some embodiments, the microwells are about 400 microns center-to-center spaced. In some embodiments, the microwells are about 450 microns center-to-center spaced. In some embodiments, the microwells are about 500 microns center-to-center spaced.

The microwells of the present disclosure may be in any desired shape, including but not limited to stacked planar triangles, squares, pentagons, hexagons, or are cylindrical. In some embodiments, the microwells are triangle shaped. In some embodiments, the microwells are square shaped. In some embodiments, horizontal planes of the microwells are pentagon-shaped. In some embodiments, the microwells are hexagonal. In some embodiments, the microwells ar cylindrical with round bottomsat the base.

As illustrated in the accompanied drawings, in some embodiments, the microwells according to the present disclosure have a 3-dimensional structure rather than a 2-dimensional, flat surface. In some embodiments, the microwells of the present disclosure have a depth of about 5 µm, about 10 µm, about 50 µm, about 100 µm, about 150 µm, about 200 µm, about 250 µm, about 300 µm, about 350 µm, about 400 µm, about 450 µm, or about 500 µm. In other embodiments, depending on the application and the tissue sample, arrays with microwells having a depth of more than about 500 µm may be prepared and such arrays are envisaged and within the scope of the present disclosure. In some embodiments, the depth is from about 1 µm to about 1000 µm.

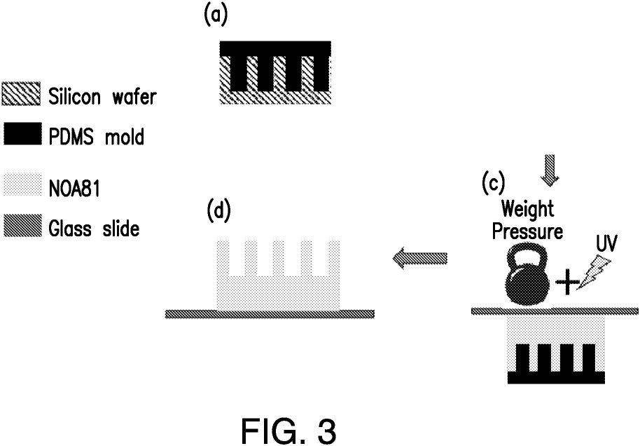

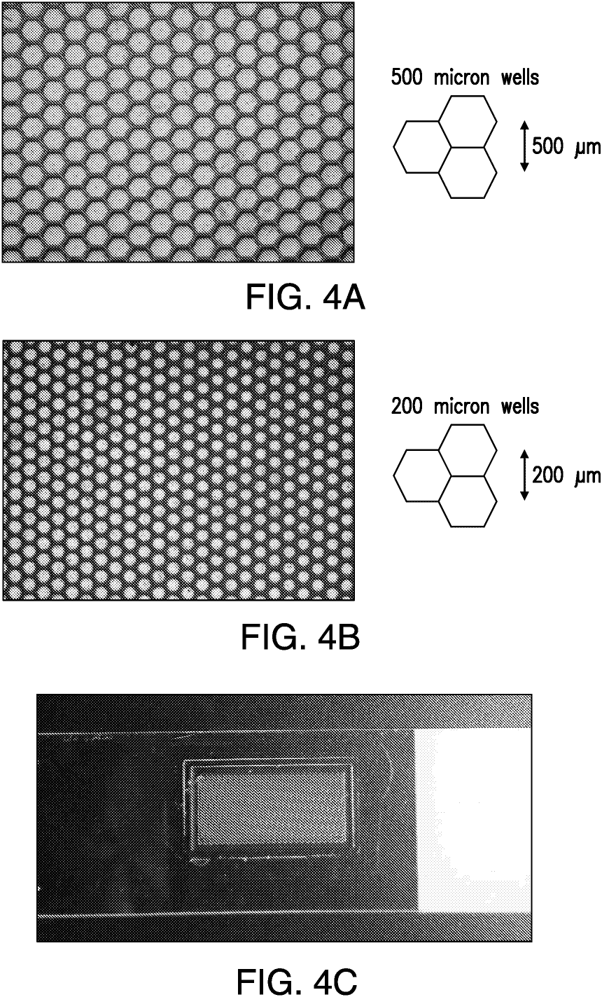

The array, or microwell array, according to the present disclosure may be fabricated using any suitable material known to the person skilled in the art. Typically, a positive mold and a negative mold will be needed to fabricate the microwell array. In some embodiments, a negative mold, which is the reverse template of the microwells, can be fabricated using, for example, silicon wafer with microwells. Microwells with desired size, shape and spacing are then fabricated on a solid support, such as glass, plastic or silicon chip or slide, using the resultant negative mold. A non-limiting example of microwell array fabrication is provided in the examples below and illustrated in FIG. 3.

The multiwell plate according to the present disclosure, by definition, contains multiple or a plurality of wells. In some embodiments, the multiwell plate of the present disclosure contains about 4, about 16, about 32, about 48, about 96, about 192, about 384, about 768 or about 1536 wells. In other embodiments, multiwell plate with wells in excess of about 1536 may be used and such multiwell plates are envisaged and within the scope of the present disclosure. In some embodiments, the multiwell plate of the present disclosure is a microplate or microtiter plate.

Similar to the microwell described above, each well of the multiwell plate may be defined as an area or distinct position on the microwell plate at which a single species of cellular index primer is immobilized. Thus, each well will comprise a multiplicity of cellular index primer molecules of the same species. It will be understood in this context that, whilst it is encompassed that each cellular index primer of the same species may have the same sequence, this need not necessarily be the case. Each species of cellular index primer will have the same cellular barcode domain (i.e. each member of a species and thus each primer in a well will be identically “tagged”), but the sequence of each member of the well (species) may differ. As described above, the cellular index primer may comprise a universal domain, which can be directly or indirectly adjacent to the cellular barcode domain. Thus, the cellular index primers within a particular well may comprise different intermediate sequence in between the cellular barcode domain and the universal domain.

The spatial index primers and cellular index primers may be attached to the microwells of the array or the wells of the multiwell plate, respectively, by any suitable means. In some embodiments, the spatial index primers and cellular index primers are immobilized to the microwells or wells by chemical immobilization. This may be an interaction between the substrate (support material) of the array or plate and the spatial index primer or cellular index primer based on a chemical reaction. Such a chemical reaction typically does not rely on the input of energy via heat or light, but can be enhanced by either applying heat, e.g. a certain optimal temperature for a chemical reaction, or light of certain wavelength. For example, a chemical immobilization may take place between functional groups on the substrate and corresponding functional elements on the spatial index primer or cellular index primer. Such corresponding functional elements in the spatial index primer or cellular index primer may either be an inherent chemical group of the primer, e.g. a hydroxyl group or be additionally introduced. An example of such a functional group is an amine group. Typically, the spatial index primer or cellular index primer to be immobilized comprises a functional amine group or is chemically modified in order to comprise a functional amine group. Means and methods for such a chemical modification are well known.

The localization of such a functional group within the spatial index primer or cellular index primer to be immobilized may be used in order to control and shape the binding behavior and/or orientation of the primer, e.g. the functional group may be placed at the 5′ or 3′ end of the spatial index primer or cellular index primer or within sequence of the primer. A typical substrate for a spatial index primer or cellular index primer to be immobilized comprises moieties which are capable of binding to such primers, e.g. to amine-functionalized nucleic acids. Examples of such substrates are carboxy, aldehyde or epoxy substrates. Such materials are known to the person skilled in the art. Functional groups, which impart a connecting reaction between primers which are chemically reactive by the introduction of an amine group, and array substrates are known to the person skilled in the art.

Alternative substrates on which spatial index primers or cellular index primers may be immobilized may have to be chemically activated, e.g. by the activation of functional groups, available on the array substrate or plate substrate. The term “activated substrate” relates to a material in which interacting or reactive chemical functional groups were established or enabled by chemical modification procedures as known to the person skilled in the art. For example, a substrate comprising carboxyl groups has to be activated before use. Furthermore, there are substrates available that contain functional groups that can react with specific moieties already present in the nucleic acid primers.

Typically, the substrate is a solid support and thereby allows for an accurate and traceable positioning of the nucleic acid primers on the substrate. An example of a substrate is a solid material or a substrate comprising functional chemical groups, e.g. amine groups or amine-functionalized groups. A substrate envisaged by the present disclosure is a non-porous substrate. Preferred non-porous substrates are glass, silicon, poly-L-lysine coated material, nitrocellulose, polystyrene, cyclic olefin copolymers (COCs), cyclic olefin polymers (COPs), polypropylene, polyethylene and polycarbonate.

Any suitable material known to the person skilled in the art may be used. Typically, glass or polystyrene is used. Polystyrene is a hydrophobic material suitable for binding negatively charged macromolecules because it normally contains few hydrophilic groups. For nucleic acids immobilized on glass slides, it is furthermore known that by increasing the hydrophobicity of the glass surface the nucleic acid immobilization may be increased. Such an enhancement may permit a relatively more densely packed formation. In addition to a coating or surface treatment with poly-L-lysine, the substrate, in particular glass, may be treated by silanation, e.g. with epoxy-silane or amino-silane or by silynation or by a treatment with polyacrylamide.

It will be evident that a tissue sample from any organism could be used in the methods of the present disclosure. The array of the present disclosure allows the capture of any nucleic acid, such as mRNA molecules, which are present in cells of a sample and are capable of transcription and/or translation. The arrays and methods of the present disclosure are particularly suitable for isolating and analyzing the transcriptome of cells within a sample, wherein spatial resolution of the transcriptomes is desirable, such as where the cells are interconnected or in contact directly with a plurality of cells. However, it will be apparent to a person of skill in the art that the methods of the present disclosure may also be useful for the analysis of the transcriptome of different cells or cell types within a sample even if said cells do not interact directly, such as a blood sample. In other words, the cells do not need to present in the context of a tissue and can be applied to the array as single cells (e.g. cells isolated from a non-fixed tissue). Such single cells, while not necessarily fixed to a certain position in a tissue, are nonetheless applied to a certain position on the array and can be individually identified. Thus, in the context of analyzing cells that do not interact directly, or are not present in a tissue context, the spatial properties of the described methods may be applied to obtaining or retrieving unique or independent spatial transcriptome information from individual cells. The disclosure relates to a method of identifying spatial expression of a nucleic acid or protein in a sample comprising identifying an interaction or binding event between a primer and/or an endogenous nucleic acid in the sample.

The sample may be a harvested or biopsied tissue sample, or possibly a cultured sample. Representative samples include clinical samples, such as whole blood or blood-derived products, blood cells, tissues, biopsies, or cultured tissues or cells, including cell suspensions. Artificial tissues may for example be prepared from cell suspension (including for example blood cells). Cells may be captured in a matrix (for example a gel matrix such as agar, agarose, etc.) and may then be sectioned in a conventional way. Such procedures are known in the art in the context of immunohistochemistry (see e.g. Andersson et al 2006, J. Histochem. Cytochem. 54(12): 1413-23. Epub 2006 Sep. 6).

The mode of tissue preparation and how the resulting sample is handled may affect the transcriptomic analysis of the methods of the present disclosure. Moreover, various tissue samples will have different physical characteristics and it is well within the skill of a person in the art to perform the necessary manipulations to yield a tissue sample for use with the methods of the present disclosure. However, it is evident from the disclosures herein that any method of sample preparation may be used to obtain a tissue sample that is suitable for use in the methods of the present disclosure. For instance, any layer of cells with a thickness of approximately 1 cell or less may be used in the methods of the present disclosure. In some embodiments, the thickness of the tissue sample may be less than about 0.9, 0.8, 0.7, 0.6, 0.5, 0.4, 0.3, 0.2 or 0.1 of the cross-section of a cell. However, since as noted above, the present disclosure is not limited to single cell resolution and hence it is not a requirement that the tissue sample has a thickness of one cell diameter or less; thicker tissue samples may if desired be used. For example, cryostat sections may be used, which may be from about 10 to about 50 µm thick. In some embodiments, the sample is about 5 µm thick. In some embodiments, the sample is about 10 µm thick. In some embodiments, the sample is about 20 µm thick. In some embodiments, the sample is about 30 µm thick. In some embodiments, the sample is about 40 µm thick. In some embodiments, the sample is about 50 µm thick. In some embodiments, the sample is about 60 µm thick. In some embodiments, the sample is about 70 µm thick. In some embodiments, the sample is about 80 µm thick. In some embodiments, the sample is about 90 µm thick. In some embodiments, the sample is about 100 µm thick.

The tissue sample may be prepared in any convenient or desired way and the present disclosure is not restricted to any particular type of tissue preparation. Fresh, frozen, fixed or unfixed tissues may be used. Any desired convenient procedure may be used for fixing or embedding the tissue sample, as described and known in the art. Thus, any known fixatives or embedding materials may be used.

In one representative example of a tissue sample for use in the present disclosure, the tissue may be prepared by deep freezing at temperature suitable to maintain or preserve the integrity (i.e. the physical characteristics) of the tissue structure, such as less than about -20° C., -25° C., -30° C., -40° C., -50° C., -60° C., -70° C. or -80° C. The frozen tissue sample may be sectioned, i.e. thinly sliced, onto the array surface by any suitable means. For example, the tissue sample may be prepared using a chilled microtome, a cryostat, set at a temperature suitable to maintain both the structural integrity of the tissue sample and the chemical properties of the nucleic acids in the sample, such as to less than about -15° C., -20° C. or -25° C. Thus, the sample should be treated so as to minimize the degeneration or degradation of the nucleic acids, such as mRNA in the tissue. Such conditions are well-established in the art and the extent of any degradation may be monitored through nucleic acid extraction, for example, total RNA extraction and subsequent quality analysis at various stages of the preparation of the tissue sample.

In another representative example, the tissue may be prepared using standard methods of formalin-fixation and paraffin-embedding (FFPE), which are well-established in the art. Following fixation of the tissue sample and embedding in a paraffin or resin block, the tissue samples may sectioned, i.e. thinly sliced, onto the array. As noted above, other fixatives and/or embedding materials can be used.

It will be apparent that the tissue sample section will need to be treated to remove the embedding material, such as to deparaffinize to remove the paraffin or resin, from the sample prior to carrying out the methods of the present disclosure. This may be achieved by any suitable method and the removal of paraffin or resin or other material from tissue samples is well established in the art, such as by incubating the sample (on the surface of the array) in an appropriate solvent, for example xylene, followed by an ethanol rinse, such as about 99.5% ethanol for about 2 minutes, about 96% ethanol for about 2 minutes, and about 70% ethanol for about 2 minutes.

The thickness of the tissue sample section for use in the methods of the present disclosure may be dependent on the method used to prepare the sample and the physical characteristics of the tissue. Thus, any suitable section thickness may be used in the methods of the present disclosure. In some embodiments, the thickness of the tissue sample section may be at least about 0.1 µm, 0.2 µm, 0.3 µm, 0.4 µm, 0.5 µm, 0.7 µm, 1.0 µm, 1.5 µm, 2 µm, 3 µm, 4 µm, 5 µm, 6 µm, 7 µm, 8 µm, 9 µm or 10 µm.In other embodiments, the thickness of the tissue sample section is at least about 10 µm, 11 µm, 12 µm, 13 µm, 14 µm, 15 µm, 20 µm, 25 µm, 30 µm, 35 µm, 40 µm, 45 µm or 50 µm.However, these are representative values only. Thicker samples may be used if desired or convenient, such as about 70 µm or 100 µm or more. Typically, the thickness of the tissue sample section is from about 1 to about 100 µm, from about 1 to about 50 µm, from about 1 to about 30 µm, from about 1 to about 25 µm, from about 1 to about 20 µm, from about 1 to about 15 µm, from about 1 to about 10 µm, from about 2 to about 8 µm, from about 3 to about 7 µm or from about 4 to about 6 µm, but as mentioned above thicker samples may be used.

In order to correlate the sequence analysis or transcriptome information obtained from each microwell of the array with the region (i.e. an area or cell) of the tissue sample, the tissue sample is oriented in relation to the microwells on the array. In other words, the tissue sample is placed on the array such that the position of a spatial index primer on the array may be correlated with a position in the tissue sample. Thus, it may be identified where in the tissue sample the position of each species of spatial index primer (or each microwell of the array) corresponds. In other words, it may be identified to which location in the tissue sample the position of each species of spatial index primer corresponds. This may be done by virtue of positional markers present on the array, as described below. Conveniently, but not necessarily, the tissue sample may be imaged following its contact with the array. This may be performed before or after the nucleic acids of the tissue sample is processed, such as before or after the cDNA generation step of the method, in particular the step of generating the first strand cDNA by reverse transcription. In some embodiments, the tissue sample is imaged prior to the reverse transcription step. In other embodiments, the tissue sample is imaged after the nucleic acids of the tissue sample have been processed, such as after the reverse transcription step. Generally speaking, imaging may take place at any time after contacting the tissue sample with the array, but before any step which degrades or removes the tissue sample. As noted above, this may depend on the tissue sample.

Advantageously, the array may comprise markers to facilitate the orientation of the tissue sample or the image thereof in relation to the microwells of the array. Any suitable means for marking the array may be used such that they are detectable when the tissue sample is imaged. For instance, a molecule, such as a fluorescent molecule, that generates a signal, preferably a visible signal, may be immobilized directly or indirectly on the surface of the array. In some embodiments therefore, the array may comprise at least two markers in distinct positions on the surface of the array. In other embodiments, more than two markers, such as at least about 3, 4, 5, 6, 7, 8, 9, 10, 12, 15, 20, 30, 40, 50, 60, 70, 80, 90 or 100 markers, can also be used. Conveniently several hundred or even several thousand markers may be used. The markers may be provided in a pattern, for example make up an outer edge of the array, such as an entire outer row of the microwells of an array. Other informative patterns may be used, such as lines sectioning the array. This may facilitate aligning an image of the tissue sample to an array, or indeed generally in correlating the microwells of the array to the tissue sample. Thus, the marker may be an immobilized molecule to which a signal giving molecule may interact to generate a signal. In some embodiments, the marker may be detected using the same imaging conditions used to visualize the tissue sample.

The tissue sample may be imaged using any convenient histological means known in the art, such as light, bright field, dark field, phase contrast, fluorescence, reflection, interference, confocal microscopy or a combination thereof. Typically, the tissue sample is stained prior to visualization to provide contrast between the different regions, such as cells, of the tissue sample. The type of stain used will be dependent on the type of tissue and the region of the cells to be stained. Such staining protocols are known in the art. In some embodiments, more than one stain may be used to visualize (image) different aspects of the tissue sample, such as different regions of the tissue sample, specific cell structures (e.g. organelles) or different cell types. In other embodiments, the tissue sample may be visualized or imaged without staining the sample, such as if the tissue sample contains already pigments that provide sufficient contrast or if particular forms of microscopy are used. In some embodiments, the tissue sample is visualized or imaged using fluorescence microscopy.

In some embodiments, a gasket sheet is used to seal the tissue sample onto the array following the step of contacting the array with the tissue sample. The use of a gasket sheet further provides force sufficient to allow cells in the tissue sample to drop into the microwells of the array. Depending on the dimension of the microwells in the array, different amount of cells will be forced into each individual microwell. In some embodiments, each individual microwell of the array comprises from about 1 to about 100 cells. In some embodiments, each individual microwell of the array comprises from about 1 to about 90 cells. In some embodiments, each individual microwell of the array comprises from about 1 to about 80 cells. In some embodiments, each individual microwell of the array comprises from about 1 to about 70 cells. In some embodiments, each individual microwell of the array comprises from about 1 to about 60 cells. In some embodiments, each individual microwell of the array comprises from about 1 to about 50 cells. In some embodiments, each individual microwell of the array comprises from about 1 to about 40 cells. In some embodiments, each individual microwell of the array comprises from about 1 to about 30 cells. In some embodiments, each individual microwell of the array comprises from about 1 to about 20 cells. In some embodiments, each individual microwell of the array comprises from about 1 to about 10 cells. In some embodiments, each individual microwell of the array comprises from about 1 to about 5 cells. In some embodiments, each individual microwell of the array comprises from about 5 to about 10 cells.

In some embodiments, each individual microwell of the array comprises an average of about 50 cells. In some embodiments, each individual microwell of the array comprises an average of about 40 cells. In some embodiments, each individual microwell of the array comprises an average of about 30 cells. In some embodiments, each individual microwell of the array comprises an average of about 20 cells. In some embodiments, each individual microwell of the array comprises an average of about 15 cells. In some embodiments, each individual microwell of the array comprises an average of about 10 cells. In some embodiments, each individual microwell of the array comprises an average of about 9 cells. In some embodiments, each individual microwell of the array comprises an average of about 8 cells. In some embodiments, each individual microwell of the array comprises an average of about 7 cells. In some embodiments, each individual microwell of the array comprises an average of about 6 cells. In some embodiments, each individual microwell of the array comprises an average of about 5 cells. In some embodiments, each individual microwell of the array comprises an average of less than about 5 cells.

Following the step of contacting the array with a tissue sample and allowing the cells to fall into the microwells, under conditions sufficient to allow hybridization to occur between the nucleic acids, such as mRNAs, of the tissue sample to the spatial index primers, the step of securing (acquiring) the hybridized nucleic acids takes place. Securing or acquiring the captured nucleic acid involves a covalent attachment of a complementary strand of the hybridized nucleic acid to the spatial index primer (i.e. via a nucleotide bond, a phosphodiester bond between juxtaposed 3′-hydroxyl and 5′-phosphate termini of two immediately adjacent nucleotides), thereby tagging or marking the captured nucleic acid with the spatial barcode domain specific to the microwell on which the nucleic acid is captured.

In some embodiments, securing the hybridized nucleic acid, such as a single stranded nucleic acid, may involve extending the spatial index primer to produce a copy of the captured nucleic acid, such as generating cDNA from the captured (hybridized) RNA. It will be understood that this refers to the synthesis of a complementary strand of the hybridized nucleic acid, such as generating cDNA based on the captured RNA template (the RNA hybridized to the capture domain of the spatial index primer). Thus, in an initial step of extending the spatial index primer, i.e. the cDNA generation, the captured (hybridized) nucleic acid, such as RNA, acts as a template for the extension in a reverse transcription step.

Reverse transcription concerns the step of synthesizing cDNA from RNA, preferably mRNA (messenger RNA), by reverse transcriptase. Thus, cDNA can be considered to be a copy of the RNA present in a cell at the time at which the tissue sample was taken, i.e. it represents all or some of the genes that were expressed in that cell at the time of isolation.

The spatial index primer, specifically the capture domain of the spatial index primer, acts as a primer for producing the complementary strand of the nucleic acid hybridized to the spatial index primer, e.g., a primer for reverse transcription. Hence, the nucleic acid, such as cDNA, molecules generated by the extension reaction (reverse transcription reaction), incorporate the sequence of the spatial index primer, i.e. the extension reaction (reverse transcription reaction) may be seen as a way of labelling indirectly the nucleic acid, such as transcripts, of the tissue sample that are in contact with each microwell of the array. As mentioned above, each species of spatial index primer comprises a spatial barcode domain (microwell identification tag) that represents a unique sequence for each microwell of the array. Thus, all of the nucleic acid, such as cDNA, molecules synthesized at a specific microwell will comprise the same nucleic acid “tag.”

cDNA molecules synthesized at each microwell of the array may represent the genes expressed from the region or area of the tissue sample in contact with that microwell, such as a tissue or cell type or group or sub-group thereof, and may further represent genes expressed under specific conditions, such as at a particular time, in a specific environment, at a stage of development or in response to stimulus etc. Thus, the cDNA at any single microwell may represent the genes expressed in a single cell, or if the microwell is in contact with the sample at a cell junction, the cDNA may represent the genes expressed in more than one cell. Similarly, if a single cell is in contact with multiple microwells, then each microwell may represent a proportion of the genes expressed in that cell.

The step of extending the spatial index primer, i.e. reverse transcription, may be performed using any suitable enzymes and protocol of which many exist in the art, as described in detail below. However, it will be evident that it is not necessary to provide a primer for the synthesis of the first cDNA strand because the capture domain of the spatial index primer acts as the primer for the reverse transcription.

After the first cDNA strand is synthesized, the cells in the array are pooled using any methods known in the art, such as centrifugation. However, the force of the centrifugation, or any other method used to collect the cells, should be such that the integrity of each cell be preserved. The cells thus collected are then sorted into one or a plurality of multiwell plates as described herein elsewhere for a secondary tagging. Typically, more than one cell are sorted into one single well of the multiwell plate. In some embodiments, at least about two cells are sorted into the same well. In other embodiments, more than two cells, such as at least about 3, 4, 5, 6, 7, 8, 9, 10, 12, 15, 20, 30, 40, 50, 60, 70, 80, 90 or 100 cells are sorted into the same well. In some embodiments, each well of the multiwell plate contains from about 2 to about 100, from about 5 to about 80, from about 10 to about 60 or from about 25 to about 50 cells. In some embodiments, each well of the multiwell plate individually contains about 5 cells. In some embodiments, each well of the multiwell plate individually contains about 10 cells. In some embodiments, each well of the multiwell plate individually contains about 15 cells. In some embodiments, each well of the multiwell plate individually contains about 20 cells. In some embodiments, each well of the multiwell plate individually contains about 25 cells. In some embodiments, each well of the multiwell plate individually contains about 30 cells. In some embodiments, each well of the multiwell plate individually contains about 35 cells. In some embodiments, each well of the multiwell plate individually contains about 40 cells. In some embodiments, each well of the multiwell plate individually contains about 45 cells. In some embodiments, each well of the multiwell plate individually contains about 50 cells. However, the number of cells contained in each well of the multiwell plate does not have to be the same. As described above, each well of the multiwell plate comprises a specific cellular index primer with a cellular barcode domain, which tags the cells located in the same well with a sequence unique to that well.

The cells may be sorted into the one or plurality of multiwell plates by any methods known in the art, such as FACS (fluorescent activated cell sorting) and MACS (magnetic activated cell sorting). Methods other than FACS and MACS may also be used. In some embodiments, the cells are sorted using FACS. In other embodiments, the cells are sorted using MACS.

Once the cells are sorted into the multiwell plate, a method of the disclosure comprises a step of second strand cDNA synthesis. In some embodiments, the cDNA synthesis takes place in situ on the plate. In some embodiments, second strand cDNA synthesis may use a method of template switching, such as using the SMART™ technology from Clontech®. SMART (Switching Mechanism at 5′ End of RNA Template) technology is well established in the art and is based on the discovery that reverse transcriptase enzymes, such as Superscript® II (Invitrogen), are capable of adding one, two, three or more nucleotides at the 3′ end of an extended cDNA molecule, i.e. to produce a DNA/RNA hybrid with a single stranded DNA overhang at the 3′ end. In some embodiments, the overhang is 1, 2, 3, 4, 5, 6, 7, 8, 9, 10, 11, 12 or more nucleotides in length. The DNA overhang may provide a target sequence to which an oligonucleotide probe can hybridize to provide an additional template for further extension and/or amplification of the cDNA molecule. Advantageously, the oligonucleotide probe that hybridizes to the cDNA overhang contains an amplification domain sequence, the complement of which can be found in the cellular index primer. This way, the resultant cDNA molecules may be further amplified and enriched using the cellular index primers while, at the same time, being tagged with a second unique, well-specific barcode (i.e. cellular barcode). This method avoids the need to ligate adaptors to the 3′ end of the cDNA first strand. Whilst template switching was originally developed for full-length mRNAs, which have a 5′ cap structure, it has since been demonstrated to work equally well with truncated mRNAs without the cap structure. Thus, template switching may be used in the methods of the present disclosure to generate full length and/or partial or truncated cDNA molecules. In some embodiments therefore, the second strand synthesis may utilize, or be achieved by, template switching.

Following the reverse transcription, the cDNA molecules are enhanced, enriched and/or amplified using cellular index primers. As discussed above, each cellular index primer comprises a cellular barcode domain comprising a nucleotide sequence that is unique to each well of the multiwell plate. Thus, all the cDNAs located in one particular well of the plate are tagged with the same nucleotide sequence corresponding to the unique cellular barcode domain. Conditions for performing such PCR amplifications are well known in the art.

It will be apparent from the above description that the cDNA molecules from a single array that have been synthesized by the methods of the present disclosure may all comprise the same annealing domain that is recognized by a first sequencing primer and the same annealing domain that is recognized by a second sequencing primer. Consequently, the cDNA molecules can be massively quantified and analyzed using any sequencing platforms known in the art, such as any next generation sequencing technologies. In some embodiments therefore, the cDNA molecules are quantified and analyzed using Illumina sequencing by first generating Illumina sequencing compatible libraries by tagmentation followed by PCR amplification. Amplifiable fragments will preferably contain both barcode domains (i.e. spatial barcode domain and cellular barcode domain) added during cDNA preparation.

The step of sequence analysis will identify or reveal a portion of captured RNA sequence and the sequences of both barcode domains (i.e. spatial barcode domain and cellular barcode domain). The sequence of the spatial barcode domain will identify the microwell to which the mRNA molecule was captured. The sequence of the captured RNA molecule may be compared with a sequence database of the organism from which the sample originated to determine the gene to which it corresponds. By determining which region of the tissue sample was in contact with the microwell, it is possible to determine which region of the tissue sample was expressing said gene. As it is possible that a given region of the tissue sample in contact with a given microwell may contain more than one cell, the sequence of the cellular barcode domain will allow differentiating captured RNA molecules with the same spatial barcode domain at the cellular level. This analysis may be achieved for all of the cDNA molecules generated by the methods of the present disclosure yielding a spatial transcriptome of the tissue sample in a single-cell fashion.

By way of a representative example, sequencing data may be analyzed to sort the sequences into specific species of spatial index primer, i.e. according to the sequence of the spatial barcode domain. This may be achieved by using, for example, the FastX toolkit FASTQ Barcode splitter tool to sort the sequences into individual files for the respective spatial index primer’s spatial barcode domain sequence. The sequences of each species, i.e. from each microwell, may be analyzed to determine the identity of the transcripts. For instance, the sequences may be identified using Blastn software, to compare the sequences to one or more genome databases, such as the database for the organism from which the tissue sample was obtained. The identity of the database sequence with the greatest similarity to the sequence generated by the methods of the present disclosure will be assigned to that sequence. In general, only hits with a certainty of at least about 1e-6, about 1e-7, about 1e-8, or about 1e-9 will be considered to have been successfully identified.

It will be apparent that any nucleic acid sequencing method may be utilized in the methods of the present disclosure. However, the so-called “next generation sequencing” techniques will find particular utility in the present disclosure. High-throughput sequencing is particularly useful in the methods of the present disclosure because it enables a large number of nucleic acids to be partially sequenced in a very short period of time. In view of the recent explosion in the number of fully or partially sequenced genomes, it is not essential to sequence the full length of the generated cDNA molecules to determine the gene to which each molecule corresponds. For example, the first about 100 nucleotides from each end of the cDNA molecules should be sufficient to identify both the microwell to which the mRNA was captured (i.e. its location on the array) at the cellular level and the gene expressed.

As a representative example, the sequencing reaction may be based on reversible dye-terminators, such as used in the Illumina™ technology. For example, DNA molecules are first attached to primers on, for example, a glass or silicon slide and amplified so that local clonal colonies are formed (bridge amplification). Four types of ddNTPs are added, and non-incorporated nucleotides are washed away. Unlike pyrosequencing, the DNA can only be extended one nucleotide at a time. A camera takes images of the fluorescently labelled nucleotides then the dye along with the terminal 3′ blocker is chemically removed from the DNA, allowing a next cycle. This may be repeated until the required sequence data is obtained. Using this technology, thousands of nucleic acids may be sequenced simultaneously on a single slide.

Other high-throughput sequencing techniques may be equally suitable for the methods of the present disclosure, e.g. pyrosequencing. In this method, the DNA is amplified inside water droplets in an oil solution (emulsion PCR), with each droplet containing a single DNA template attached to a single primer-coated bead that then forms a clonal colony. The sequencing machine contains many picoliter-volume wells each containing a single bead and sequencing enzymes. Pyrosequencing uses luciferase to generate light for detection of the individual nucleotides added to the nascent DNA and the combined data are used to generate sequence read-outs.

It is clear that future sequencing formats are slowly being made available, and with shorter run times as one of the main features of those platforms, it will be evident that other sequencing technologies will be useful in the methods of the present disclosure.

An essential feature of the present disclosure, as described above, is any method disclosed herein comprising a step of securing a complementary strand of the captured RNA molecules to the spatial index primer by, for example, reverse transcribing the captured RNA molecules. The reverse transcription reaction is well known in the art and in representative reverse transcription reactions, the reaction mixture includes a reverse transcriptase, dNTPs and a suitable buffer. The reaction mixture may comprise other components, such as RNase inhibitor(s). The primers and template are the capture domain of the spatial index primer and the captured RNA molecules are described above. In the subject methods, each dNTP will typically be present in an amount ranging from about 10 to about 5000 µM, usually from about 20 to about 1000 µM.

The desired reverse transcriptase activity may be provided by one or more distinct enzymes, wherein suitable examples are: M-MLV, MuLV, AMV, HIV, ArrayScript™, MultiScribe™, ThermoScript™, and SuperScript® I, II, and III enzymes.

The reverse transcriptase reaction may be carried out at any suitable temperature, which will be dependent on the properties of the enzyme. Typically, reverse transcriptase reactions are performed between about 37 to about 55° C., although temperatures outside of this range may also be appropriate. The reaction time may be as little as about 1, 2, 3, 4 or 5 minutes or as much as about 48 hours. Typically, the reaction will be carried out for between about 5 to about 120 minutes, such as from about 5 to about 60 minutes, from about 5 to about 45 minutes, from about 5 to about 30 minutes, from about 1 to about 10 minutes, or from about 1 to about 5 minutes according to choice. The reaction time is not critical and any desired reaction time may be used.

As indicated above, certain embodiments of the methods include an amplification step, where the copy number of generated cDNA molecules is increased, such as to enrich the sample to obtain a better representation of the transcripts captured from the tissue sample. The amplification may be linear or exponential, as desired, where representative amplification protocols of interest include, but are not limited to, polymerase chain reaction (PCR) and isothermal amplification, etc.

In preparing the reverse transcriptase, DNA extension or amplification reaction mixture of the steps of the subject methods, the various constituent components may be combined in any convenient order. For example, in the amplification reaction, the buffer may be combined with primer, polymerase and then template DNA, or all of the various constituent components may be combined at the same time to produce the reaction mixture.

By way of a representative example, any method of the present disclosure may comprise the following steps:
(a) contacting an array with a tissue sample, wherein the array comprises a substrate on which multiple species of spatial index primers are direcsuch that each species occupies a distinct position on the array and is oriented to have a free 3′ end, wherein each species of said spatial index primer comprises a nucleic acid molecule comprising, from 5′ to 3′:
   i) an annealing domain comprising a nucleotide sequence that is recognized by a first sequencing primer;
   ii) a spatial barcode domain comprising a nucleotide sequence that is unique to each microwell; and
   iii) a capture domain comprising a polythymidine sequence;
   such that nucleic acid sequence or sequences of the tissue sample hybridizes to said spatial index primers;
(b) imaging the tissue sample on the array;
(c) reverse transcribing the captured mRNA molecules to generate cDNA molecules;
(d) pooling cells from the array and sorting into one or more 96-well plates;
(e) lysing cells and performing second strand cDNA synthesis to incorporate a 5- PCR handle by template switching;
(f) amplifying cDNA molecules to incorporate a cellular index primer into each cDNA molecule, each cellular index primer comprises a nucleic acid molecule comprising, from 5′ to 3′ :
   i) an annealing domain comprising a nucleotide sequence that is recognized by a second sequencing primer; and
   ii) a cellular barcode domain comprising a nucleotide sequence that is unique to each well of the 96-well plate;
   and
(g) analyzing the sequence and/or position (e.g., sequencing) of the cDNA molecules.

The present disclosure includes any suitable combination of the steps in the above described methods. It will be understood that the present disclosure also encompasses variations of these methods, for example, where amplification is performed in situ on the plate. Also encompassed are methods which omit the imaging step.

The present disclosure also relates to a method of capturing mRNA from a tissue sample that is contacted with said array; or a method of determining and/or analyzing (e.g., partial or global) transcriptomes of a tissue sample, said methods comprising immobilizing multiple species of spatial index primers to an array substrate, wherein each species of said spatial index primers comprises a nucleic acid molecule, from 5′ to 3′:
i) an annealing domain comprising a nucleotide sequence that is recognized by a first sequencing primer;
ii) a spatial barcode domain comprising a nucleotide sequence that is unique to each microwell; and
iii) a capture domain comprising a polythymidine sequence.

In some embodiments, the disclosure relates to a method of producing an array of the present disclosure such that each species of spatial index primer is immobilized as a microwell on the array. In some embodiments, the disclosure relates to a method of producing an array comprising: immobilizing multiple species of spatial index primers to an array substrate, wherein each species of said spatial index primers comprises a nucleic acid molecule, from 5′ to 3′:
i) an annealing domain comprising a nucleotide sequence that is recognized by a first sequencing primer;
ii) a spatial barcode domain comprising a nucleotide sequence that is unique to each microwell; and
iii) a capture domain comprising a polythymidine sequence.

The present disclosure may further relates to method for making or producing a multiwell plate for use in determining and/or analyzing (e.g., partial or global) transcriptomes of a tissue sample, said method comprising immobilizing, directly or indirectly, multiple species of cellular index primers to a multiwell plate substrate, wherein each species of said cellular index primer comprises a nucleic acid molecule comprising, from 5′ to 3′:
i) an annealing domain comprising a nucleotide sequence that is recognized by a second sequencing primer; and
ii) a cellular barcode domain comprising a nucleotide sequence that is unique to each well of the multiwell plate.

The method of producing a multiwell plate of the present disclosure may be further defined such that each species of cellular index primer is immobilized as a well on the plate.

The method of immobilizing the spatial index primers on the array or the cellular index primers on the plate may be achieved using any suitable means as described herein. Where the spatial index primers or cellular index primers are immobilized on the array or plate, respectively, indirectly, they may be synthesized on the array or plate. For example, the spatial index primers or cellular index primers may be synthesized directly on the array or plate, respectively, using an automated dispensing system, such as Scienion sciFLEXARRAYER S3 printer.

The sequence analysis (e.g., sequencing) information obtained in step (g) may be used to obtain spatial information as to the nucleic acid in the sample at the cellular level. In other words, the sequence analysis information may provide information as to the location of the nucleic acid in the tissue sample in a single-cell fashion. This spatial information may be derived from the nature of the sequence analysis information obtained, such as from a sequence determined or identified, for example it may reveal the presence of a particular nucleic acid molecule which may itself be spatially informative in the context of the tissue sample used, and/or the spatial information (e.g. spatial localization) may be derived from the position of the tissue sample on the array, coupled with the sequence analysis information. However, as described above, spatial information may conveniently be obtained by correlating the sequence analysis data to an image of the tissue sample.

Accordingly, in some embodiments, a method of the present disclosure comprises a step of:

(h) correlating said sequence analysis information with an image of said tissue sample, wherein the tissue sample is imaged before or after step (b).

In some embodiments, the methods of the present disclosure can be used to perform chromatin sequencing, namely ATAC-seq (assay for transposase-accessible chromatin seq) at a single cell resolution. To do so, the same microwell array is used, but instead of having oligo-dT printed in the microwells, barcoded Transposase (TN5) is used, which will tag the open chromatin and allow ATAC-seq libraries to be generated.

In some embodiments, methods of the present disclosure can be used to perform TCR-seq. Because the library provided in the methods of the present disclosure is generated via template switching, full length cDNAs are generated, which makes spatial single cell TCR seq possible. To do so, single cell cDNAs are spatially barcoded. A TCR enrichment PCR is then performed with primers that binds to the variable region of the TCR alpha and beta chain. The primer has a Nextera R2 handle which allows a nested PCR to be performed to finish the seq library with an Illumina p5 primers.

In some embodiments, methods of the present disclosure can be used to perform cell-specific spatial transcriptomic profiling. This is made possible because the methods of the present disclosure include a cell sorting step in between the first barcoding and the second barcoding steps. The cells may be tagged with a cell-specific antibody during the first barcoding step and then only cells of interest are sorted for the second barcoding step.

### Systems

The disclosure further relates to a system comprising one or a plurality of arrays disclosed herein. In some embodiments, each of such arrays comprises one or a plurality of microwells, each microwell occupying a distinct position on the array and comprising any of the spatial index primers disclosed herein elsewhere. In some embodiments, each of such spatial index primers comprises a nucleic acid molecule comprising, in 5′ to 3′ orientation:
i) an annealing domain comprising a nucleotide sequence that is recognized by a first sequencing primer;
ii) a spatial barcode domain comprising a nucleotide sequence that is unique to each microwell; and
iii) a capture domain comprising a polythymidine sequence.

In some embodiments, each array of the disclosed system individually comprises at least about 10 microwells. In some embodiments, each array of the disclosed system individually comprises at least about 50 microwells. In some embodiments, each array of the disclosed system individually comprises at least about 100 microwells. In some embodiments, each array of the disclosed system individually comprises at least about 200 microwells. In some embodiments, each array of the disclosed system individually comprises at least about 500 microwells. In some embodiments, each array of the disclosed system individually comprises at least about 1000 microwells. In some embodiments, each array of the disclosed system individually comprises at least about 2000 microwells. In some embodiments, each array of the disclosed system individually comprises at least about 4000 microwells.

In some embodiments, each array of the disclosed system individually comprises at least about 16 microwells. In some embodiments, each array of the disclosed system individually comprises at least about 32 microwells. In some embodiments, each array of the disclosed system individually comprises at least about 64 microwells. In some embodiments, each array of the disclosed system individually comprises at least about 128 microwells. In some embodiments, each array of the disclosed system individually comprises at least about 256 microwells. In some embodiments, each array of the disclosed system individually comprises at least about 512 microwells. In some embodiments, each array of the disclosed system individually comprises at least about 768 microwells. In some embodiments, each array of the disclosed system individually comprises at least about 1024 microwells.

In some embodiments, each microwell in the array of the disclosed system is triangle shaped. In some embodiments, each microwell in the array of the disclosed system is square shaped. In some embodiments, each microwell in the array of the disclosed system is pentagon shaped. In some embodiments, each microwell in the array of the disclosed system is hexagon shaped. In some embodiments, each microwell in the array of the disclosed system is round shaped.

In some embodiments, each microwell in the array of the disclosed system is from about 25 µm to about 800 µm in depth. In some embodiments, each microwell in the array of the disclosed system is from about 1 µm to about 1000 µm in depth. In some embodiments, each microwell in the array of the disclosed system is from about 50 to about 500 microns in depth. In some embodiments, each microwell in the array of the disclosed system is from about 75 µm to about 250 µm in depth. In some embodiments, each microwell in the array of the disclosed system is about 5 µm, about 10 µm, about 50 µm, about 100 µm, about 150 µm, about 200 µm, about 250 µm, about 300 µm, about 350 µm, about 400 µm, about 450 µm, about 500 µm, or about 1000 µm in depth. In some embodiments, each microwell in the array of the disclosed system is about 400 microns in depth.

In some embodiments, the microwells in the array of the disclosed system are from about 50 microns to about 500 microns center-to-center spaced. In some embodiments, the microwells are about 50 microns center-to-center spaced. In some embodiments, the microwells are about 100 microns center-to-center spaced. In some embodiments, the microwells are about 150 microns center-to-center spaced. In some embodiments, the microwells are about 200 microns center-to-center spaced. In some embodiments, the microwells are about 250 microns center-to-center spaced. In some embodiments, the microwells are about 300 microns center-to-center spaced. In some embodiments, the microwells are about 350 microns center-to-center spaced. In some embodiments, the microwells are about 400 microns center-to-center spaced. In some embodiments, the microwells are about 450 microns center-to-center spaced. In some embodiments, the microwells are about 500 microns center-to-center spaced.

In some embodiments, the disclosed system further comprises one or a plurality of the multiwell plates disclosed herein. In some embodiments, each of the multiwell plates comprises one or a plurality of wells, each well occupying a distinct position on the multiwell plate and comprising any onr or plurality of the cellular index primers disclosed herein. In some embodiments, each of such cellular index primers comprises a nucleic acid molecule comprising, from 5′ to 3′ :
i) an annealing domain comprising a nucleotide sequence that is recognized by a second sequencing primer; and
ii) a cellular barcode domain comprising a nucleotide sequence that is unique to each well of the multiwell plate.

In some embodiments, each multiwell plate of the disclosed systems individually comprises about 24 wells. In some embodiments, each multiwell plate of the disclosed systems individually comprises about 48 wells. In some embodiments, each multiwell plate of the disclosed systems individually comprises about 96 wells. In some embodiments, each multiwell plate of the disclosed systems individually comprises about 192 wells. In some embodiments, each multiwell plate of the disclosed systems individually comprises about 384 wells. In some embodiments, each multiwell plate of the disclosed systems individually comprises about 768 wells.

In some embodiments, the spatial barcode domains of the disclosed systems individually comprise from about 8 to about 50 nucleotides. In some embodiments, the spatial barcode domains of the disclosed systems individually comprise from about 9 to about 40 nucleotides. In some embodiments, the spatial barcode domains of the disclosed systems individually comprise from about 10 to about 30 nucleotides. In some embodiments, the spatial barcode domains of the disclosed systems individually comprise from about 12 to about 25 nucleotides. In some embodiments, the spatial barcode domains of the disclosed systems individually comprise about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 35, about 40, about 45, or about 50 nucleotides. In some embodiments, the spatial barcode domains of the disclosed systems individually comprise about 16 nucleotides.

In some embodiments, the polythymidine sequences in the capture domain of the disclosed systems individually comprise from about 8 to about 50 deoxythymidine residues. In some embodiments, the polythymidine sequences in the capture domain of the disclosed systems individually comprise from about 9 to about 40 deoxythymidine residues. In some embodiments, the polythymidine sequences in the capture domain of the disclosed systems individually comprise from about 10 to about 30 deoxythymidine residues. In some embodiments, the polythymidine sequences in the capture domain of the disclosed systems individually comprise from about 12 to about 25 deoxythymidine residues. In some embodiments, the polythymidine sequences in the capture domain of the disclosed systems individually comprise about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 35, about 40, about 45, or about 50 deoxythymidine residues. In some embodiments, the polythymidine sequences in the capture domain of the disclosed systems individually comprise about 18 deoxythymidine residues.

In some embodiments, the cellular barcode domain of the disclosed systems individually comprise from about 8 to about 50 nucleotides. In some embodiments, the cellular barcode domains of the disclosed systems individually comprise from about 9 to about 40 nucleotides. In some embodiments, the cellular barcode domains of the disclosed systems individually comprise from about 10 to about 30 nucleotides. In some embodiments, the cellular barcode domains of the disclosed systems individually comprise from about 12 to about 25 nucleotides. In some embodiments, the cellular barcode domains of the disclosed systems individually comprise about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 35, about 40, about 45, or about 50 nucleotides. In some embodiments, the cellular barcode domains of the disclosed systems individually comprise about 16 nucleotides.

In some embodiments, the disclosed systems further comprise one or a plurality of gasket sheets. Such gasket sheets can be used to force cells in a sliced tissue to drop into the microwells of the disclosed array by placing the gasket sheet on top of the sliced tissue. Gasket sheets may be made of any known material. In some embodiments, the gasket sheets of the disclosed system are made of silicone. In some embodiments, the disclosed systems further comprise materials and reagents adapted for tissue digestion. In some embodiments, the disclosed systems further comprise materials and reagents adapted for permeabilization. In some embodiments, the disclosed systems further comprise materials and reagents adapted for reverse transcription (RT). In some embodiments, the disclosed systems are in form of a kit with instructions for suitable operational parameters in the form of a label or product insert.

Aspects and embodiments of the present disclosure will now be illustrated, by way of example, with reference to the accompanying tables and figures. Further aspects and embodiments will be apparent to those skilled in the art. All documents mentioned in this text are incorporated herein by reference in their entireties.

### EXAMPLES

### Example 1: General Overview of the Methodology

XYZeq uses a modified combinatorial indexing approach, similar to methods published as sci-RNA-seq (for single-cell combinatorial-indexing RNA-sequencing analysis; 23) and SPLiT-seq (for split-pool ligation-based transcriptome sequencing; 24) in 2017. Briefly, a 500-micron hexagonal well array is fabricated from Norland Optical Adhesive 81 (NOA81) on a generic histology slide using a Polydimethylsiloxane (PDMS) mold as a template. Each well is then spotted with spatially defined, barcoded oligo(dT)18 primers and dried down.

On day of experiment, the well array slide is spotted with a mixture of tissue digestion, permeabilization, and reverse transcription (RT) reagents, over which a fixed, frozen tissue section is overlaid. The array is clamped with a silicon gasket and placed in a slide microarray hybridization chamber (Agilent G2534A) to ensure microwell sealing during the short in-situ RT reaction. After reaction, the array slide is removed and placed in a 50-ml conical tube filled with 1X SSC buffer and 10% FCS. The tube with slide is vortexed for 15 seconds to dislodge cells from the wells and spun down for 10 minutes at 700 rcf to pellet the cells. After removing all but 1-2 ml from the 50-ml conical tube, cells are filtered through 70-micron cell strainer, stained with antibody, and 25-50 cells are sorted into 96-well plates that have 5 µl of second RT mix in the wells. At this point, the cells are lysed with the addition of DTT that is included in the second RT mix and a standard 1.5-hour reverse transcription and template switching reaction is performed at 42° C. followed by PCR, where barcoded Illumina P5 primers are used for secondary indexing. Barcoded cDNA is pooled together from all the wells into a 2-ml tube and cleaned and concentrated using Solid Phase Reversible Immobilization (SPRI) beads. The cDNA is eluted in 15 µl, quantified and checked for appropriate size distribution. Illumina compatible sequencing libraries are then generated from the cDNA by tagmentation followed by PCR, such that both combinatorial barcodes are retained on sequenced fragments.

### Example 2: Fabrication of Microwell Array Chips for XYZeq

The array fabrication for XYZeq involves positive mold design and fabrication as well as production of negative PDMS mold. For the positive mold, the microwell array was designed as a hexagonal pack of 500 µm wells (measured center to center), spaced by 10 µm. The array design included corner fiducial markers for accurate alignment and reagent dispensing by a Scienion sciFLEXARRAYER S3. A UV mask of the microwell design was obtained from CAD/Art Services (Bandon, Oregon). A 100 mm silicon wafer was spin-coated with SU-8 2150 photoresist at 2000 rpm for 30 seconds, soft-baked at 95° C. for 2 hours, UV exposed with mask for 30 minutes, post-baked at 95° C. for 20 minutes, then developed for 1 hour.

The negative PDMS mold was produced as follows. PDMS (Sylgard 184) comes in two liquid components: component A is the base and component B is the curing agent. Using a weighing scale, added 30 grams of the component A and then added component B which is 1:10 of component A into a 100-mm petri dish. Mixed the two components with a plastic swab. Placed the silicon wafer positive array mold into the dish and then degassed for 30 minutes to an hour in a vacuum desiccator until no bubbles remain. Centrifuged the dish with silicon wafer at 1000 rcf for 10 minutes to bring the wafer down to bottom and remove any remaining bubbles. Cured the PDMS in a 70° C. oven overnight. Peeled the PDMS from the wafer and then cut out the molds using a razor blade.

The microwell array chips were fabricated as follows. Heated hot plate to 100° C. Added 150 µl of NOA81 to the PDMS mold and spread it to cover the entire array. Placed a histology slide on top of the PDMS mold and place a transparent 20 g weight on top of slide. UV cured the NOA81 for 2 minutes on one side, then 1 minute on the back side without weight. Cooled briefly and then peeled the PDMS mold off the NOA81 array to complete the fabrication process.

Microwell array chips were printed with spatially barcoded oligo(dT)18 primers using a Scienion sciFLEXARRAYER S3 printer. In the particular experiment performed, the array was printed with 768 uniquely barcoded oligo(dT)18 primers. The S3 printer was housed in a chilled and humidity-controlled chamber so that during the printing process, the source plate did not evaporate. The oligos were dried in the chip and stored until day of experiment.

### Example 3: Validation of XYZeq Platform Using Cell Lines

The feasibility of XYZeq platform was validated using cell lines from two different species mixed at concentrations determined by the relative spatial location of each well. The capability of XYZeq platform to identify unique cellular populations with distinct spatial organization within the intact tissue was also validated using a murine heterotopic liver tumor model.

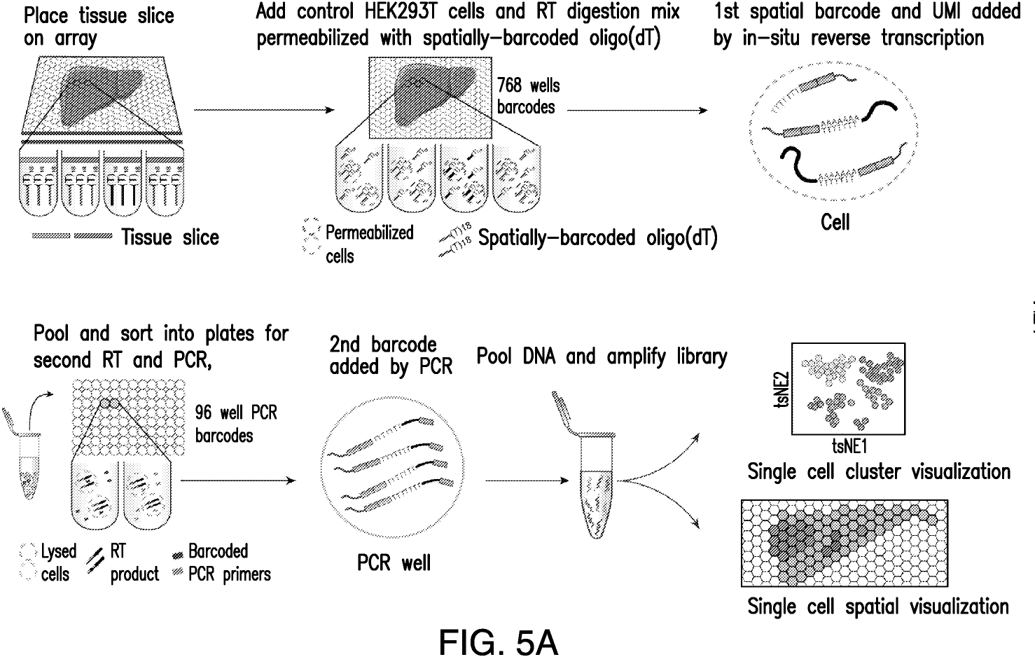

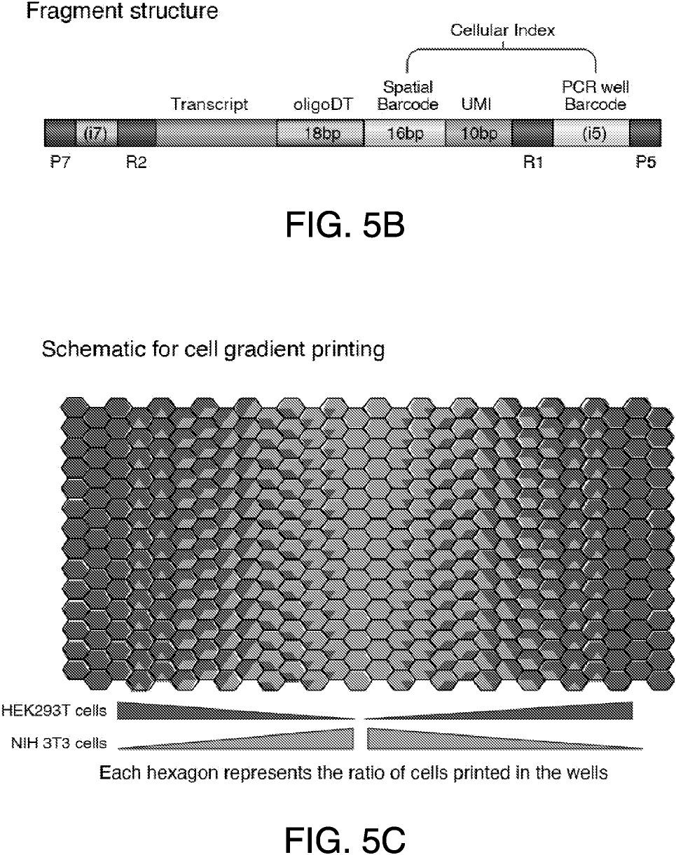

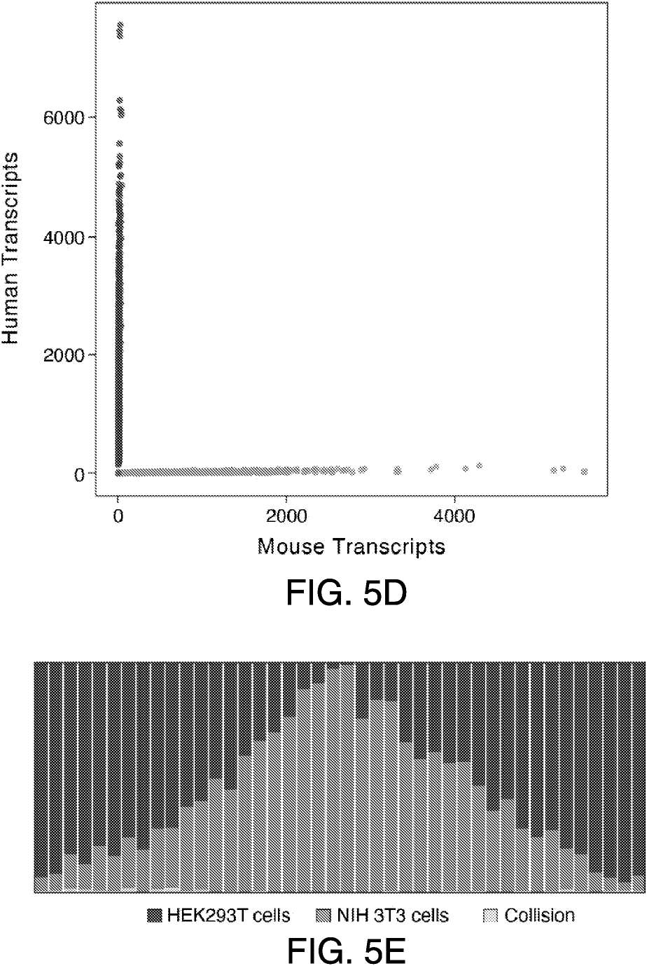

XYZeq expands on recent methods of split-pool indexing (17, 18) for single cell sequencing to enable simultaneous recording of spatial information. Cellular transcripts are spatially encoded in situ by barcoded oligos in 250 µm from center of hexagonal microwell arrays. Cells were spotted into wells, permeabilized, and indexed with well-specific barcoded oligo d(T) primers (RT-index) containing a unique molecular identifier and a PCR handle. This is followed by reverse transcription, a second round of barcoding by PCR, and tagmentation to generate single cell RNA-sequencing libraries (FIG. 5A). The combination of spatially informative RT-index and split-pool PCR-index allows us to obtain single-cell transcriptome data and simultaneously assign each cell to a specific well in the array. With two rounds of combinatorial barcoding, first with 768 positional RT-indices and second with 384 PCR-indices, up to 294,912 barcode combinations can be generated.

In order to validate that XYZeq generates interpretable single cell transcriptomes, we performed a mixed species experiment where a mixture of 80 human (HEK293T) and mouse (NIH/3T3) cells were deposited into 768 barcoded microwells at various different ratios. We demonstrate the feasibility of XYZeq using cell lines from two different species mixed at concentrations determined by the relative spatial location of each well. Each column in the microwell array had either descending or ascending concentrations of human or mouse cells that were mixed together at a gradient (FIG. 5B). The cells from the microwell chip were pooled and FACS sorted into each well of four 96-well plates at a concentration of 25 cells/well. We obtained a total of 4,871 uniquely barcoded cells where the reads were subsequently aligned to the mouse or human genome. Our data revealed a clear separation of reads between species where each cell was explicitly assigned to a single species (>90% of the reads aligned to a single genome) with only 8.4% collision rate where the cells mapped to both human and mouse, which is consistent with the expected barcode collision rate using these parameters (FIG. 5C). We obtained a median of 939 UMIs and 439 genes per human cell and 816 UMIs and 336 genes per mouse cell (FIG. 5D). Additionally, the ratio of human to mouse cells in each column was consistent with the expected ratio of cells printed on the gradient pattern (FIG. 5E). These results suggest that there was very little transfer of barcodes between wells when cells are pooled before reverse transcription and that XYZeq produces high quality scRNA-seq libraries.

### Example 4: Validation of XYZeq Platform Using Fixed Tissue Section

Whether XYZeq could generate single cell RNA-seq libraries from a fixed tissue section was next determined. This requires tissue digestion, cell permeabilization and spatial indexing in the microwells. To test this, we used a heterotopic murine tumor model that is established by intrahepatic injections of a syngeneic colon adenocarcinoma cell line, MC38, into immunocompetent mice. The MC38 was tagged with a luciferase (MC38-Luc) to permit visualization of the tumor growth in the liver to determine the correct timeframe to sac the animal. When tumors grew to approximately 5 mm in diameter by bioluminescence imaging (day 10-12 post injection), mice were sacrificed and livers bearing the tumor nodule were harvested, fixed, and frozen in the embedding matrix cartridge. We selected the liver tumor model because clear margins define the tumor/liver boundary and MC38 tumor is immunogenic (30). MC38 tumor also has immunomodulating properties with immune cells accumulating at the tumor/tissue interface. Previous data have shown ~15-20% of all cells in the tumor approximately 12 days post tumor inoculation are infiltrating immune cells (23, 24). Thus, we predicted that our XYZeq data may capture both tissue resident and infiltrating cell populations with distinct spatial organizations during disease progression.

We adapted the XYZeq platform for studies of intact tissue sections. To ensure again that transcriptomes could be assigned to discrete single cells, fixed human HEK293T cells were spotted into a barcoded microwell array at an average of 58 cells per well and then frozen at -80° C. to provide a control for detecting mixing within spatial or PCR wells. Next, a 25 µm slice of fixed frozen liver/tumor tissue from a C57BL/6 mouse was placed on top of the pre-frozen -80° C. microwell array while a sequential 10 µm slice was taken and fixed for immunohistochemical staining. An image of the tissue on the array is captured to determine the gross orientation of the tissue on the array. After imaging, the array is sealed with a silicone gasket then clamped down in an Agilent Microarray Hybridization slide chamber. The Microarray Hybridization chamber serves two purposes: 1) mechanical pressure to force the tissues into the wells and 2) to prevent evaporation during the 42° C. incubation when tissue digestion, cellular permeabilization, in situ oligo(dT) annealing, and reverse transcription (RT) were performed (FIG. 5A).

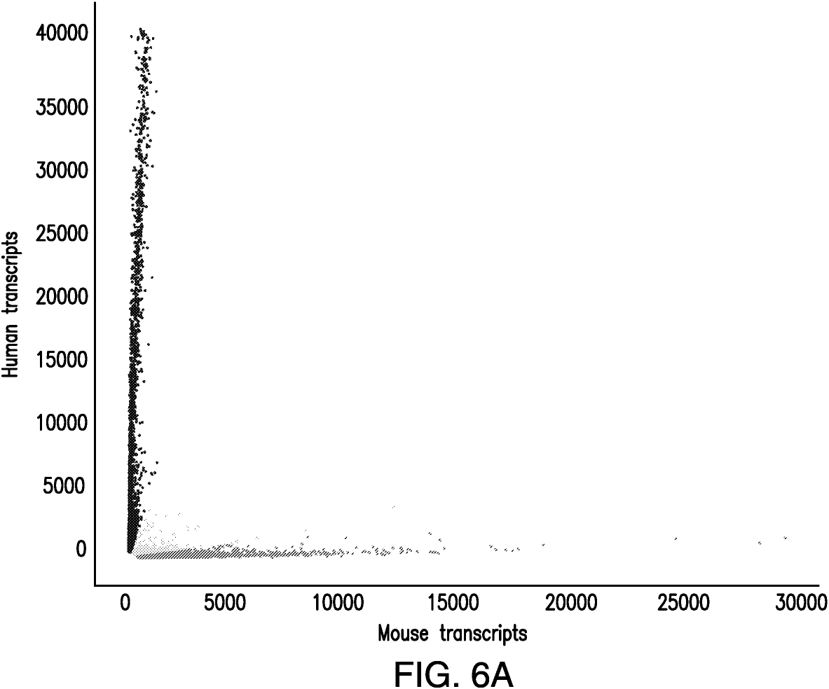

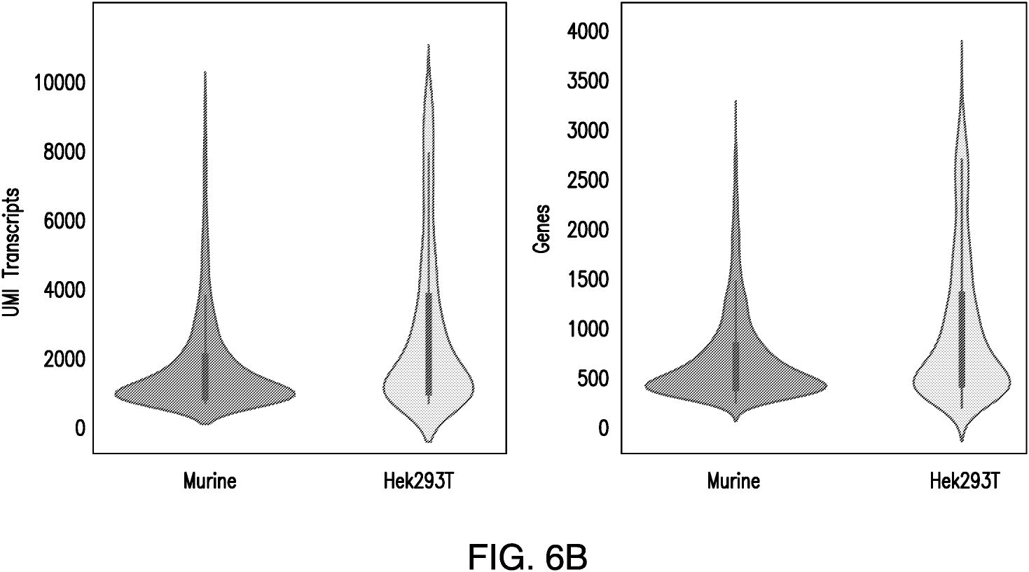

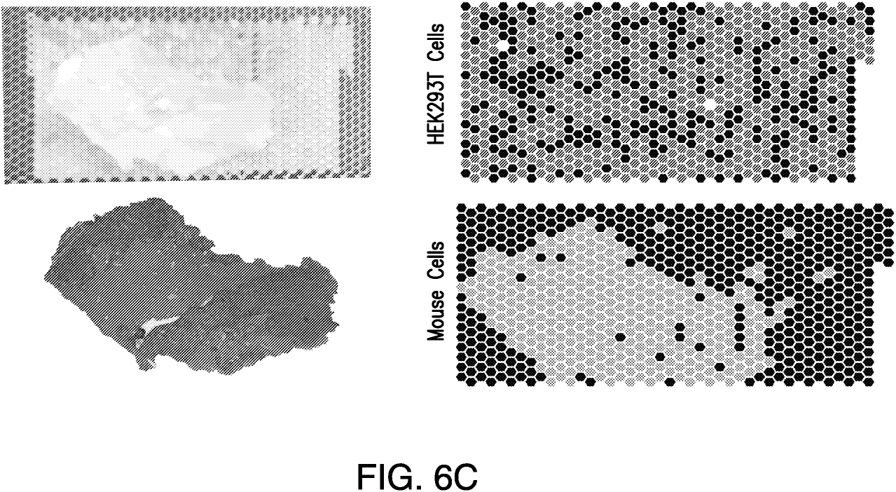

The tissue-based protocol generated data with high single cell integrity 56% of cells mapping to mouse and 34% to human with 9.6% collision rate (FIG. 6A). At a sequencing saturation of 46%, we detected a median of 1596 transcript UMIs and 629 unique genes per HEK293T cells and 1009 UMI transcripts and 456 unique genes per cell from the heterotopic murine tumor model (FIG. 6B). Image of the tissue taken from the array as well as the hemoxylin and eosin (H&E) immunohistochemical staining of the tissue reveals distinct boundaries of the tumor and liver tissue (FIG. 6C). Reconstructing the spatial arrangement of cells from the single cell data revealed human cells scattered across the entire array and mouse cells sequestered to wells that were overlaid with tissue (FIG. 6D). Importantly, these results demonstrate that XYZeq can generate spatially-resolved single cell RNA-seq data from frozen tissue.

It is important to note that, in order to achieve high quality RNA from fixed frozen tissue, the Microarray Hybridization Chamber housing the slide had to undergo gradual step-wise temperature increase from -80° C., -20° C., 4° C., 25° C. to 42° C. In the absence of this step-wise temperature change, RNA extracted from the array was severely degraded (data not shown).

### Example 3: Identification of Distinct Cell Populations Found in Liver Tumor Model

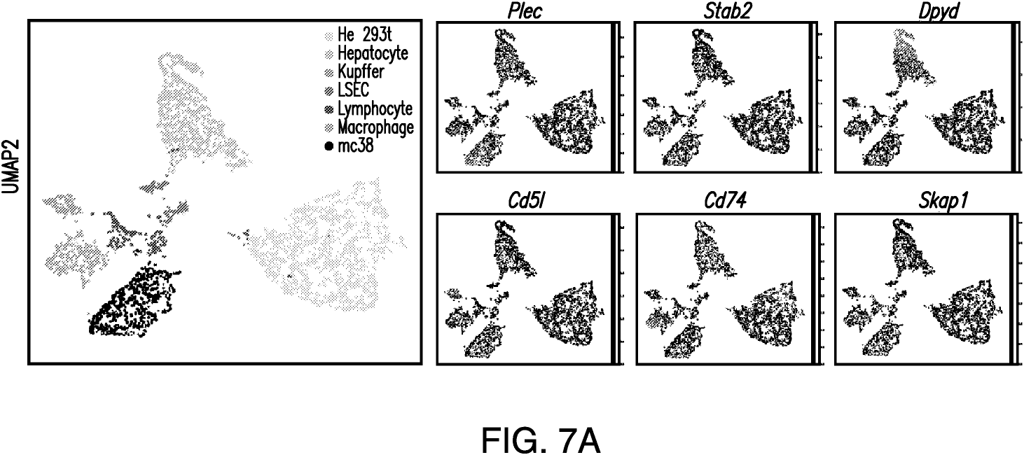

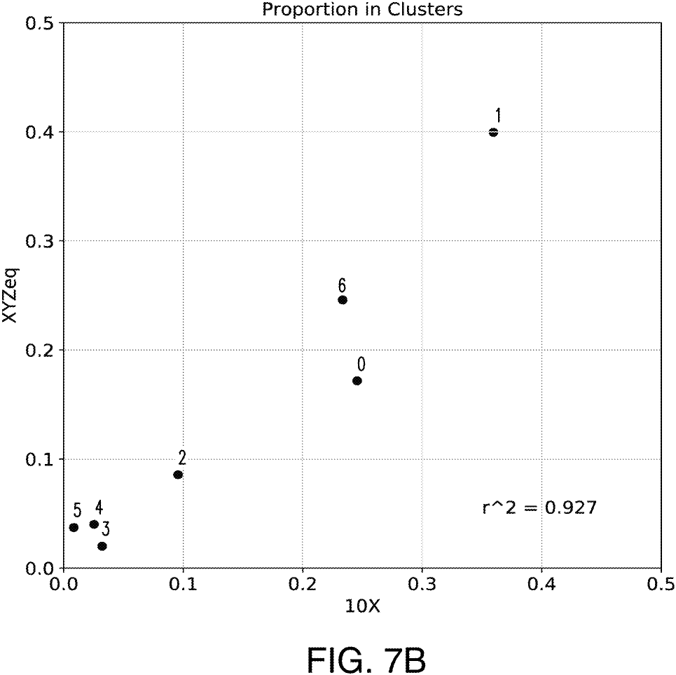

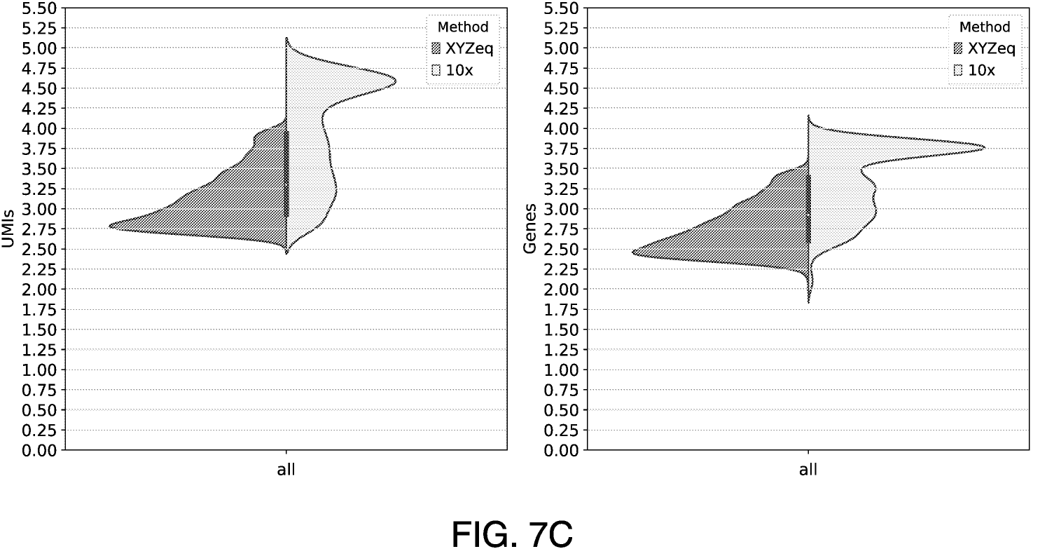

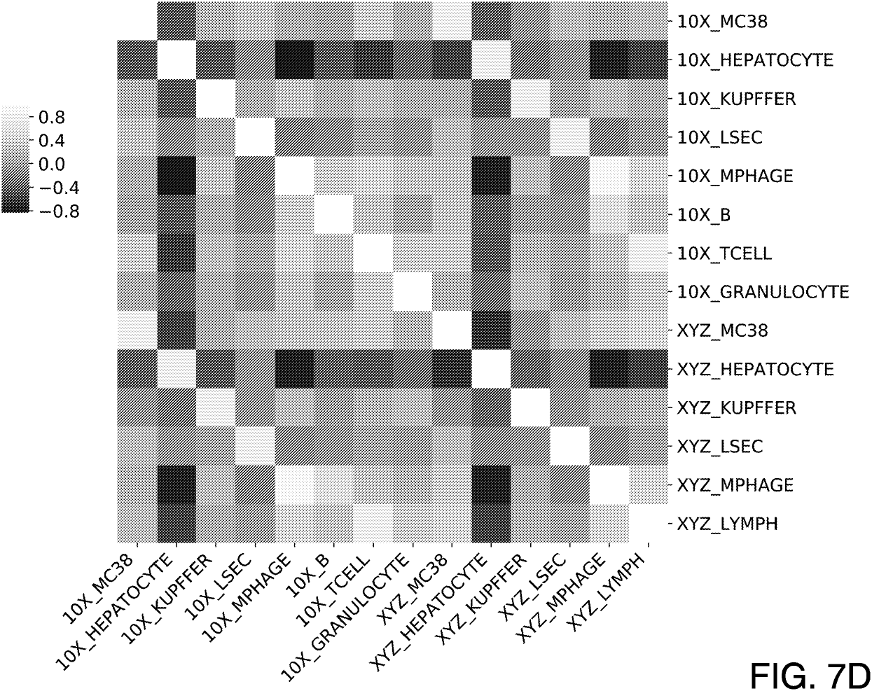

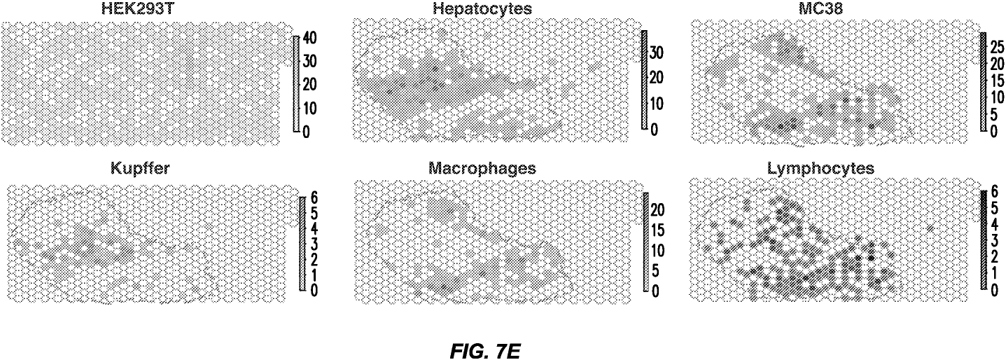

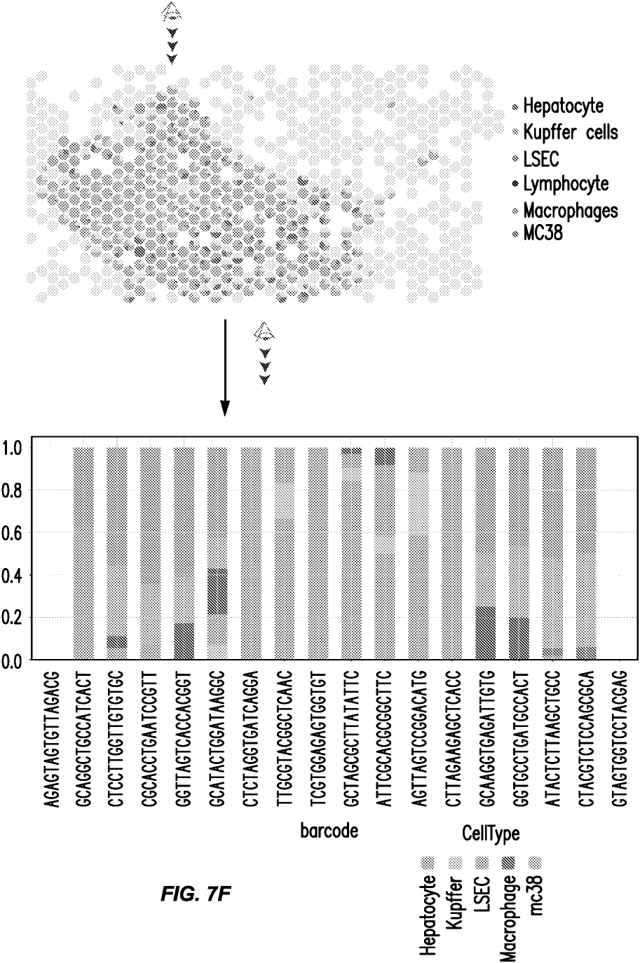

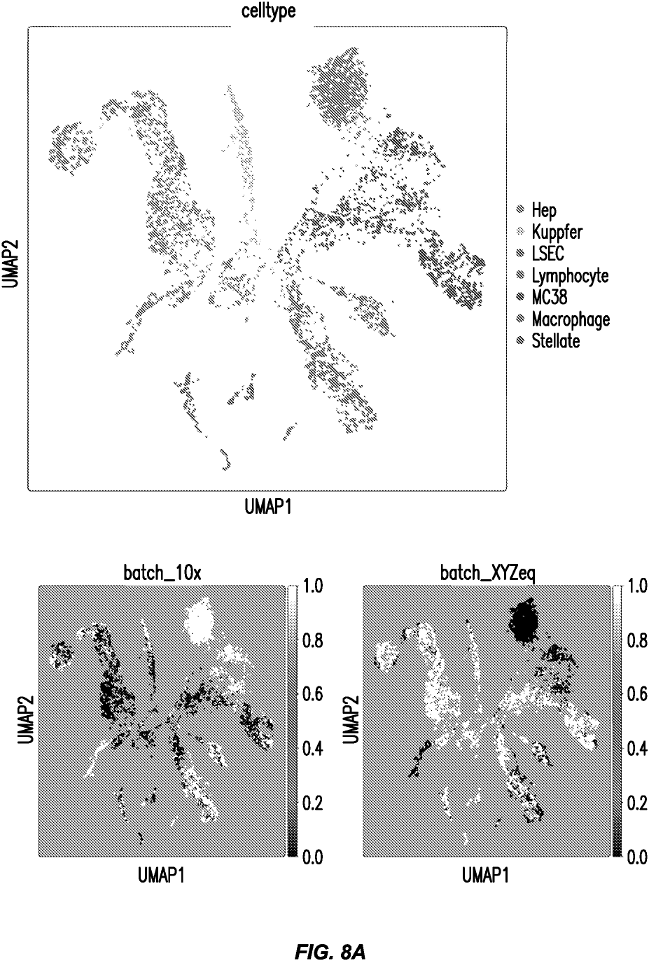

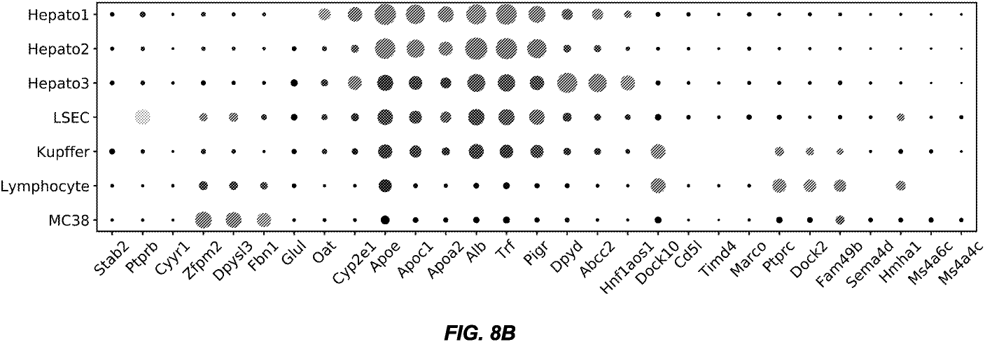

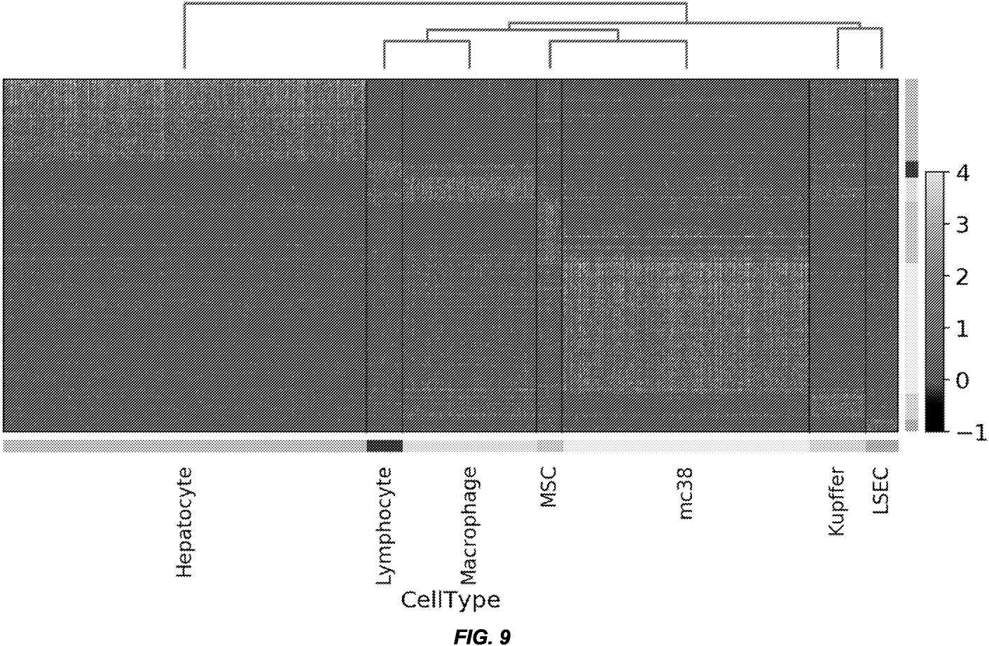

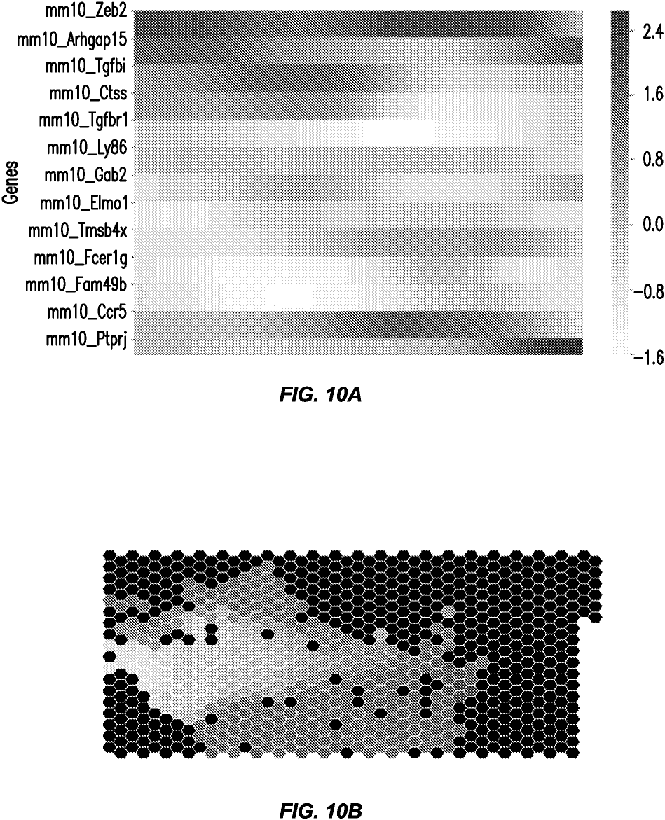

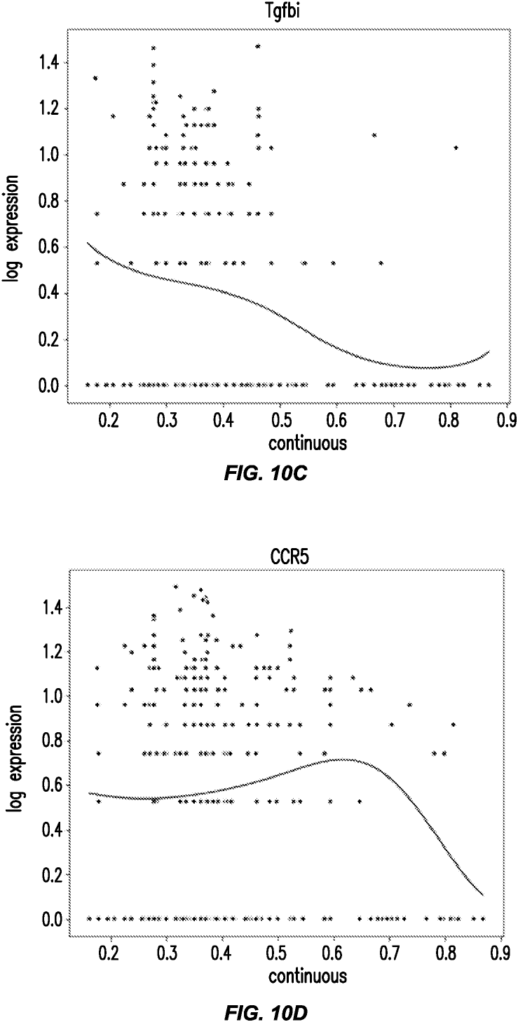

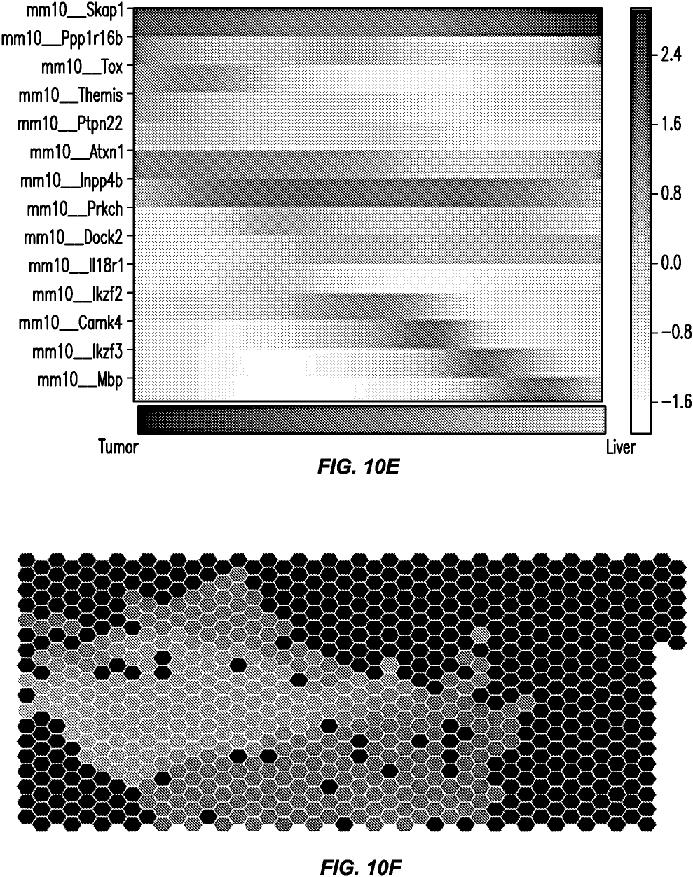

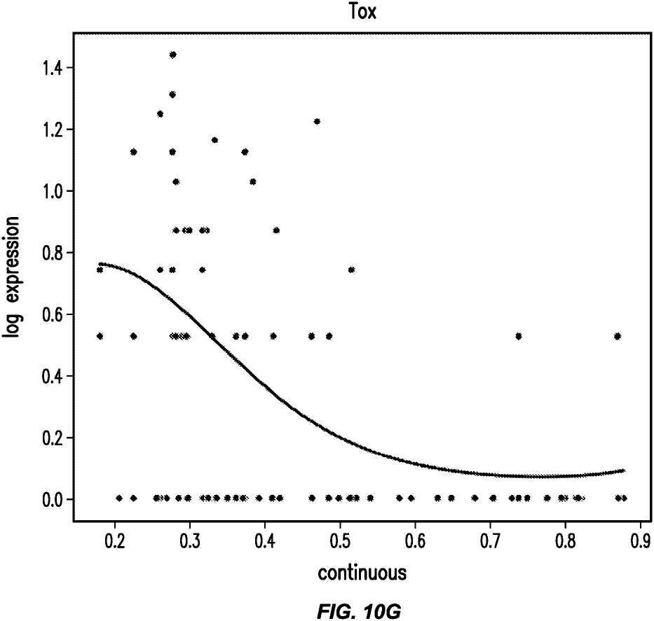

In a tissue section processed with XYZeq, we generated a total of 26,436 unique barcode combinations, with an average of 456 unique genes detected for the 4,788 barcodes expressing at least 500 UMIs which we filtered as cell containing compartments. Unsupervised Leiden clustering revealed seven distinct cell populations in our scRNAseq dataset: including HEK293T, MC38 tumor, macrophages, Kupffer cells, liver sinusoidal endothelial cells (LSEC), lymphocytes, and hepatocytes (FIG. 7A). Each cluster could be defined by a distinct gene expression profile including Plec for Mc38 tumor, Stab2 for LSECs, Dpyd for hepatocytes, Cd5l for Kupffer cells, Cd74 for macrophages, and Skap1 for lymphocytes (FIG. 7B). Using Harmony, an algorithm that can normalize datasets to integrate data from cells across multiple experiments with diverse experimental and biological factors, we were able to merge XYZeq dataset with 10X Chromium (v3) to determine how the metrics compare. Cells for 10X Chromium were processed from previously fixed, frozen, and sliced heterotropic liver tumors that were pooled together into single cell suspension and sorted prior to library generation using 10X Chromium manufacturer’s protocol. To merge the datasets, the raw counts matrices for XYZeq and 10X were filtered only for the final set of cell barcodes, while retaining all possible mouse genes, and combined into a set of 5453 cells across 22374 genes. Data were normalized to 1 million counts per cell, logged, and then scaled to a mean of zero and variance of 1, per gene. Data were preprocessed using PC, followed by Harmony. Visualization was done with UMAP and clustering was done with Leiden and a resolution of 0.2 (FIG. 8A).

To determine how well the two platforms correlated, cells were filtered for the 2500 cell barcodes expressing the most UMIs. Using the annotations from the merged dataset, the proportion of cells from each method and belonging to each cell type was calculated. Proportions for each cell type were plotted, and the coefficient of determination was calculated by fitting to the model that assumes proportions are equal between the two methods. Using this metric, correlation between the clusters from the 10X data to XYZeq was high at the r^2 value between the two different single cell platforms was 0.961, with cluster composition that was similar between the two platforms. (FIG. 7B). The median number of UMIs that were detected from the 10X Chromium (v3) was 1805 and 857 genes per cell. Conversely, the single cell metrics that were recovered from our aggregated data of 6 tissue slices were processed by XYZeq platform using fixed frozen tissue slices detected 1124 UMIs and 468 genes per cell (FIG. 7C). Comparative analysis allowed us to reveal the heterogeneity within each population that differed in gene expression profile, function, and organization. Tiling distinct expression profiles based on known representative maker genes across the 7 cell types, we were able to visualize the overlap of gene expression across the cell populations (FIG. 8B). The size of the bubble for each gene correlates to the degree of expression for the cell type.

To determine the degree of concordance between the XYZeq and 10X genomics platform, we tried to visualize via a heatmap, where we correlated the scaled gene expression between clusters generated from our assay and those generated from the 10x genomics platform (FIG. 7D). All of the clusters found in our assay correlate with all but one of the corresponding cell types found using the 10X platform, the sole exception being a small population of B cells. These cells did not separately cluster in the XYZeq data but are likely being captured, at least in part, by the lymphocyte population. Other correlations are observed among the immune cell types, and notably between the two clusters of macrophages, those marked by Cd74 and Tgfbr 1 indicating infiltration from the periphery, and the others marked by Clec4f and Timd4 suggesting they are the tissue-resident Kupffer cells of non-hematopoetic origin. These data show the high concordance between the XYZeq method and the 10X genomics platform.

### Example 4: Gene Expression Profiles of Lymphocytes Reveal Tissue Specific Adaptation

The 10X Chromium can generate a comprehensive dataset of gene expression profiles and cell types, it cannot spatially localize the cells within the context of the tissue. To determine whether XYZeq’s single cell data can faithfully reconstruct the spatial histological features of our liver tumor tissue, we explored the localization of our single cell data clusters to our spatial array. Grossly, the density heatmap of hepatocytes and tumor cells across the spatial wells overlaps the hemoxylin and eosin (H&E) immunohistochemical staining of a serial section (outlined as a gray dotted line) (FIG. 7D and FIG. 7E). Projection of other cell types revealed distinct spatial organization pattern for lymphocytes, macrophages, Kupffer, hepatocytes, MC38 and LSECs with distinct density pattern scattered throughout the array (FIG. 7E). In particular, the lymphocyte distribution overlaps with both hepatocytes and tumor, while macrophages seem to be sequestered to the tumor region. LSEC wells also overlap with the tumor and hepatocytes region while Kupffer cells, expectedly overlap only with the hepatocyte defined wells. Consistent with the enrichment of cell-type specific markers in the UMAP projection, expression of Plec spatially colocalized with tumor cells, Stab2 with lymphocytes, Dpyd with hepatocytes, Cd5l with Kupffer cells, Cd74 with macrophages, and Skap1 with LSECs (FIG. 8). However, the density spatial map revealed spatial overlap of multiple different cell types suggesting potential hotspots of cellular interaction. To quantify the composition of cells that occupy each spatial well, we utilized our single cell data to generate a well-specific pie chart that delineates the ratio of cellular subgroups that are present in each well (FIG. 7F). The pie chart-based analysis revealed a co-localization of immune cells that were enriched in the liver/tumor interface - information that would not be available in a scRNA-seq platform that dissociates tissue. Quantification of one column on the spatial array is represented as a bar plot. Similar to our visual analysis of the spatial density plot, macrophages are sequestered in tumor areas while lymphocytes are co-detected in both hepatocyte and tumor regions, suggesting distinct spatial organization occurs within intact tissue. These experiments demonstrated that XYZeq can profile single cell transcriptomes in tissue and can generate comparable metrics to other high throughput in situ based scRNAseq platforms, while mapping cells types to specific regions within the tissue microenvironment.

Spatially-resolved sequencing permits expression analysis in the context of the tissue architecture that is not possible with current single cell sequencing methods. The lack of spatial information with the methods prevents the analysis of how changes in cell state affect neighboring cells in the tissue microenvironment. XYZeq is foremost a new scRNA-seq workflow that retains spatial information, thereby allowing us to recapitulate the gross organizational layout of the tissue section for cellular proportion and heterogeneity, while also allowing us to discern the location and gene expression of each single cell residing within the tissue microenvironment. With XYZeq, we can begin to decipher the intercellular dynamics that underlie the function of normal and aberrant tissues. While FISH imaging-based methods also offer true single cell spatial resolution, they are limited in terms of throughput and the creation of custom probes. As a sequencing-based approach, XYZeq leverages the enormous technical development in the NGS field, benefiting from increased throughput and decreasing cost per data point. While it is too early to predict if spatially resolved transcriptomics will find integration into routine clinical pathology, it can at a minimum, can begin to map large scale transcriptomic data within the context of tissues and organisms.

### Example 5: Use of XYZeq for Cell-Specific Spatial Transcriptomics Profiling

XYZeq can be used to study cell-specific spatial transcriptomic profiling. To do so, at the step where RT buffer is spotted to the microwell array, antibody of interest can be added to the first RT mix. This will then allow for the antibody tagging of cells of interest be sorted. Non-limiting examples of antibodies that may be used are provided in Table 1.

TABLE 1

Examples of antibodies for use in cell-specific spatial transcriptomics profiling with XYZeq.

| | |
|---|---|
| Cell Type | Antibodies |
| 4-1BBL (CD 137L) | mouse TKS-1; mouse LOB12.3; mouse 3H3 |
| BTLA | mouse PJ196; mouse PK18.6 |
| CD11a | human R7-1 |
| CD11b | mouse M1/70 |
| CD11b | mouse M1/70 |
| CD137 (4-1BB) | mouse 3H3 |
| CD154 (CD40L) | mouse MR-1; mouse 9H10 |
| CD19 | mouse 1D3 |
| CD19 | mosue ID3 |
| CD20 | mouse AISB12 |
| CD25 (IL-2Ra) | mouse PC-61.5.3; mouse 7D4; human 7G7B6; mouse PC-61; human mA251; human 2A3 |
| CD28 | human 9.3; mouse PV-1 |
| CD3ε | mouse 145-2C11; human OKT-3 |
| CD4 | mouse GK1.5; mouse YTS177; mouse YTS191 |
| CD40 | mouse FGK4.5/FGK45; human G28.5 |
| CD44 | human Hermes-1; mouse/human IM& |
| CD45RB | mouse HB220 |
| CD80 | mouse 16-10A1; mouse GL-1 |
| CD8α | mouse 2.43; mouse 53-6.72; mouse YTS169.4; human OKT-8 |
| CSF1 | mouse 5A1 |
| CTLA-4 (CD152) | mouse 9D9; human BN13; mouse UC10-4F10-11; mouse 9h10 |
| Endothelial cell antigen | mouse MECA-32 |
| F4/80 | mouse CI:A3-1 |
| GM-CSF | mouse MP1-22E9 |
| HLA-DQ | human HLADQ1 |
| HLA-DR | Human L243 |
| ICOS | mouse 17G9; mouse 27A12 |
| ICOSL | mouse HK5.3 |
| IFNγ | mouse XMG1.2; human B27; human B133.5 |
| IL-10R | mouse 1B1.3A |
| Il-12 | mouse R1-5D9 |
| IL-12 p40 | mouse C17.8 |
| 11-17 | mouse 17F3 |
| IL-21R | mouse 4A9 |
| IL-4 Ra | mouse 11B11 |
| il10 | mouse JES5-2A5 |
| IL10 | mouse JES5-2A5 |
| Il12p70 | mouse 20C2 |
| Il1alpha | mouse ALF-161 |
| Il1beta | mouse B122 |
| LAG3 | mouse C9B7W |
| LFA-1 | mouse M17/4; human Ts-1/22.1.1.13 |
| LPAM-1 | mouse DATK32 |
| Ly6c | mouse HK1.4 |
| Ly6G (Gr-1) | mouse RB6-8C5; mouse 1A8 |
| MAdCAM-1 | mouse MECA-367 |
| NK1.1 | mouse PK136 |
| NKG2D | mouse HMG2D |
| PD-1 (CD279) | mouse J43; mouse RMP1-14; mouse 29F.1A12; human J110; human J116 |
| PD-L1 (B7-H1) | mouse 10F.9G2 |
| PD-L2 (B7-DC) | mouse Ty25 |
| TCRbeta | mouse HB218/ H57-597 |
| TGFβ | mouse/human 1D11.16.8 |
| Thy1 (CD90) | mouse M5/49.4.1; mouse T24/31 |
| Thy1.1 (cd90.1) | mouse 19E12 |
| Thy1.2 (CD90.2) | mouse 30H12 |
| thy1.2 (CD90.2) | mouse 30H12 |
| TIGIT | mouse IG9 |
| TIM-3 | mouse B8.2C12; mouse RMT3-23 |
| TNFα | mouse TN3-19.12; mouse XT3.11 |

### Example 6: Use of XYZeq for Spatial TCR-seq

First part of the library preparation is the same as described above up to the generation of cDNAs. Then this is followed by PCR amplification of TCRα and TCRβ genes by a cocktail of TCRα and TCRβ variable region primers that binds to the end of the V segment for a semi-nested PCR. A list of non-limiting exemplary multiplex primer sequences for spatial TCR-seq using XYZeq is provided in Table 2.

TABLE 2

Examples of multiplex primer sequences for spatial TCR-seq using XYZeq.

| Primer | Sequence | SEQ ID NO: |
|---|---|---|
| Trav1 | GTCTCGTGGGCTCGGAGATGTGTATAAGAGACAGCAGGGTGTGGAGCAGCCTGCCAAGTCTCGTGGGCTCGGAGATGTGTATAAGAGACAGATCTA | 1 |
| Trav2/21 | TTGGTACCGACAGGTTCCGTCTCGTGGGCTCGGAGATGTGTATAAGAGACAGGGCGA | 2 |
| Trav3 | GCAGGTGGAGCAGCGCGTCTCGTGGGCTCGGAGATGTGTATAAGAGACAGTCTGCT | 3 |
| Trav4 | CTGAGATGCAATTTTGTCTCGTGGGCTCGGAGATGTGTATAAGAGACAGCTACTT | 4 |
| Trav5 | CCCTTGGTATAAGCAAGAGTCTCGTGGGCTCGGAGATGTGTATAAGAGACAGACCCA | 5 |
| Trav6 | ACTCTKTTCTGGTATGTGTCTCGTGGGCTCGGAGATGTGTATAAGAGACAGAAGGT | 6 |
| Trav7 | ACAGCAGAGCCCAGAATCGTCTCGTGGGCTCGGAGATGTGTATAAGAGACAGCCTGA | 7 |
| Trav8 | GCATCCACGAGGGTGAAGTCTCGTGGGCTCGGAGATGTGTATAAGAGACAGAGCTG | 8 |
| Trav9 | AGATGCAASTATTCCTGTCTCGTGGGCTCGGAGATGTGTATAAGAGACAGCATGG | 9 |
| Trav10 | AGAGAAGGTCGAGCAACAGTCTCGTGGGCTCGGAGATGTGTATAAGAGACAGAAGAC | 10 |
| Trav11 | CCAAGTGGAGCAGAGTC GTCTCGTGGGCTCGGAGATGTGTATAAGAGACAGGTGAC | 11 |
| Trav12 | CCAGACAGAAGGCCTGGGTCTCGTGGGCTCGGAGATGTGTATAAGAGACAGGTCCTT | 12 |
| Trav13 | GGTTCTGCAGGAGG GTCTCGTGGGCTCGGAGATGTGTATAAGAGACAGCAGCA | 13 |
| Trav14 | GCAGGTGAGACAAAGGTCTCGTGGGCTCGGAGATGTGTATAAGAGACAGCTGGA | 14 |
| Trav15 | CTGTTCATATGAGACAAGTGTCTCGTGGGCTCGGAGATGTGTATAAGAGACAGAGAAG | 16 |
| Trav16 | GTAACACAGACTCAGACGTCTCGTGGGCTCGGAGATGTGTATAAGAGACAGCAGTC | 16 |
| Trav17 | CGTGGACCAGCCTGATGCGTCTCGTGGGCTCGGAGATGTGTATAAGAGACAGGAGCA | 17 |
| Trav18 | GAGTCCTCGGTTTCTGAGGTCTCGTGGGCTCGGAGATGTGTATAAGAGACAGCCAGC | 18 |
| Trav19 | AAGTTAAACAAAGCTCTCCGTCTCGTGGGCTCGGAGATGTGTATAAGAGACAGCCTCC | 19 |
| Trav23 | GTTTCTCGGCTCCTGGGTCTCGTGGGCTCGGAGATGTGTATAAGAGACAGGTGAC | 20 |
| Trbv01 | TTTGCTGGAGCAAAACCCGTCTCGTGGGCTCGGAGATGTGTATAAGAGACAGGACCC | 21 |
| Trbv02 | GAAAATTATCCAGAAACCGTCTCGTGGGCTCGGAGATGTGTATAAGAGACAGGGACC | 21 |
| Trbv03 | CAAAGTCTTACAGATCCCGTCTCGTGGGCTCGGAGATGTGTATAAGAGACAGGAGAC | 23 |
| Trbv04 | GGCTGTTTTCCAGACTCCGTCTCGTGGGCTCGGAGATGTGTATAAGAGACAGAACAC | 24 |
| Trbv05 | TAAAATTACTCAGTCACCGTCTCGTGGGCTCGGAGATGTGTATAAGAGACAG | 25 |
| Trbv12 | GATTCTGGGGTTGTCCAGTCTCCGTCTCGTGGGCTCGGAGATGTGTATAAGAGACAGGAGGC | 26 |
| Trbv13-1+2 | TGCAGTCACCCAAAGCCCGTCTCGTGGGCTCGGAGATGTGTATAAGAGACAGGAGGC | 27 |
| Trbv13-3 | TGCAGTCACCCAAAGTCCGTCTCGTGGGCTCGGAGATGTGTATAAGAGACAGGAAGC | 28 |
| Trbv14 | TGGAGTCACCCAGTCTCCGTCTCGTGGGCTCGGAGATGTGTATAAGAGACAGGATGC | 29 |
| Trbv15 | TGGAGTTACCCAGACACCGTCTCGTGGGCTCGGAGATGTGTATAAGAGACAGAATGC | 30 |
| Trbv16 | TGGTGTCATCCAAACACCGTCTCGTGGGCTCGGAGATGTGTATAAGAGACAGGATAC | 31 |
| Trbv17 | TACGGTTAAGCAGAACCCGTCTCGTGGGCTCGGAGATGTGTATAAGAGACAGGGTGG | 32 |
| Trbv19 | CATCATTACTCAGACACCGTCTCGTGGGCTCGGAGATGTGTATAAGAGACAGGGAGC | 33 |
| Trbv20 | ACTCGTCTATCAATATCCGTCTCGTGGGCTCGGAGATGTGTATAAGAGACAGGACTC | 34 |
| Trbv21 | TGGGGTTGTCCAGAATCCGTCTCGTGGGCTCGGAGATGTGTATAAGAGACAGGATGC | 35 |
| Trbv23 | TGCAGTTACACAGAAGCCGTCTCGTGGGCTCGGAGATGTGTATAAGAGACAGGTTGC | 36 |
| Trbv24 | TGGAGTAACCCAGACTCCGTCTCGTGGGCTCGGAGATGTGTATAAGAGACAGAATTC | 37 |
| Trbv26 | AAAAGTCATTCAGACTCCGTCTCGTGGGCTCGGAGATGTGTATAAGAGACAGGACAT | 38 |
| Trbv29 | GAAAGTAACCCAGATGCCGTCTCGTGGGCTCGGAGATGTGTATAAGAGACAGAGTGT | 39 |
| Trbv30 | CCTCCTCTACCAAAAGCCGTCTCGTGGGCTCGGAGATGTGTATAAGAGACAGGCTCA | 40 |
| Trbv31 | GACTATCCATCAATGGCC | 41 |

A first PCR was performed in a tube with a Hotstart PCR mix for 50 cycles to enrich the TCR. Then a second PCR was performed using an Illumina P5 primer and to add the library index using a P7 primer. Briefly, 1 ng of cDNA was added with Qigen 1 × HotStar Taq buffer, 10 nM of mixed TCRα and TCRβ V segment primers, 1 µl of each dNTP, and 1 µl HotStar Taq and H₂O to make final volume 100 µl. The PCR cycle was as follows: 94° C. for 10 minutes followed by 50 cycles of 94° C. for 40 seconds, 62° C. for 45 seconds, 30 cycles of 94° C. for 40 seconds, 62° C. for 45 seconds, 72° C. for 1 minute, and a final incubation at 72° C. for 1 minute. The PCR products were cleaned up with Ampure bead and eluted to 25 µl. The second PCR was performed using 5x Kapa Mg²⁺ buffer, 1 µl DNTP, 1 µl KAPA HIFI enzyme, 0.2 µl IFC-F primer, 0.2 µl N7XX primer, H₂O to make final volume 50 µl in the following cycle:

| Kapa AMP | | |
|---|---|---|
| Step 1 | 72° C. | 3 minutes |
| Step 2 | 95° C. | 10 seconds |
| Step 3 | 95° C. | 30 seconds |
| Step 4 | 66° C. | 30 seconds |
| Step 5 | 72° C. | 1 minute |
| Step 6 | Go to step 3 | 14 times |
| Step 7 | 72° C. | 5 minutes |
| Step 8 | 4° C. | Forever |

The PCR products were again cleaned up using Ampure bead and eluted to 15 µl for Qubit quantification and size analyzed by bioanalyzer before sequencing on the Illumina Miseq (2 x 300 bp reads). The end result is a spatial single cell TCR-seq library that can (theoretically) map TCR clones back to regions in the tissue.

### Example 7: Use of XYZeq for Spatial ATAC-seq

The basic protocol is the same as XYZeq RNAseq protocol with reaction mix in the wells that will spatially barcode, then the entire chip is frozen to -80° C. so that the tissue can be place on top, after incubating for reaction, cells are taken out and then sorted into 96 well plates for second barcoding via PCR. Library is indexed and sequenced. An exemplary procedure is as follows:

1. Reaction mix consists of 5x DMF-TAPS buffer, 30 custom and uniquely indexed single sided Tn5 transposomes (10 ligated with barcoded P5 adaptor and 20 ligated with barcoded P7 adaptor), digitonin (tissue digestion reagent), and H20. By spotting TN5-P5 along the rows and Tn5-P7 along columns, it is possible to get 200 wells that will have unique barcoded Tn5 combinations.

2. The microwell array was sealed and incubated at 55° C. for 30 minutes and 37° C. for 15 minutes.

3. Following tagmentation, the microwell array was placed in a 50 ml conical tube with 40 mM EDTA (supplemented with 1 mM Spermidine, 20% FCS, and PBS) added to stop the reaction and vortexed. Cells in the conical tube were spun down, resuspended in 1 ml, filtered, and stained with DAPI. 25 DAPI+ cells were sorted into each well of 96-well plates that contained 12.5 µl lysis buffer (11 µl of EB buffer, 0.5 µl of 100X BSA, and 1 µl of DTT).

4. After sorting, indexed PCR primer to each well (0.5 µM final concentration), polymerase master mix was added to each well. Tagmented DNA is then PCR amplified.

5. After PCR amplification, DNA was cleaned up using 1X Ampure beads (Agencourt) and eluted in 15 µl of EB buffer, then quantified.

6. The concentration and quality of the libraries was determined using the BioAnalyzer.

### Example 8: XYZeq Reveals Expression Heterogeneity in the Tumor Microenvironment

Single-cell RNA-sequencing (scRNA-seq) of tissues has revealed remarkable heterogeneity of cell types and states but does not directly provide information on the spatial organization of cells within complex tissue architecture. To better understand how individual cells function within an anatomical space, we developed XYZeq, a novel workflow that encodes spatial metadata into scRNA-seq libraries. We used XYZeq to profile heterotopic mouse liver and spleen tumor models to capture transcriptomes from tens of thousands of cells across eight tissue slices. Analyses of these data revealed the spatial distribution of distinct cell types and a cell migration-associated transcriptomic program in tumor-associated mesenchymal stem cells (MSCs). Furthermore, we identify localized expression of tumor suppressor genes by MSCs that vary with respect to proximity to the tumor core. We demonstrate XYZeq can be used to simultaneously map the transcriptome and spatial localization of individual cells in situ to reveal how cell composition and cell states can be affected by location within complex pathological tissue.

### 1. Materials and Methods

### i. Mice, Tumor Cell Line, and Tumor Inoculation

6-12 weeks old C57BL/6 female mice were purchased from Jackson Laboratories and housed in specific pathogen free conditions. MC38 colon adenocarcinoma cell line was cultured in complete cell culture medium (RPMI 1640 with GlutaMAX, penicillin, streptomycin, sodium pyruvate, HEPES, NEAA, and 10% fetal bovine serum (FBS). Cell lines were routinely tested for mycoplasma contamination. For experiments, mice were given an anesthetic cocktail of Buprenorphine (300 ul) and Meloxiacam (300 ul) 30 minutes prior to the procedure. At the time of surgery, 1 drop of Bupivacaine was administered and mice were anesthetized with isoflurane prior to intrahepatic (or intrasplenic) injection of MC38 colon adenocarcinoma cells (50 µl at 10x10⁶ cells/ml) using a 30 ½ gauge needle. Incision was stapled closed and post-operative care was given to the mice. All experiments were conducted in accordance with the animal protocol approved by the University of California, San Francisco IACUC committee.

### ii. Cancer Model System

Intrahepatic and intrasplenic cancer model that we used for the paper is described in great detail in recently published report, Lee et al. 2020 (21). Briefly, intrahepatic and intrasplenic tumors were generated by subcapsular injection of the tumor cells directly into the organs. To establish the ideal time point for sacrificing the mice, in vivo imaging was done on tumor inoculated mice. Intra-organ injected MC38 cells were modified to express the firefly luciferase. Mice were intraperitoneally infected with D-luciferin (150 mg/kg; Gold Biotechnology) 7 minutes prior to imaging with the Xenogen IVIS Imaging system. Mice with detectable tumor nodules with at least 5 mm fluorescence were sacrificed for tissue harvesting. Organs to be used for XYZeq were fixed with dithiobis(succinimidyl propionate) (DSP) (Thermo Scientific) and cryopreserved while organs used for 10X Genomics Chromium Single cell sequencing were digested in RPMI complete medium that were supplemented with collagenase D (125 U/ml; Roche) and deoxyribonuclease I (20 mg/ml; Roche) then processed for single cell suspension using the gentleMACS tissue dissociator per manufacturer’s protocol (Miltenyi).

### iii. 10X Genomic Chromium Platform

Cells isolated from tissue were washed and resuspended in PBS with 0.04% BSA at 1000 cells/µl and loaded on the 10X Genomics Chromium platform per manufacturer’s instructions and sequenced on NovaSeq or HiSeq 4000 (Illumina).

### iv. Tissue Harvesting and Cryopreservation

At day 10 post tumor inoculation, mice were sacrificed and harvested for the tumor injected liver (or spleen) and incubated for 30 minutes in ice cold DMSO-free freezing media (Bulldog Bio). This was followed by 30 minutes incubation in ice cold DSP (Thermo Scientific) supplemented with 10% FCS, then neutralized in ice cold 20 mM of Tris-HCl, pH 7.5. The organs were placed in a cryomold, sealed airtight, and slowly frozen overnight in -80° C.

### v. Cells and Reagent Dispensing Into Array

The sciFLEXARRAYER S3 (Scienion AG) was used to dispense cells and reagents to the microwell arrays. Drop stability and array quality were assessed for each experiment. Prior to dispensing into the microwell arrays slides, Autodrop detection was used to assess drop stability and quantify the velocity, deviations, and drop volume for each reagent. Volume entry was used to determine the number of drops required to reach the total designated well volume. Each well oligo(dT) primer 5′ CTACACGACGCTCTTCCGATCTNNNNNNNNNN[16bp unique spatial barcode] TTTTTTTTTTTTTTTTTT-3′, where “N” is any base; SEQ ID NO: 43; IDT) were spotted. During barcoding, the dewpoint control software monitored the ambient temperature and humidity allowing dynamic control of the temperature of the source plate to maintain nominal oligo concentrations through the duration of the run. Barcoded slides were dried in the wells prior to storage. Reaction mix (Thermo Fisher Scientific) were added to wells and automated with a 10% bleach wash between each probe to eliminate carry over contamination. Dissociation/permeabilization buffer was printed into each well on day of experiment and tissue section was loaded onto the microwell array slides. For all tissue experiments, DSP fixed HEK293T cells were added at 5 µl (@ 10x10⁶ cells/ml) to the RT digestion mix before being dispensed across all the wells in the microarray. The average number of HEK293T cells were 58 cells/well, however, the absolute number of cells per well likely varied across the array due to the cells being in suspension inside the dispensing nozzle. Cells harvested from the array after incubation was analyzed on ARIA (BD biosciences) and datasets were analyzed using FlowJo software (Tree Star Inc.).

### vi. Array Fabrication

Photoresist masters are created by spinning on a layer of photoresist SU-8 2150 (Fisher Scientific) onto a 3-inch silicon wafer (University Wafer) at 1500 rpm, then soft baking at 95° C. for 2 hours. Then photoresist-layered silicon wafer is exposed to ultraviolet light (UV) for 30 minutes over a photolithography masks (CAD/Art Sciences, USA) that was printed at 12,000 DPI. After ultraviolet exposure, the wafers are hard baked at 95° C. for 20 minutes then developed for 2 hours in fresh solution of propylene glycol monomethyl ether acetate (Sigma Aldrich) to develop, followed by a manual rinse with fresh propylene glycol monomethyl ether acetate then baked at 95° C. for 2 minutes to remove residual solvent. Polymethylsiloxane (PDMS) mixture (Sylgard 184, Dow Corning Midland) with pre-polymer:curing-agent ratios of 10:1 was poured over the SU-8 silicon wafer master. This was placed in a 100 mm petri dish and was cured overnight in a 70° C. oven. This PDMS negative mold was peeled off the SU-8 silicon master the following day. PDMS block was placed on a flat surface and Norland Optical Adhesive 81(NOA81) (Thorlabs) was poured into the mold to cover the entire surface. A slide was placed on top of the NOA-poured PDMS mold, and a transparent weight was placed on top. NOA was cured for 2 minutes under UV light, flipping once half way thru the UV curing time. Finally, PDMS mold was detached from the cured NOA microwell array slide (referred to as microwell array chips). The dimensions of each hexagonal well is approximately 400 µm in height and 500 µm in diameter with the volume of 0.04 mm³ which can hold 40 nl of liquid.

### vii. XYZeq Methodology

Liver/tumor organ was mounted on a Cyrostat (Leica) and sliced at 25 µm for use as a XYZeq experimental sample or mounted on a histology slide at 10 µm for immunohistochemical staining. On the day of experiment, XYZeq microwell array chips were spotted with reverse transcription cocktail mix that were spiked in with fixed HEK293T cells. The microwell array chips were brought down to -80° C. and tissue slice was placed on top of the array. A digital image was taken to document the orientation of the tissue before sandwiching a silicone gasket sheet between the XYZeq microwell array chip and a blank histology slide. The chip was placed in a Microarray Hybridization Chamber (Agilent) to ensure an air tight seal while undergoing tissue digestion and reverse transcription. In order to recover high quality RNA from fixed frozen tissue, the Microarray Hybridization Chamber housing the chip had to undergo a gradual step-wise temperature increase to 42° C. before the 20 minutes incubation to undergo reverse transcription. The chip was removed from the chamber and placed in a 50 ml conical tube with 50 ml of 1x SSC buffer and 25% FCS. The tube was vortexed and spun down at 1000 rcf for 10 minutes. Excess volume was removed and cells were filtered and stained for DAPI (Life Technologies) prior to sorting (BD Aria) into 96 well plates preloaded with 5 µl of second RT mix. Plates were reverse transcribed for 1.5 hours at 42° C., followed by PCR using 2x Kapa Hotstart Readymix (Kapa Biosystems). PCR amplification was performed with indexing primer (5′-AATGATACGGCGACCACCGAGATCTACAC[i5]ACACTCTTTCCCTACACGACGCTCTT CCGATCT-3′; SEQ ID NO: 44; IDT). Contents of the PCR plate were pooled into 2 ml Eppendorf tubes and cDNA was purified with AMpure XP SPRIbead (Beckman). cDNA was tagmented and amplified with Illumina Nextera library p7 index (IDT). Final library was analyzed by BioAnalyzer (Agilent) and quantified by Qubit (Invitrogen) and sequenced on a NovaSeq or HiSeq 4000 (Illumina) (read 1:26 cycles, read 2: 98 cycles, index 1: 8 cycles, index 2: 8 cycles).

### viii. XYZeq Decontamination Analysis

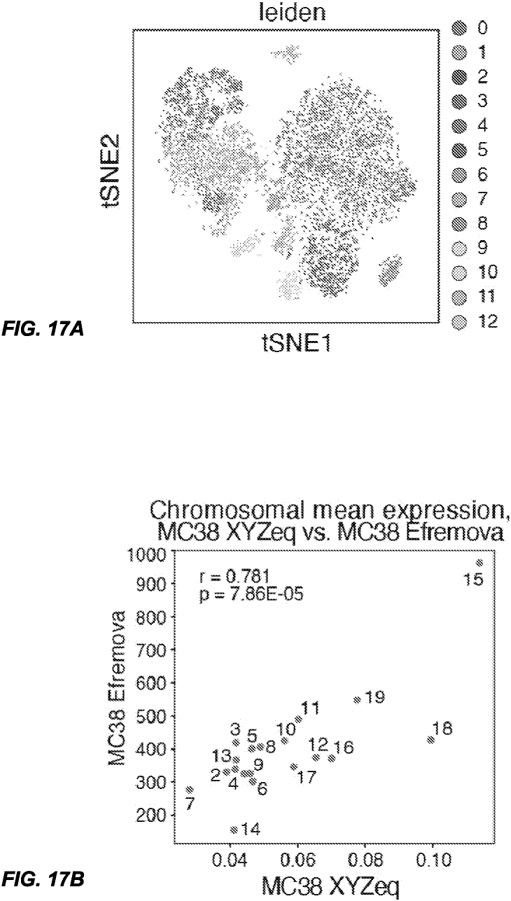

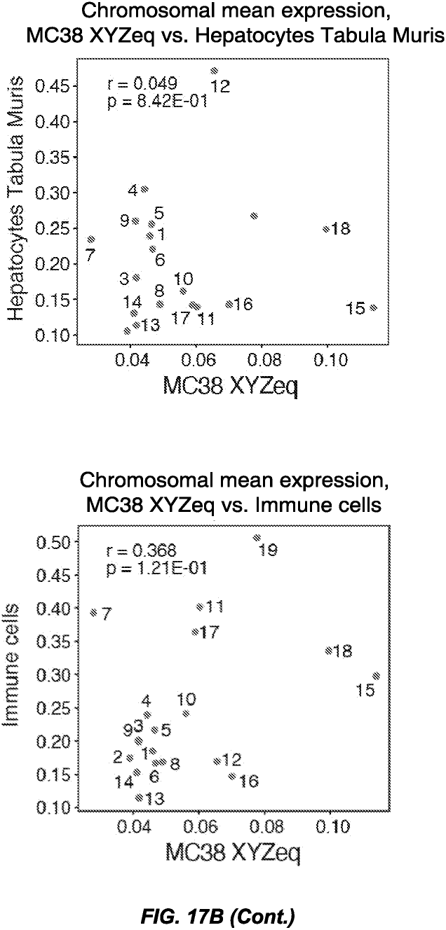

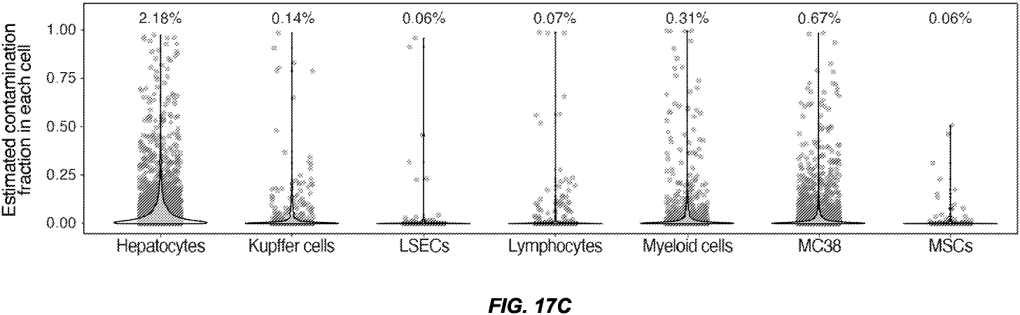

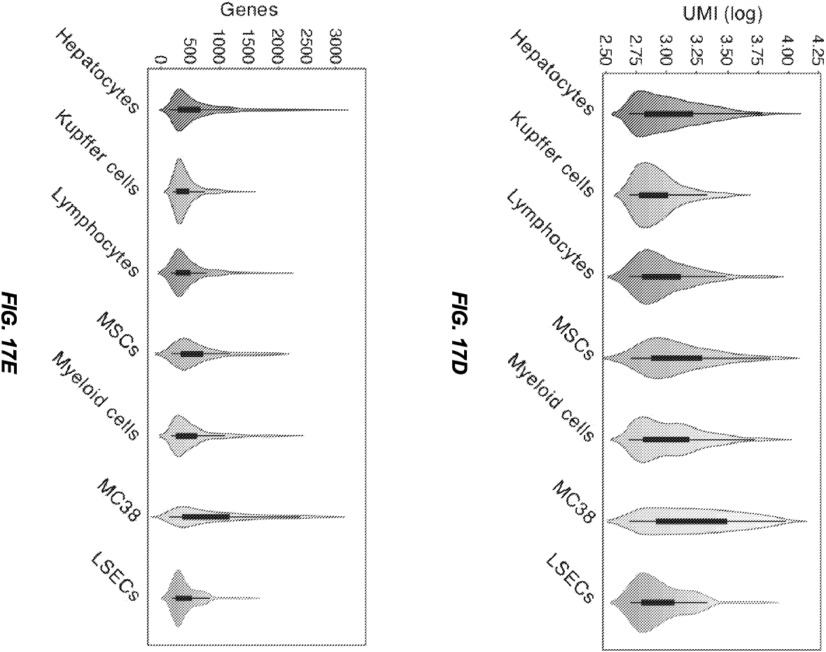

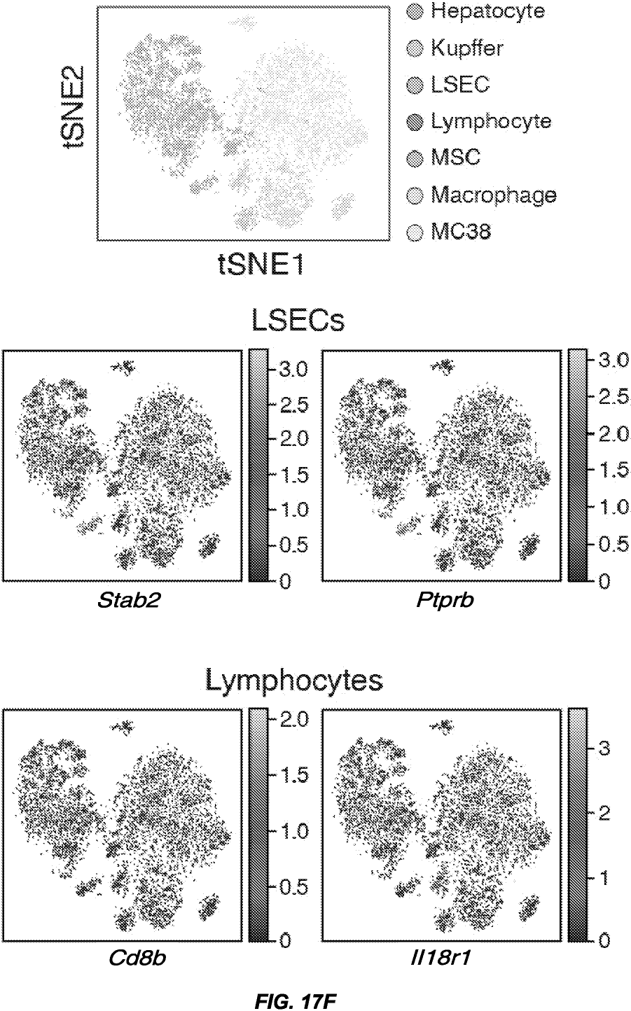

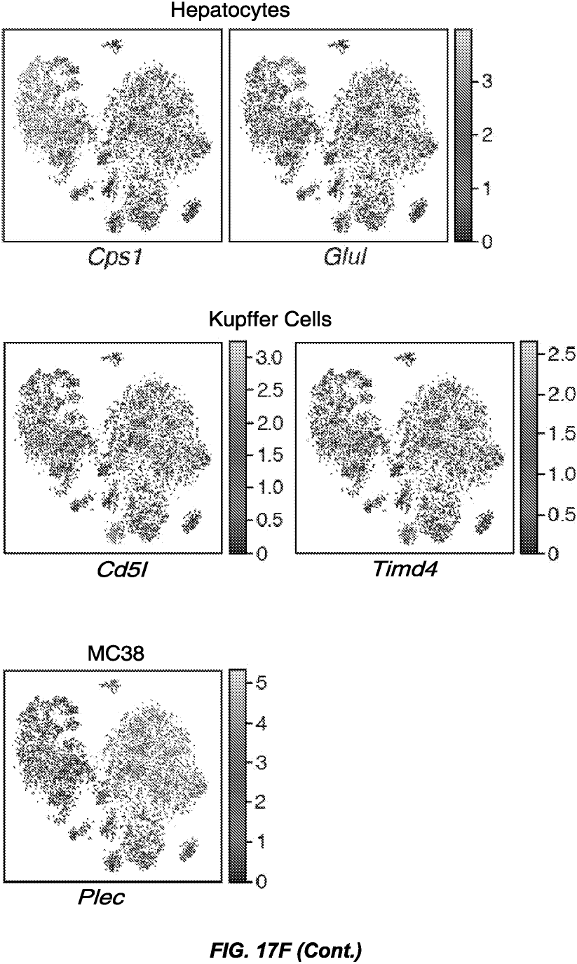

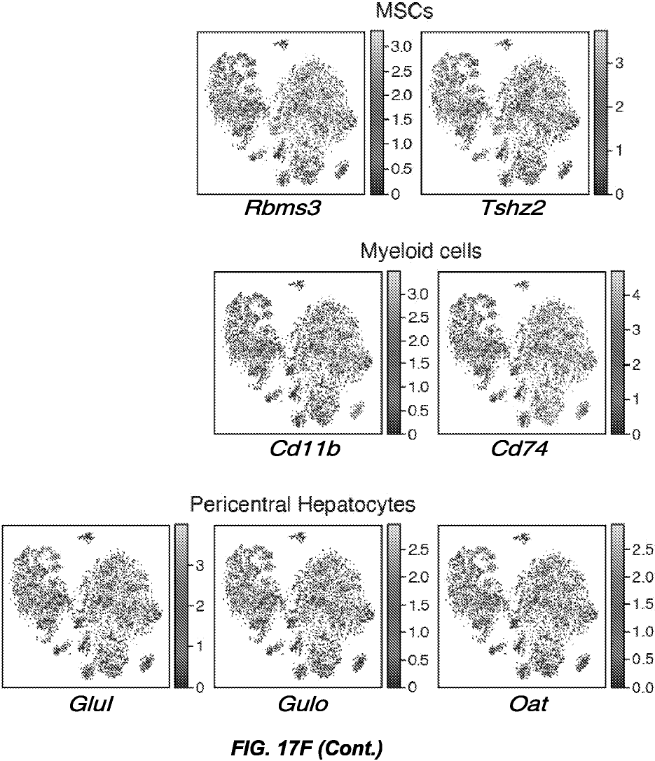

In our analysis, we recognized some reads aligning to the mouse genes were present in cells that otherwise had high alignment to the human genome. We suspected these reads were ambient RNA contamination, and sought to remove them. We first removed mouse-aligned transcripts with an extremely high expression in human cell population (n = 59, log(counts + 1) > 6). The human cell population was considered a control in the contamination detection, because any ambient RNA from lysed cells was expected to contaminate both mouse and human cells. DecontX (2) was then performed to estimate the contamination rate for different cell populations using the human-mouse mixture dataset, and therefore derive a decontaminated count matrix from the raw data. Briefly, the algorithm applies variational inference to model the observed counts of each cell as a mixture of true gene expression of its corresponding cell population and the contamination signature (from other cell populations), and then subtracts the contamination signature (FIG. 17C). By considering the human-mouse mixed species experiment, we could remove those counts potentially contributing to collision, and effectively account for all potential transcripts in the lysed cells which contribute to ambient RNA. In FIG. 17C, the initial estimated contamination rate for each mouse cell type are plotted with the median estimates ranging from 0.06% - 0.31% with the highest seen in the hepatocyte cell cluster with 2.18% initial contamination fraction. All the downstream analysis was performed based on the decontaminated data after contamination removal.

### ix. How Distinctions Were Made Between Collision Rate and Contamination Rate

The collision rate is directly calculated from the gene expression of human-mouse mixture dataset based on the ratio between mouse-aligned and human-aligned transcripts, while the contamination rate for each cell is estimated as a cell-specific parameter in the Bayesian hierarchical model via variational inference from DecontX. In order to specify the contamination rate, each cell has a beta-distributed parameter modeling its proportion of transcript counts which come from its native expression distribution. The estimated contamination rate for each cell is the proportion of transcript counts which come from contamination in the Bayesian model. Each transcript in a cell follows a multinomial distribution parameterized by the native expression distribution of its cell population or contamination from all the other cell populations, given a Bernoulli hidden state, indicating whether the transcript comes from its native expression distribution or from the contamination distribution.

### x. Cell Species Mixing Experiment

Mixture of HEK293T and NIH/3T3 cells were deposited into wells in a gradient pattern across the columns of the array with a total of 11 distinctive cell proportion ratios. Specifically columns on the array was spotted with human cells to mouse cells ratio of 100/0; 90/10; 80/20; 70/30; 60/40; 50/50; 40/60; 30/70; 20/80; 10/90; 0/100; 10/90; 20/80; 30/70; 40/60; 50/50; 60/40; 70/30; 80/20; 90/10; 100/0, with only human cells flanking the end columns and only mouse cells in the center columns. The ratio of UMI de-duplicated reads aligning to either human or mouse reference genomes were calculated for each cell, and those with less than 66% aligning to a single species were deemed barcode collision cells.

### xi. XYZeq Single Cell Analysis

Single cell RNA sequence data processing was performed where sequencing reads were processed as previously described (17). Briefly, raw base calls were converted to FASTQ files and demultiplexed on the second combinatorial index using bcl2fastq v2.20. Reads were trimmed using trim galore v0.6.5, aligned to a mixed human (GRCh38) mouse (mm10) reference genome and UMI deduplicated. Reads were then assigned to single cells by demultiplexing on the first combinatorial index, prior to the construction of a gene by cell count matrix. The count matrix was processed using the Scanpy toolkit. Cells with less than 500 UMIs and greater than 10000 UMIs, as well as cells expressing less than 100 unique genes or more than 15000, were discarded. Cells with more than 1% mitochondrial read percentage were also discarded. Gene counts were normalized to 10,000 per cell, log transformed, and further filtered for high mean expression and high dispersion using the filter genes dispersion function, with a minimum mean of 0.35, maximum mean of 7, and minimum dispersion of 1. Gene counts were then corrected using the regress out function with total counts per cell and the percentage mitochondrial UMIs per cell as covariates. Subsequent dimensionality reduction was done by scaling the gene counts to a mean of 0 and unit variance, followed by principal component analysis, computing of a neighborhood graph, and t-distributed stochastic neighbor embedding (tSNE). Leiden clustering was performed with a resolution of 0.8, and cells were grouped to reveal distinct murine cell types and human HEK293T cells.

### xii. 10X Data Processing

Counts matrices were generated using the “count” tool from Cellranger version 3.1.0, using the combined human and mouse reference dataset (version 3.1.0) and the “chemistry” flag set to “fiveprime.” The count matrix was processed using the Scanpy toolkit. Cells with less than 500 UMIs and greater than 75,000 UMIs, as well as cell expressing less than 100 unique genes and greater than 10,000, were discarded. Cells with more than 7.5% mitochondrial read percentage were also discarded. Gene counts were normalized to 10,000 per cell, log transformed, and further filtered for high mean expression and high dispersion using the filter genes dispersion function, with a minimum mean of 0.2, maximum mean of 7, and minimum dispersion of 1. Gene counts were then corrected using the regress out function with total counts per cell and the percentage mitochondrial UMIs per cell as covariates. Subsequent dimensionality reduction was done by scaling the gene counts to a mean of 0 and unit variance, followed by principal component analysis, computing of a neighborhood graph, and tSNE. Leiden clustering was performed with a resolution of 1, and cells were grouped to reveal major murine cell types and human HEK293T cells.

### xiii. Heatmap for XYZeq

Mouse cells were subsetted from the XYZeq processed data matrix. The processed gene expression values were plotted in a heatmap with a minimum fold change of 1.5 and hierarchically clustered using the heatmap function from Scanpy, with the default settings of Pearson correlation method and complete linkage.

### xiv. XYZeq Gene Pairplot

Four slices of liver/tumor tissue were processed using the XYZeq assay (with HEK293T cells spiked-in) and aligned to a joint human and mouse reference. All genes with at least one count in each slice were kept, and the counts across the common set of genes between pairwise slices were plotted in the lower triangle, with the Spearman correlation for the data shown in the upper triangle. Along the diagonal, histograms were plotted showing the distribution of counts per gene for all the non-zero genes for each slice.

### xv. XYZeq Cell/Well Pairplot

Pairplot showing the number of microwells containing pairwise combinations of cell types. For scatter plots, each point in the plot represents a well, and its coordinate positions indicates the number of cells of each cell type present in that well. Every dot on the scatter plot is a gene representing mean per gene for common genes across all cells in the slices. Along the diagonal of the figure are histograms, showing the univariate distribution of cell number per well for the given cell type.

### xvi. Heat Map Comparing 10X to XYZeq

Mouse cells were subsetted from each of the processed data matrices. For pairwise mouse Leiden clusters found between XYZeq and 10X, the scaled and log transformed gene expression values of common genes were plotted. For each comparison, a Pearson correlation was calculated and plotted in the heatmap. Row/column labels were ordered according to their corresponding cell types.

### xvii. Correlation Plot

Mouse cells were subsetted from each of the processed data matrices. Proportions for each cell type (as determined by the Leiden clustering and visualized using tSNE) were plotted, and the coefficient of determination was calculated by fitting to the model that assumes proportions are equal between the two assays.

### xviii. Gene Module Analysis of Top Contributing Genes

In order to identify gene modules using non-negative matrix factorization genes expressed in fewer than 5 cells, and cells expressing fewer than 100 genes were filtered out. Variance stabilizing transformation was performed on count data, and confounding covariates including number of counts per cell, batch, and mitochondrial read percentage were regressed out by a regularized negative binomial regression model using the SCTransform (48) function in the Seurat R package. Pearson residual values from the regression model were centered, and all negative values were converted to zero. Non-smooth non-negative matrix factorization (nsNMF) was performed on the resulting expression data with a rank value of 20 using the nmf (49) function in NMF R package. In each module, genes were sorted by their magnitude in the corresponding coefficient matrix in a descending order. Gene ontology enrichment analysis was performed for the sorted genes in each module using GOrilla (50). For each module, the top consecutive genes with higher coefficients in this module compared to all the other modules were further selected as genes contributing the most to the module (51) in the tissue-specific analysis. Binary spatial plots were generated by first calculating the median expression across all the cells for each well within each batch based on the log-normalized gene expression data. We then extracted the mean expression across all the genes within one module for each well and calculated the average of mean expression across selected module genes for each well weighted by the number of cells in each well. The wells with a mean expression across genes above the weighted average were labeled as highly expressing for that gene module, and all the other wells with non-zero expression of those selected module genes were labeled as lowly expressing that gene module. tSNE plots representing the gene modules were colored by their mean expression of genes within the annotated module.

### xix. Overlapping Analysis Between the Gene Modules Identified in Liver/Tumor and Spleen/Tumor

Gene modules were first identified using nsNMF with a rank value of 20 for the two tissues, liver/tumor and spleen/tumor, respectively. The top 200 genes in each sorted gene list for a module were selected as having high association with the module. For each module in the liver/tumor tissue, the spleen/tumor module with the largest gene overlap was initially matched as functionally similar. We then removed those matched pairs with fewer than 25% overlapping genes out of top 200 genes in the liver/tumor module. In order to calculate cell type fractions that make up each module, the average gene expression for each gene across all the cells was calculated. Median expression across all the overlapping genes for each cell type was further computed, which was later transformed into fractions by dividing by the sum of median expression across all the cell types.

### xx. Defining the Proximity Score by Wells

We sought to define a score for each well of the hexagonal well array that would capture how centrally located a well was within either the tumor or non-tumor tissue domains. Central to the method was the determination of successive concentric “layers” of wells that were adjacent to a well in question: those corresponding to its immediate neighbors (layer 1), those wells exactly 2 wells away (layer 2), and so on, for n layers. In the spleen/tumor, we selected several wells on the far side of the tumor region and set the score of these wells to 1. We then took 10 successive layers of wells and decreased the score linearly with each layer, with the wells in layers 10 and beyond set to 0. In the liver, MC38 cells were found in different locations, and therefore, unlike the spleen, there was no single unidirectional spatial dimension to place all MC38 cells at one end and all non-tumor tissue cells at the other. Therefore, we used an alternative approach to calculate these scores in the liver/tumor tissue. For each well W_{x,y}, annotated by their x, y position on the hexagonal well array, we calculated the proportion of hepatocytes, P_{x,y}, since the hepatocytes were the most abundant parenchymal cell type in, and strictly associated with, the non-tumor liver tissue:

$${t}_{x , y} = \# o f t o t a l h e p a t o c y t e s a n d M C 38 c e l l s i n {w}_{x , y}$$

$${h}_{x , y} = \# o f h e p a t o c y t e s i n {w}_{x , y}$$

$${p}_{x , y} = \frac{{h}_{x , y}}{{t}_{x , y}}$$

Then, for each well in question W_{x,y}, we tabulated the surrounding wells in each of the successive concentric 10 layers. We denote these wells w_{x′y′} to differentiate from the well in question. For each of those layers l, we took its constituent wells’ p_{x′,y′} and calculated a cell number-weighted average P_{x,y,l}:

$${w}_{x , y , l} = \left\{{w}_{{x}^{′} {y}^{′}} ∈ l a y e r l o f {w}_{x , y}\right\}$$

$${t}_{x , y , l} = \# o f t o t a l h e p a t o c y t e s a n d M C 38 c e l l s i n {w}_{x , y , l}$$

$${p}_{x , y , l} = {\sum }_{{x}^{′} , {y}^{′}}^{{w}_{x , y , l}} \frac{{t}_{{x}^{′} , {y}^{′}}}{{t}_{x , y , l}} {p}_{{x}^{′} , {y}^{′}}$$

Then, for the well in question W_{x,y}, we calculated a distance weighted average of all the p_{x,y,l,} and this became the proximity score s_{x,y} for the well in question. The distance weights for each layer, uₗ, were based on an exponential decay, terminated to 10 terms and then normalized to 1 by dividing by the sum of all weights uₛ. We give equal weight to p_{x,y} and the value for the layer 1 neighbors p_{x,y,1}. A decay factor d of 1.05 was chosen empirically, as it seemed to create the most uniform-like distribution of the scores across all wells.

$$d = 1.05 ,$$

$${u}_{s} = {\sum }_{l = 1}^{10} \frac{1}{{d}^{l}}$$

$${u}_{l} = \frac{\left(\frac{1}{{d}^{1}}\right)}{{u}_{s}}$$

$${s}_{x , y} = {u}_{1} {p}_{x , y} + {\sum }_{l = 1}^{10} {u}_{l} {p}_{x , y , l}$$

These calculations were repeated for all wells containing at least 1 murine cell.

### xxi. Trajectory Inference Analysis

Genes expressed in fewer than 5 cells, and cells expressing fewer than 100 genes were excluded. Variance stabilizing transformation was performed using the SCTransform (48) function in the R Seurat package. The resulting corrected count data in MSC in one tissue was used as the count matrix input in trajectory inference analysis, using the tradeSeq (41) package in R. Genes whose expression is associated with the proximity score were identified by the associationTest function in tradeSeq, based on a Wald test under the negative binomial generalized additive model. The p-values were corrected using Benjamimi-Hochberg multiple testing procedure, and genes with corrected p-values smaller than 0.05 were considered to be significantly associated with the proximity score.

### 2. Results

We have developed XYZeq, a method that uses two rounds of split-pool indexing to encode the spatial location of each cell from a tissue sample into combinatorially-indexed scRNA-seq libraries (17, 18). Critical for the performance of XYZeq, we fixed tissue slices with dithio-bis(succinimidyl propionate) (DSP), a reversible cross-linking fixative that has been shown to preserve histological tissue morphology while maintaining RNA integrity for single cell transcriptomics (19). In the first round of indexing, a fixed and cryo-preserved tissue section is placed on and sealed into an array of microwells spaced 500 µm center-to-center. The microwells contain distinctly barcoded reverse transcription (RT) primers (spatial barcode). This step physically partitions intact cells from tissue into distinct in situ barcoding reactions. After reverse transcription, intact cells are removed from the array, pooled, and distributed into wells for a second round of PCR indexing, imparting each single cell with a combinatorial barcode (FIG. 5A and FIG. 5B). After sequencing and demultiplexing, the spatial barcode maps each cell back to its physical location in the array (FIG. 5B). This combinatorial barcoding strategy theoretically could enable spatial transcriptomic analysis of large sets of single cells - with two rounds of split-pool indexing, 768 spatial RT-barcodes and 384 PCR-barcodes, up to 294,912 unique single-cell barcodes can be generated.

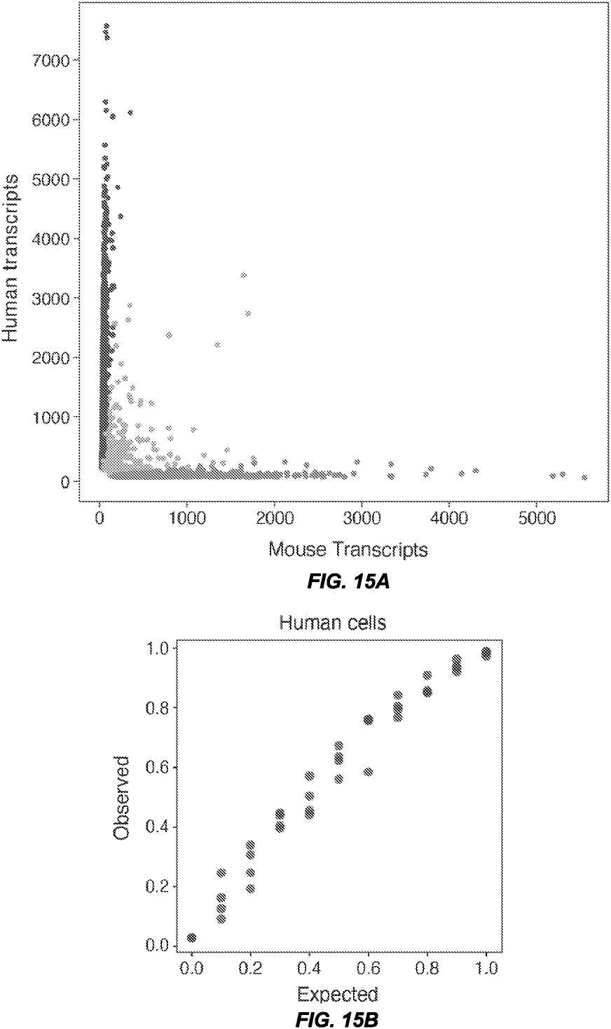

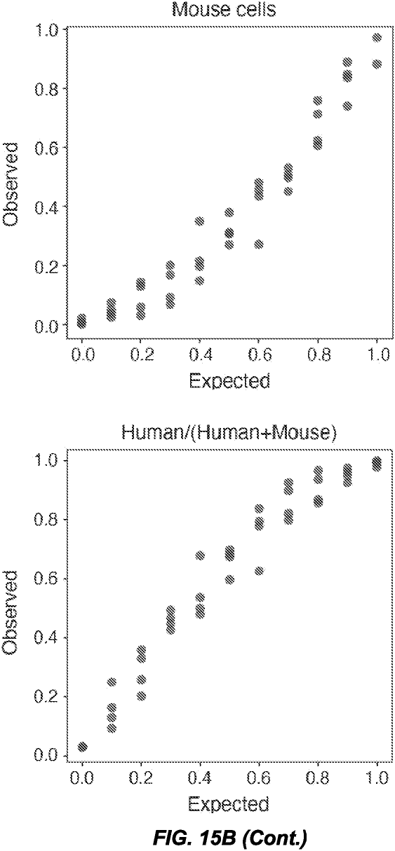

In order to determine whether XYZeq can assign transcriptomes to single cells, we performed a mixed species experiment where a total of 11 distinct ratios of DSP-fixed human (HEK293T) and mouse (NIH/3T3) cell mixtures were deposited into each of the 768 barcoded microwells, creating a cell proportion gradient along the columns of the array (FIG. 5C and Methods). XYZeq was used to generate scRNA-seq data for 6,447 cells. 94.8% of cell barcodes were assigned to a single species with an estimated barcode collision rate of 5.1% based on the percentage of cell barcodes with reads mapping to both human and mouse transcriptomes (FIG. 15A). We hypothesized that a portion of collisions were due to contamination from ambient RNA released by damaged cells. Using DeconX (20), a hierarchical Bayesian method that assumes the observed transcript counts of a cell is a mixture of counts from two binomial distributions, we removed contaminating transcripts, reducing the collision rate to 0.7% (FIG. 5D and Methods). After computational decontamination and removal of collision events, we obtained a median of 939 UMIs and 439 genes per human cell and 816 UMIs and 336 genes per mouse cell. Mapping each single cell to its originating microwell, we observed a high concordance between the observed and expected cell type proportions along the columns of the wells (Lin’s Concordance Correlation Coefficient = 0.91; FIG. 5E and FIG. 15B). Together, these results demonstrate that a minimal amount of barcode contamination takes place from single cells in each well and between neighboring wells on the array after pooling, indicating that the XYZeq workflow successfully produces spatially resolved scRNA-seq libraries.

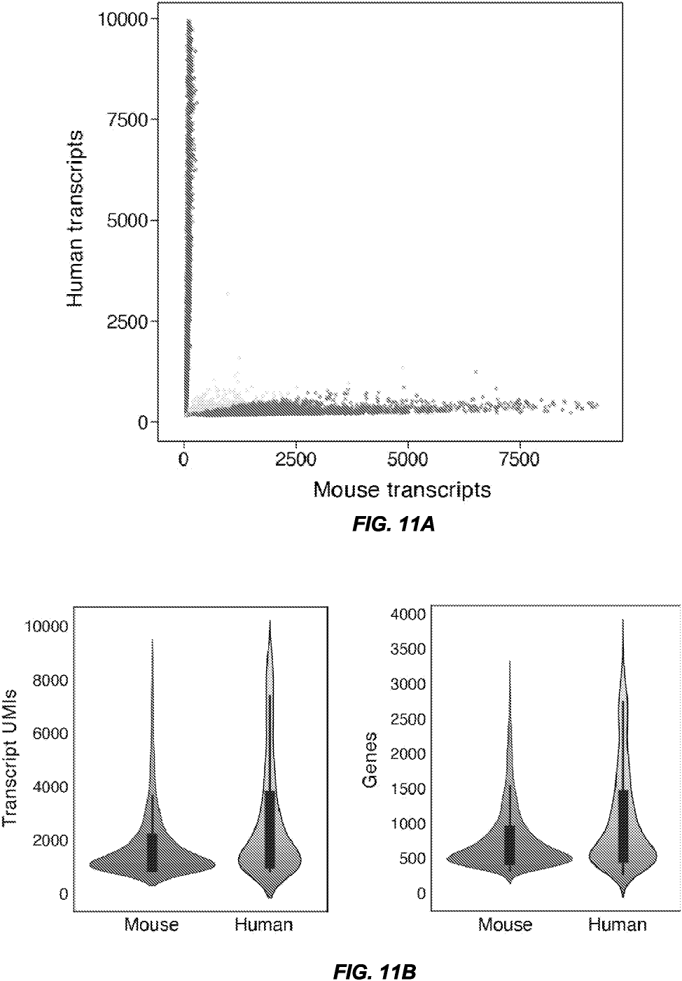

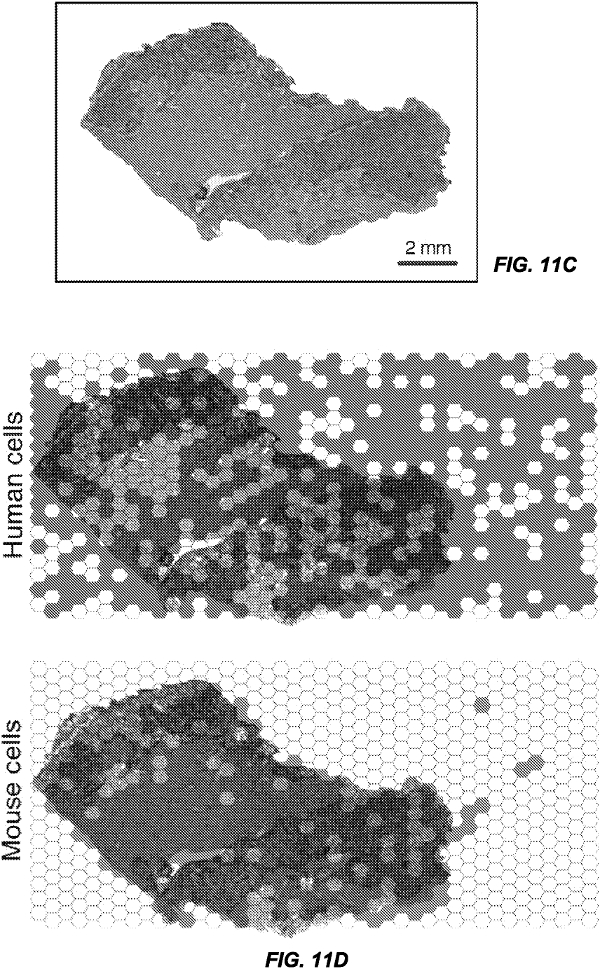

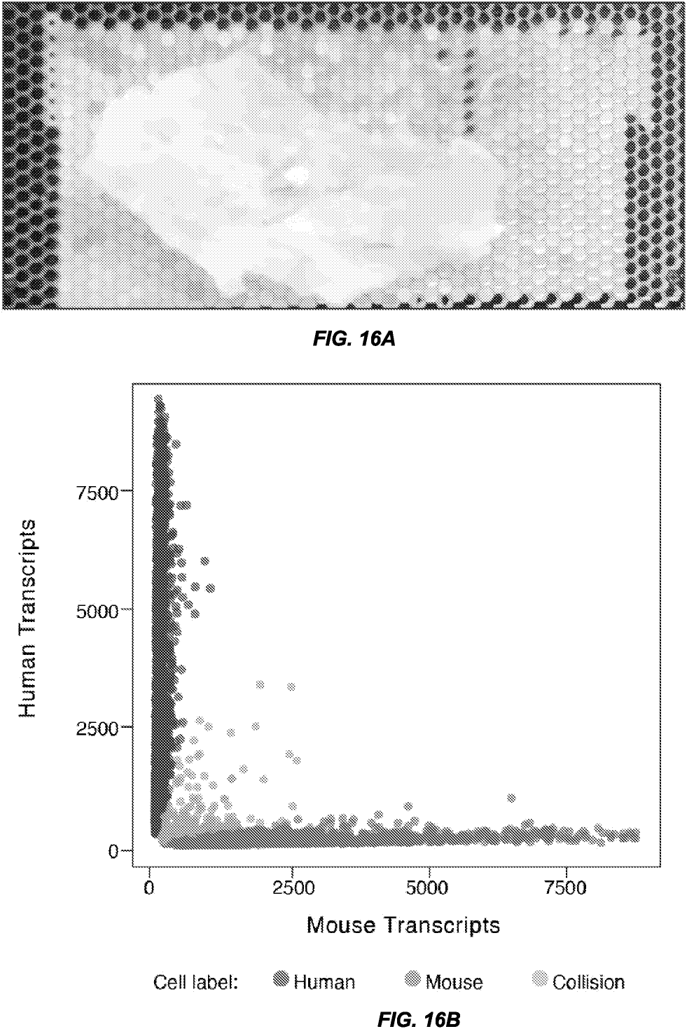

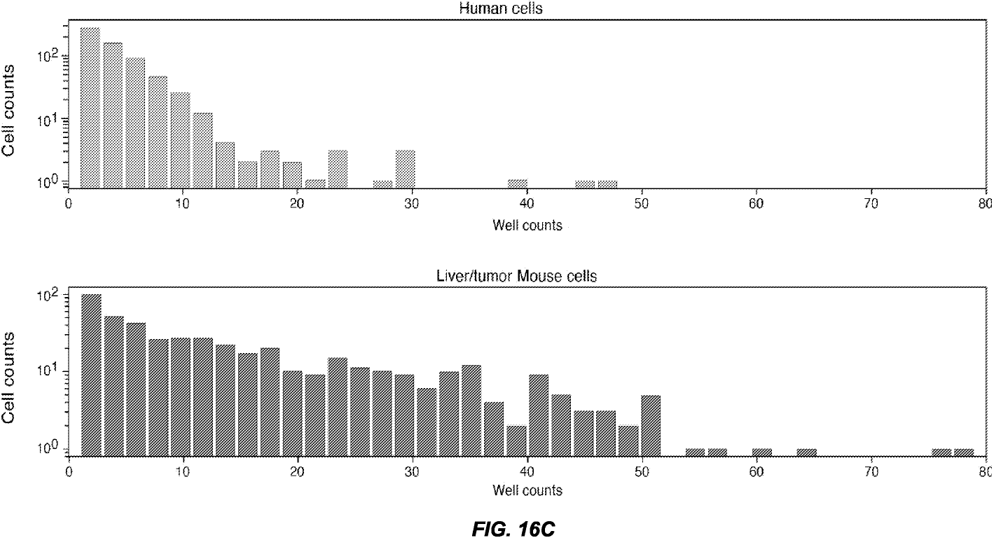

We next applied XYZeq to a fixed and cryopreserved heterotopic murine tumor model established by intrahepatic injections of a syngeneic colon adenocarcinoma cell line, MC38, into immunocompetent mice. This model mimics tissue infiltrating features of metastatic cancer, and more importantly, is associated with a relatively well-defined tumor boundary (21, 22). MC38 tumor cells also have immunomodulating properties with previous data showing immune cells infiltrating the tumor/tissue interface approximately 10 days post tumor inoculation (23, 24). Thus, we predicted that XYZeq could simultaneously capture the gene expression states and spatial organization of parenchymal liver cells, cancer cells, and tumor-associated immune cell populations. A 25 µm slice of fixed frozen liver/tumor tissue from a C57BL/6 mouse was placed on top of the pre-frozen microwell array while a sequential 10 µm slice was fixed for immunohistochemical staining (FIG. 16A and Methods). We also deposited fixed human HEK293T cells into the same array at an average of 58 cells per well to serve as a mixed-species internal control to experimentally quantify collision rates. We performed XYZeq and observed an initial collision rate of 7.3% based on comparing the ratio of human versus mouse transcripts (FIG. 16B). After computational decontamination and further quality control, which includes filtering cells based on cell counts and mitochondrial expression, the collision rate was reduced to 4.4% (FIG. 11A and Methods). After removing collisions, we obtained a total of 8,746 cells and detected a median of 1,596 UMIs and 629 unique genes per HEK293T cell and 1,009 UMIs and 456 unique genes per cell from the heterotopic murine tumor model at 46% sequencing saturation (FIG. 11B). A hematoxylin and eosin (H&E) stained serial section of the tissue revealed a histological boundary between the tumor and adj acent liver/tumor tissue (FIG. 11C). As expected, we observed HEK293T human cells distributed across the entire array, while mouse cells were sequestered within the boundary of the murine tissue (FIG. 11D). Note, empty spatial wells with no cells detected were likely due to a limited number of cells targeted for sequencing (~10,000). We obtained a median of 3 human cells/well and 9 mouse cells/well with a total of 13 cells/well expected (FIG. 16C).

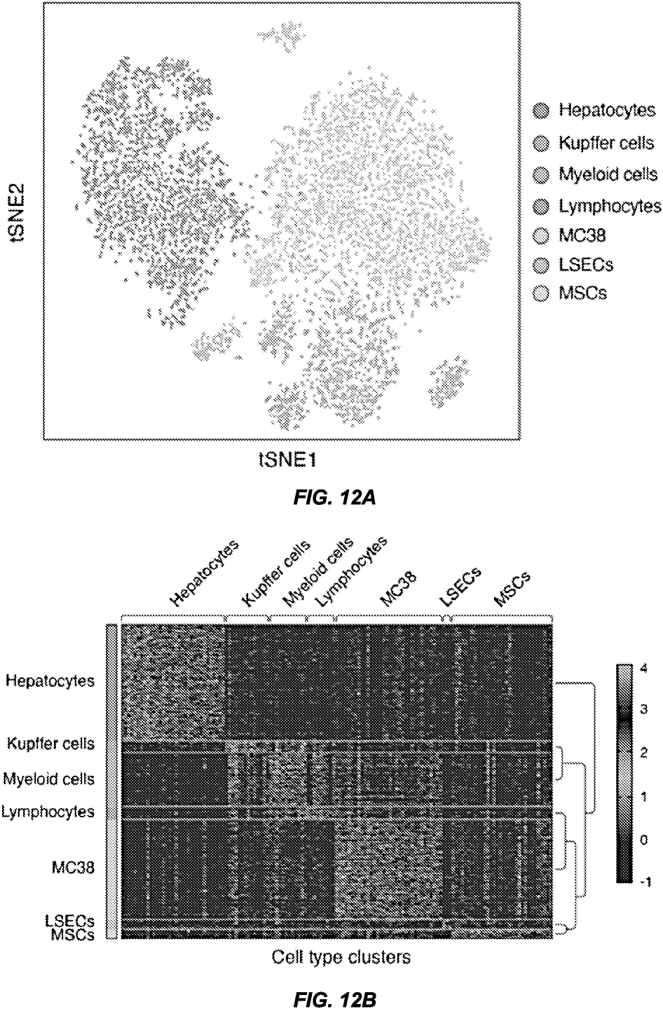

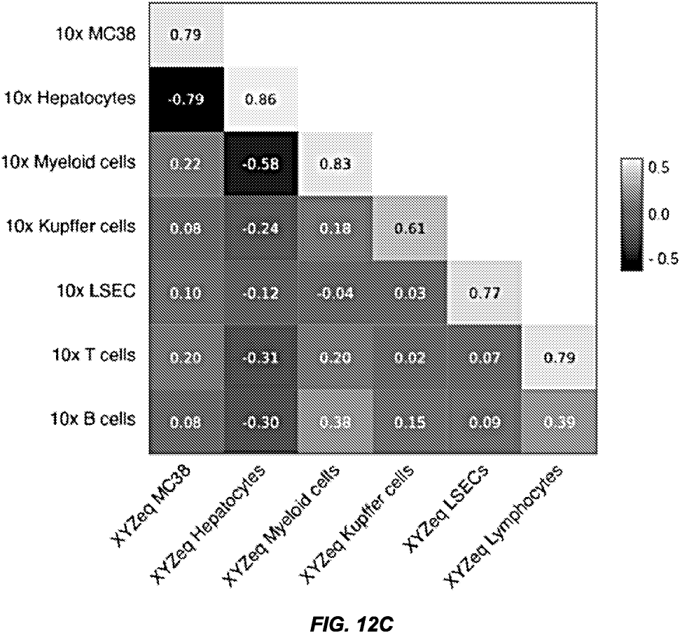

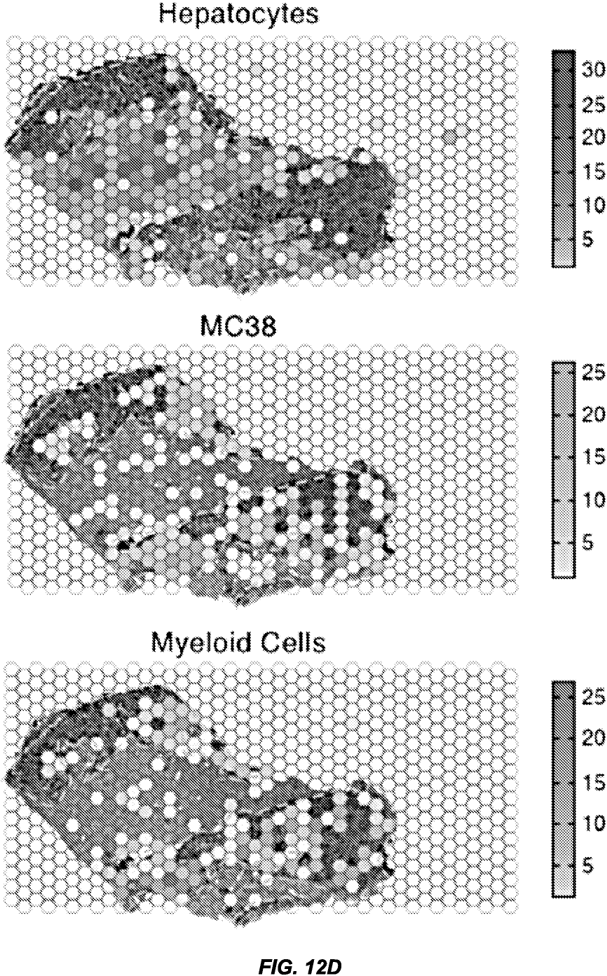

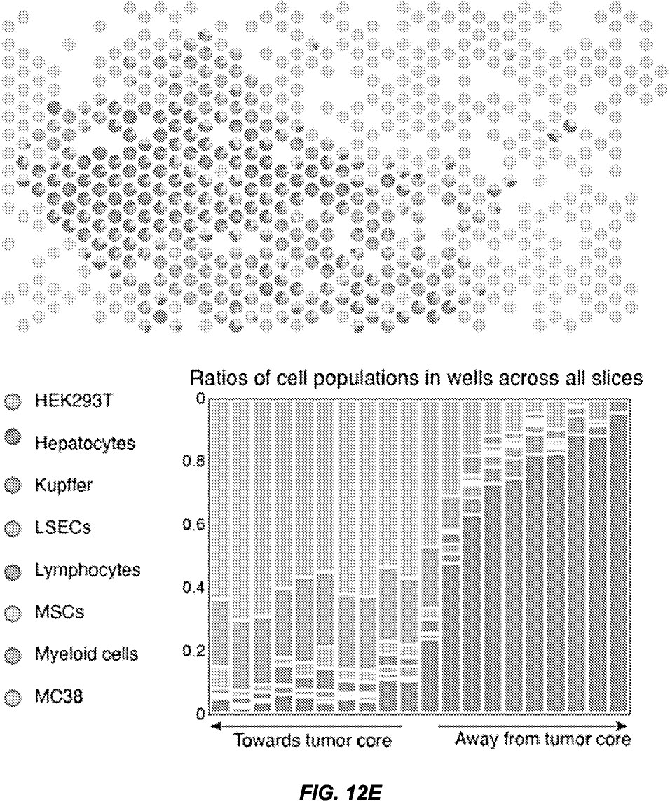

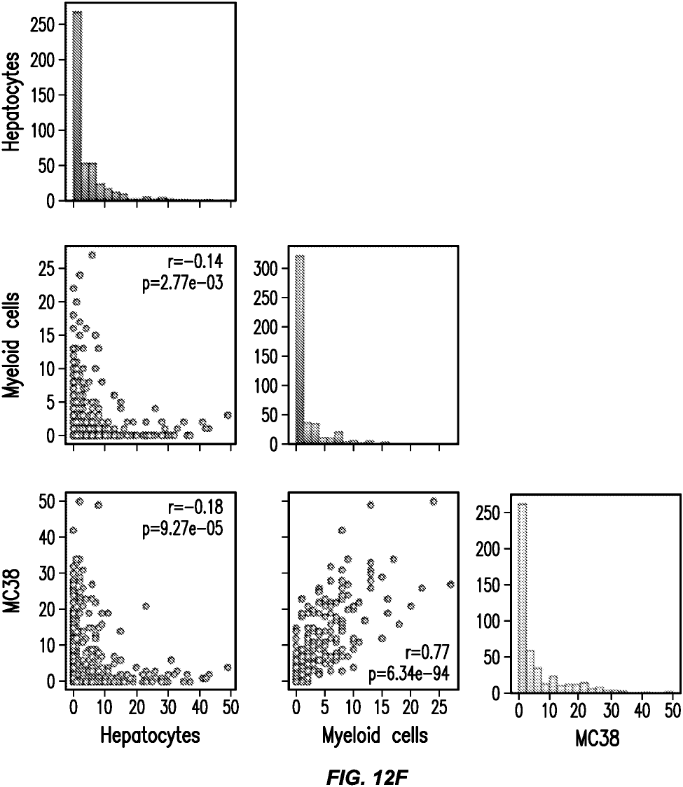

XYZeq revealed distinct cell types within the murine liver and tumor. Semi-supervised Leiden clustering revealed thirteen cell populations in the murine tumor model (FIG. 17A), from which seven cell types were annotated based on markers that define each population: hepatocytes, cancer cells (MC38), Kupffer cells, liver sinusoidal endothelial cells (LSECs), mesenchymal stem cells (MSCs), lymphocytes, and myeloid cells (FIG. 12A). The annotation of MC38 tumor cells was supported by a high correlation of chromosomal copy numbers estimated from XYZeq scRNA-seq data and publicly available MC38 cytogenetic data (Pearson r = 0.78) (25). Notably, a partial amplification of chromosome 15 and a partial deletion of chromosome 14 observed in the XYZeq data were consistent with common chromosomal abnormalities seen in MC38 cells (FIG. 17B). As a negative control, we saw low chromosomal copy number correlation when comparing MC38 cells to hepatocytes (26) and immune cells (21) (Pearson r = 0.05 and r = 0.17 respectively) (FIG. 17B). A heatmap showing differentially expressed genes across seven cell types uncovered distinct clusters of cells defined by expression of canonical genes that are relatively exclusive to each cell type (FIG. 12B). Note, we estimated uniformly low rates of contamination of each cell cluster (median under 1%) with the exception of hepatocytes, which had a slightly higher rate at 2.2% (FIG. 17C and Methods). We found comparable median UMIs and genes detected across all cell clusters including immune cell populations that have been difficult to profile using other combinatorial indexing methods (27) (FIG. 17D and FIG. 17E). Cell types expected in non-tumor bearing liver were identified using markers previously described, which included hepatocytes, Kupffer cells, and LSECs (26). Consistent with the known heterogeneity of hepatocytes, we identified hepatocyte subsets annotated by the expression of pericentral markers (Glul, Oat and Gulo) (26) (FIG. 17F). MC38 adenocarcinoma cells comprised a large uniform cluster and were distinguished by the expression of the known marker Plec (22). Myeloid cells were defined by canonical markers Cd11b and Cd74 (28), but other non-canonical markers were also observed, including Myolf (29) and Tgfb (30). Lymphocytes showed a similar mix of broad and specific expression patterns of cell type markers, with expression of pan-lymphocyte marker Il18r1, T-lymphocyte marker Prkcq and cytotoxic T-cell marker Cd8b (31-33). Lastly, we detected a cluster of mesenchymal stem/stromal cells which expressed both broad mesenchymal cell markers Rbms3 and Tshz2 and stem/stromal cell markers Prkgl and Gpc6 (34-38) (FIG. 17F).

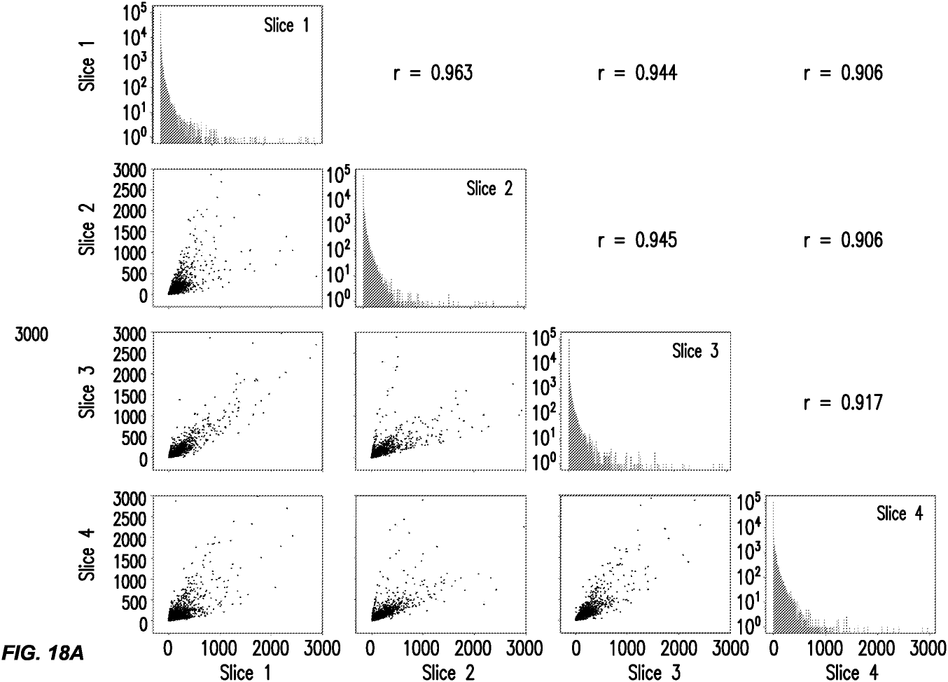

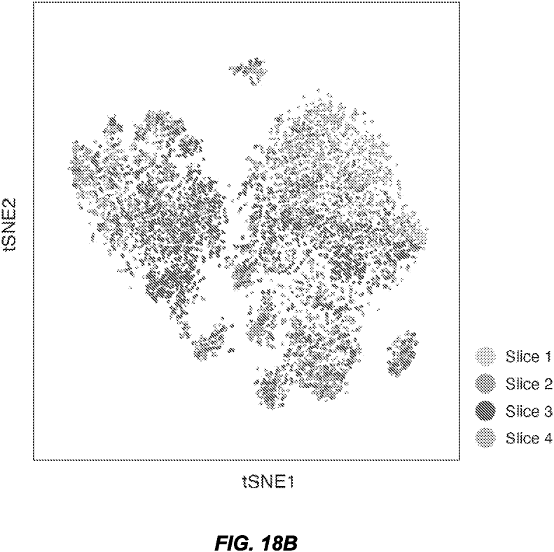

We next assessed the reproducibility of XYZeq while comparing changes in the transcriptional landscape across the z-layer of the organ. Four non-sequential 25 µm tissue slices from the same frozen liver/tumor sample block were processed and analyzed. The average expression over all cells for genes detected across all slices were highly correlated between each pair of slices (average pairwise Spearman r = 0.93) (FIG. 18A). We noted that among the four tissue sections, slice 1 and slice 2, which were the two most proximal slices in their z-coordinates (separated by 80 µm), had the highest expression correlation (Spearman r = 0.96). In contrast, slice 1 and slice 4, which were the most distal in z-coordinates (separated by 830 µm), had the lowest correlations (Spearman r = 0.91). Further, clusters jointly annotated across all four slices consisted of cells from each slice, suggesting the observed heterogeneity is not due to batch effects (FIG. 18B).

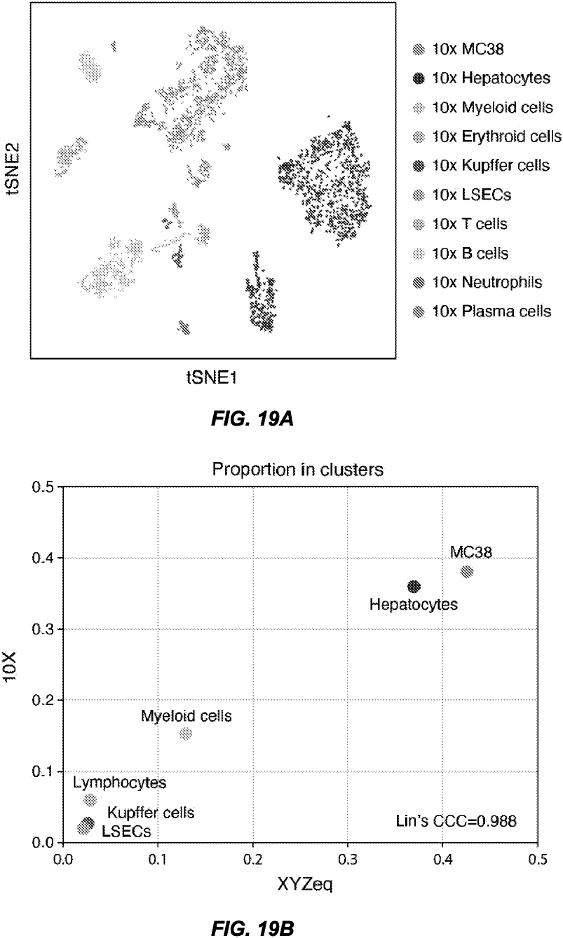

We further compared the quality of the scRNA-seq data generated by XYZeq to another single cell technology that is commercially available. To accomplish this, we compared the cell type clusters identified from XYZeq to those identified from an independent scRNA-seq dataset of the same liver/tumor model generated using the 10X Genomics droplet-based Chromium system. Most cell populations detected by 10X were also observed by XYZeq, except neutrophils, erythroid progenitors, and plasma cells (FIG. 12C and FIG. 19A), immune cell populations known to be sensitive to the cryopreservation (39) required for XYZeq. Interestingly, 10X did not capture MSCs even though cells were isolated from fresh liver/tumor samples. In addition, B cells identified using the 10X platform correlated with the myeloid population detected by XYZeq, likely due to the transcript capture of Ly86, Cd74 and several Class II histocompatibility antigen genes (e.g. H2ab1 or H2dmb1). For the six cell types identified in both the 10X and XYZeq data, we observed high correlations in both the cell-type proportions (Lin’s CCC = 0.99; FIG. 19B) and the pseudobulk expression profiles of each cell type (Pearson r = 0.64 - 0.86, p < 0.01, FIG. 12C).

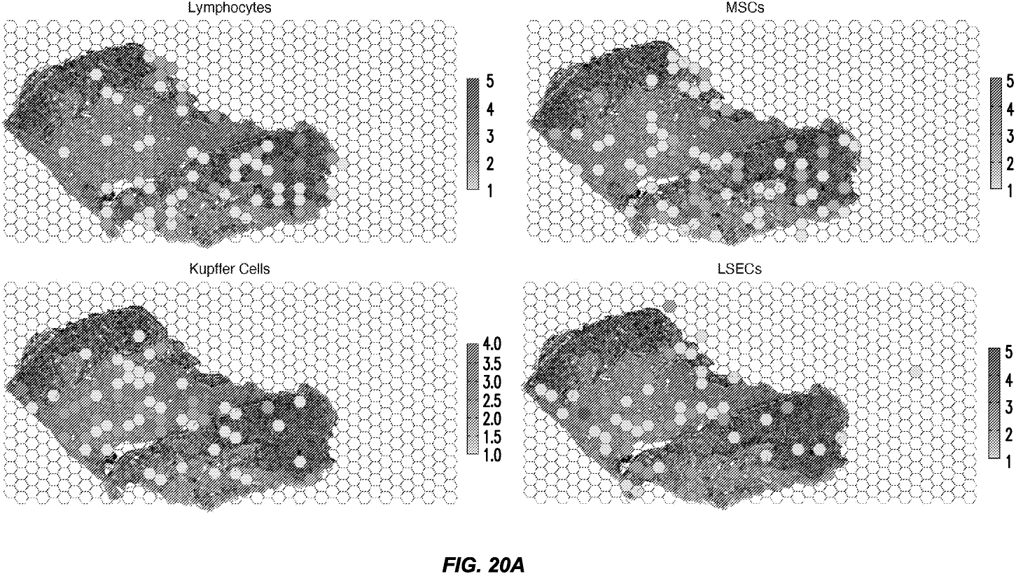

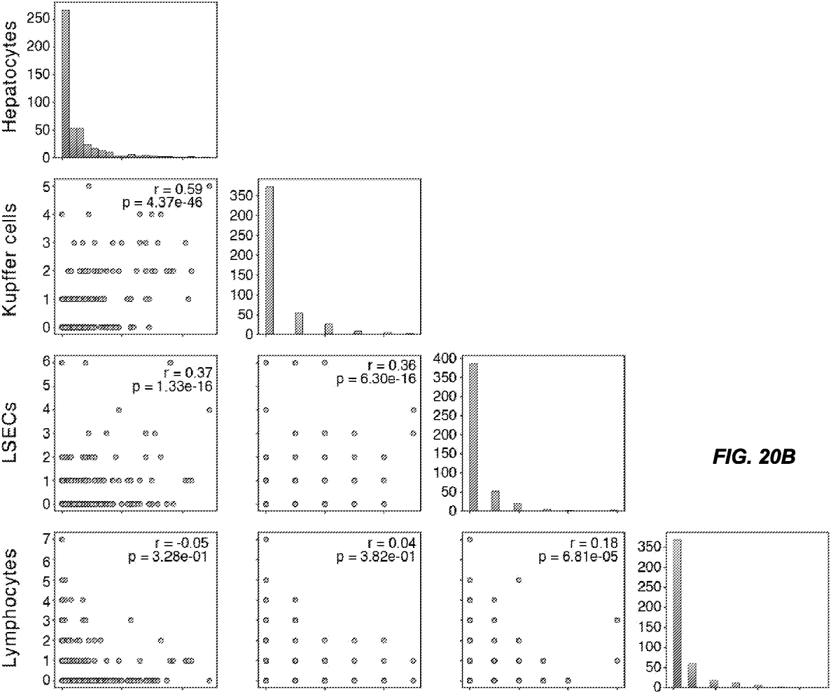

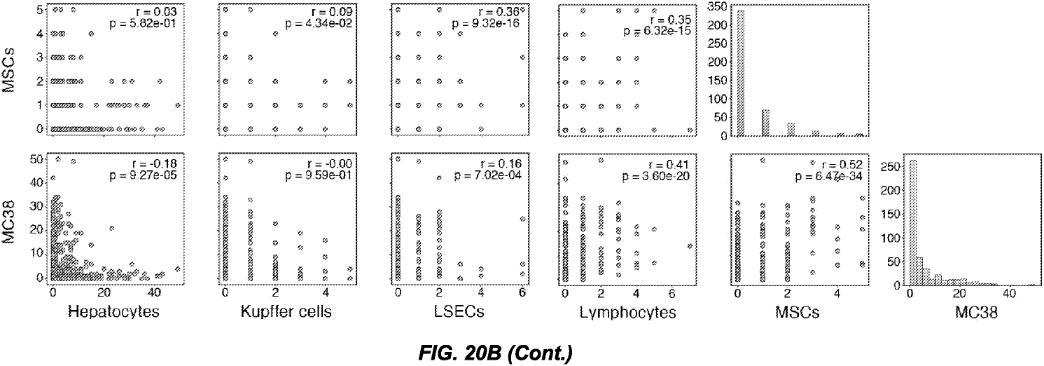

Next, we turned to the critical question of whether XYZeq can determine the spatial location of each cell. To do this, we compared the spatial localization of each cell cluster to the images of H&E-stained sequential slices. First to determine that we could accurately define liver from tumor tissue, we confirmed that the density of hepatocytes and cancer cells across the spatial wells overlap with the histological annotation of the adjacent section (FIG. 12D). Projection of other cell types revealed distinct spatial organization patterns for myeloid cells, lymphocytes, Kupffer cells, MSCs and LSECs (FIG. 12D and FIG. 20A). Quantification of cellular composition occupying each spatial well revealed MSCs, lymphocytes, and myeloid cells were co-localized with cancer cells, while Kupffer cells and LSECs co-localized with hepatocytes, suggesting potential regions of cellular interaction in tumor infiltrated tissue (FIG. 12E and Methods). These qualitative observations were confirmed by pairwise correlation analysis of cell type proportion across all the wells (0.37≤ Pearson r≤0.77, p<0.05; FIG. 12F and FIG. 20B).

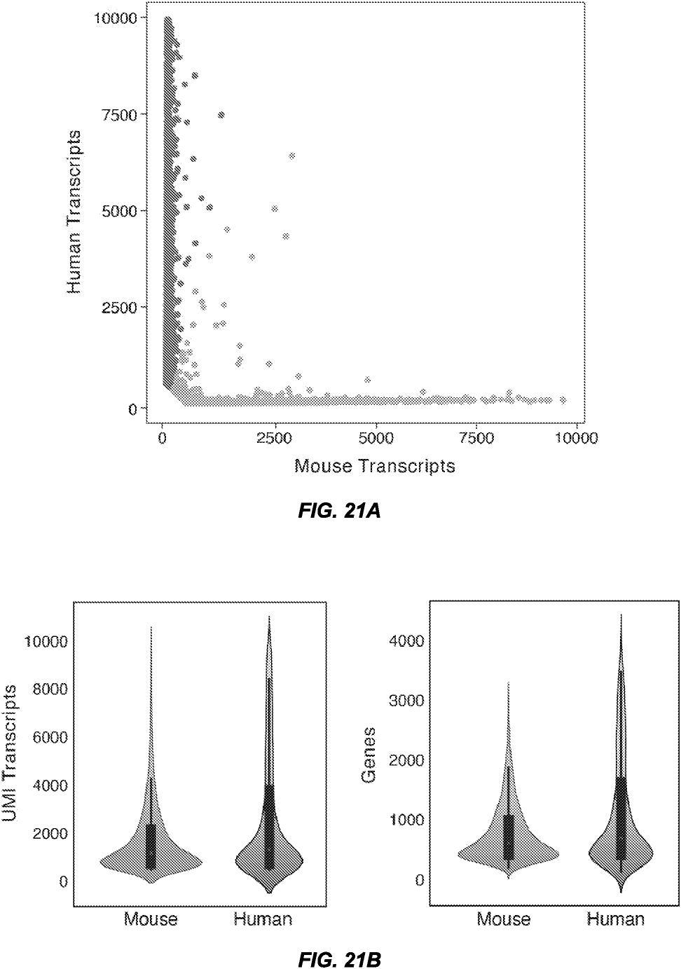

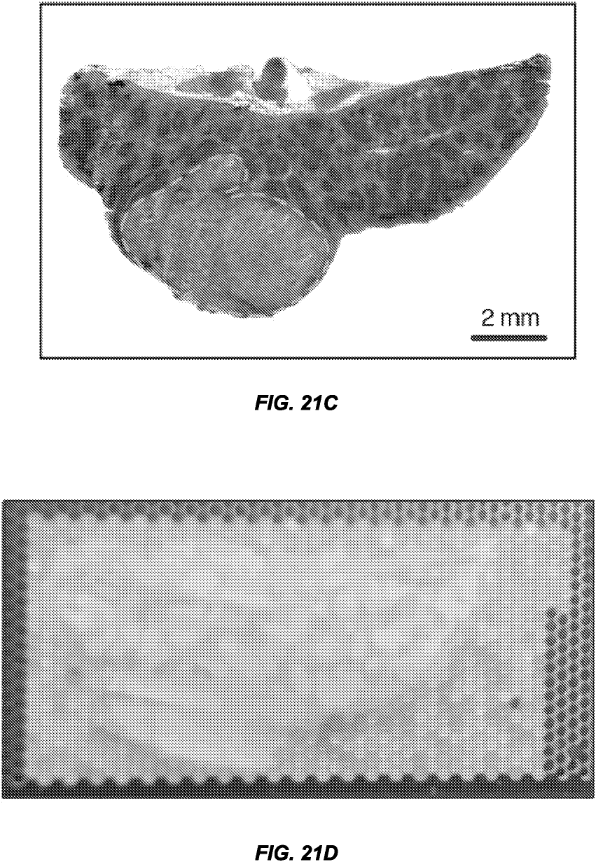

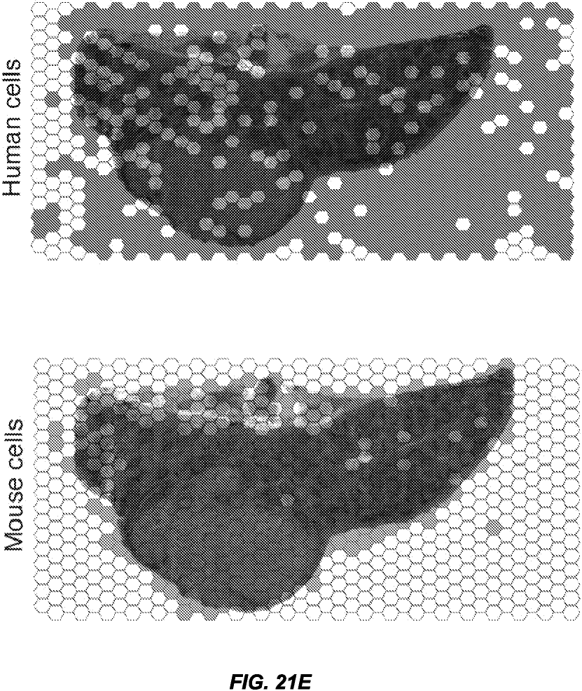

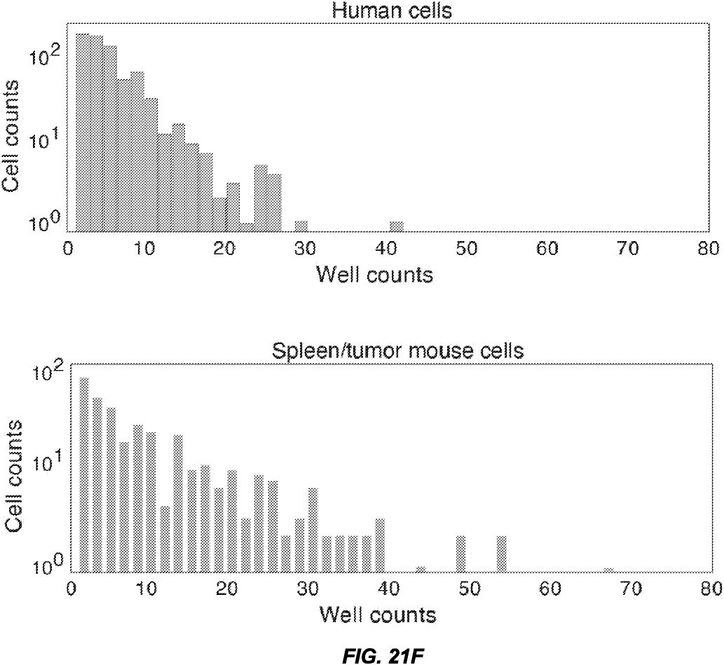

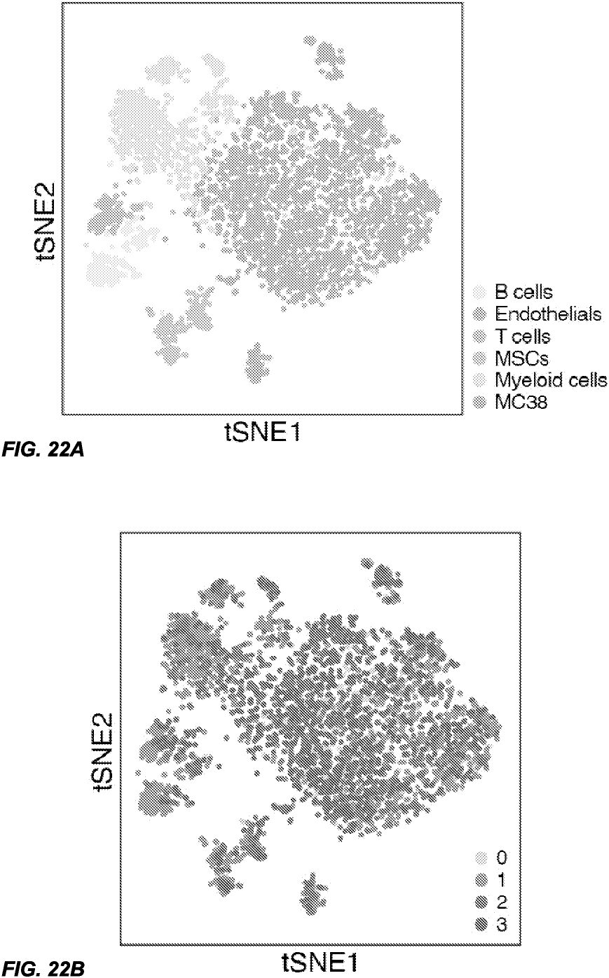

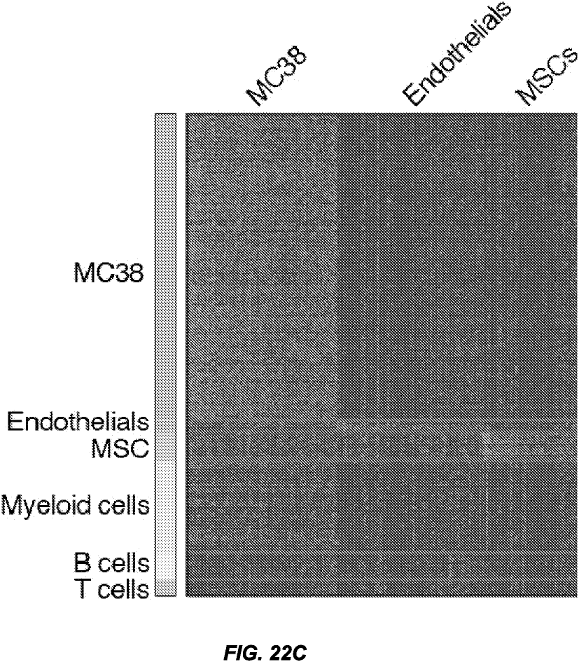

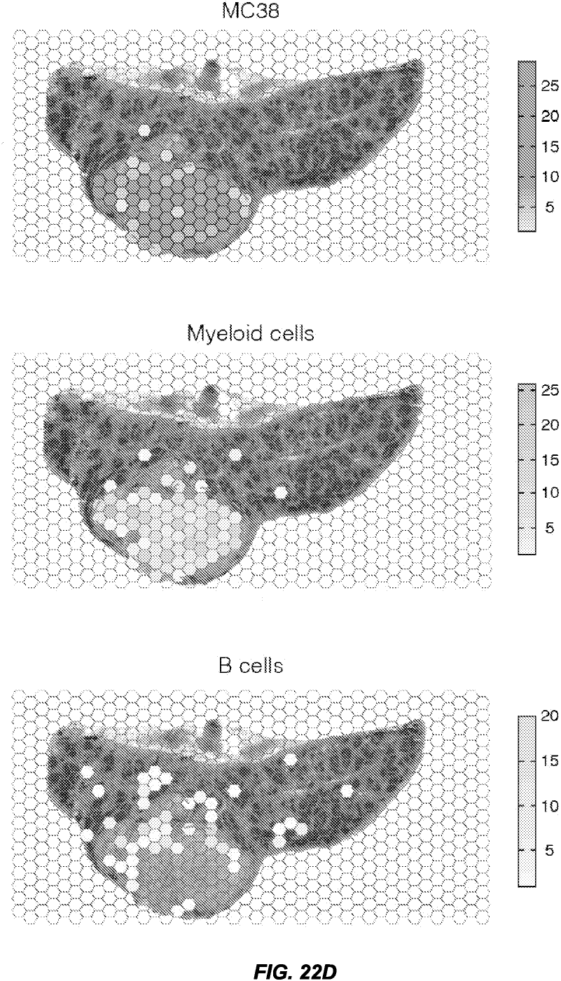

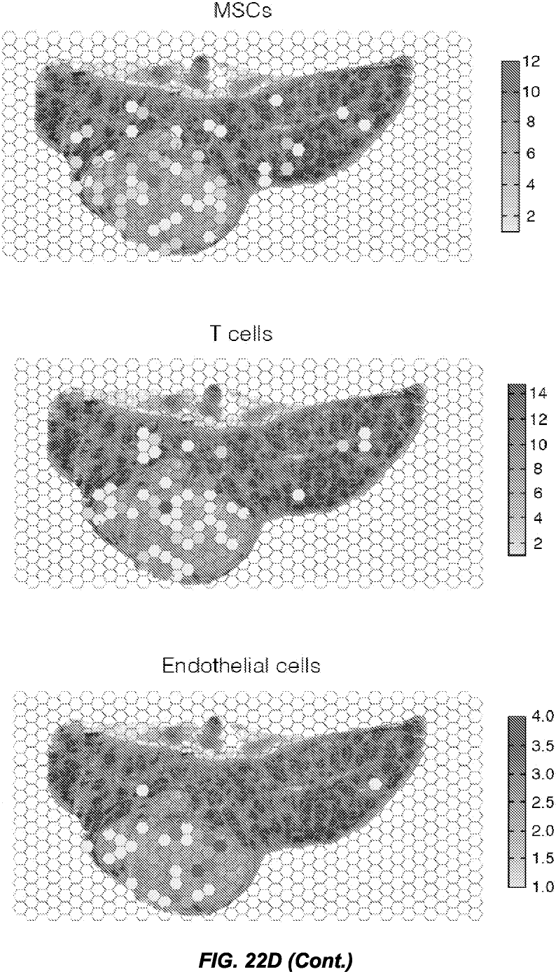

To assess the generalizability of XYZeq to other tissues, we processed samples from the same heterotopic murine tumor model in the spleen. We recovered a total of 7,505 cells at a median of 1,312 UMIs and 661 unique genes per HEK293T cell and 1,169 UMIs and 577 unique genes per mouse cell at an estimated collision rate of 1.36% (FIG. 21A and FIG. 21B). Similar to the liver/tumor model, XYZeq was able to reconstruct the boundaries of the splenic mouse tissue with the MC38 tumor region annotated on a sequential H&E-stained slice (FIG. 21C to FIG. 21E). A median of 4 human cells/well and 7 mouse cells/well were detected (FIG. 21F). Semi-supervised Leiden clustering revealed six distinct cell populations for the spleen/tumor model including: B cells, T cells, myeloid cells, MSCs, endothelial cells, and MC38 tumor cells (FIG. 22A). We observed that all four spleen/tumor slices contributed to each cell type cluster, suggesting that the annotated clusters are not due to batch effects (FIG. 22B). A heatmap showing differentially expressed genes across the six cell types revealed distinct clusters of cells expressing canonical genes that are relatively exclusive to each type (FIG. 22C). Cells from each type could be spatially mapped across the tissue (FIG. 22D). Collectively, these results demonstrate that XYZeq can generate spatially resolved single cell RNA-seq data from different fixed frozen tissues.

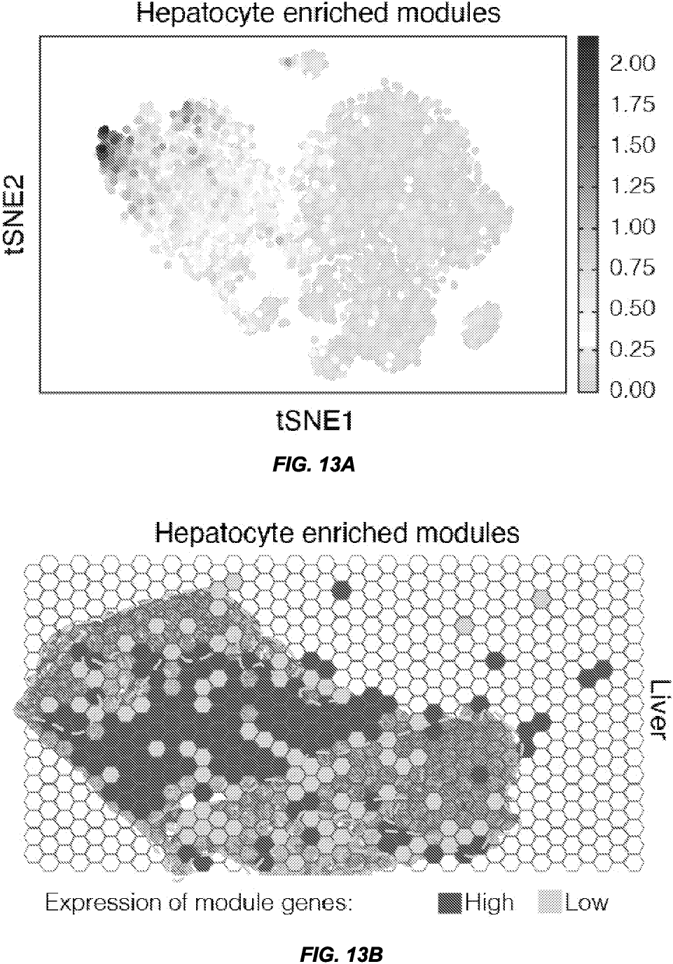

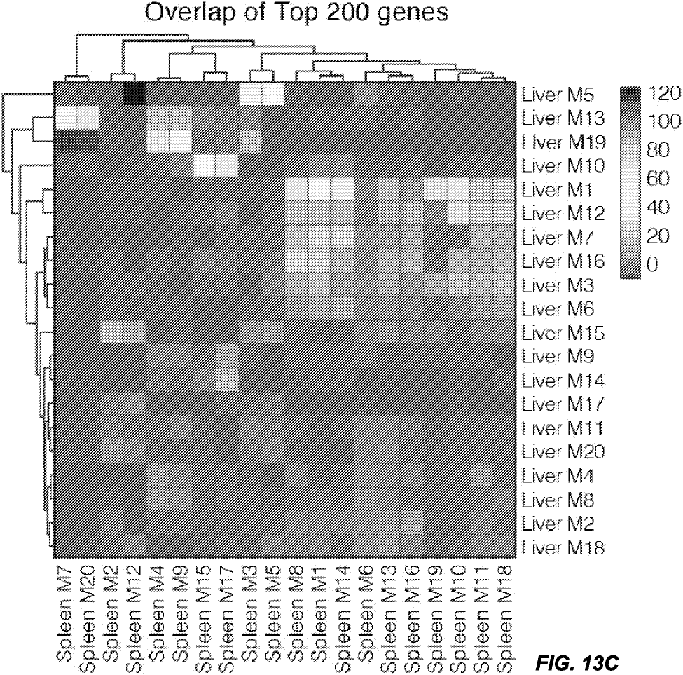

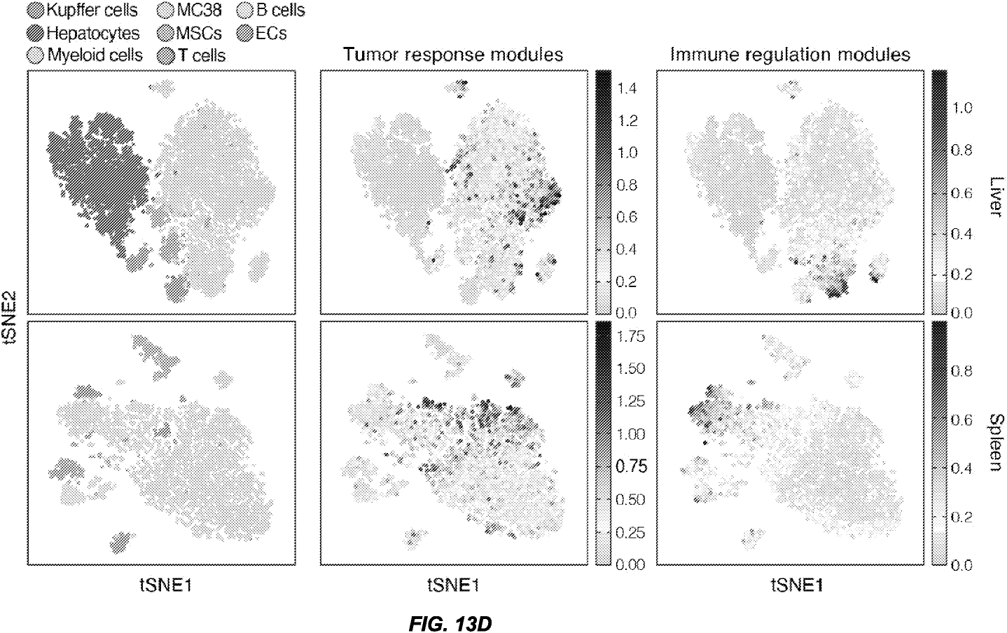

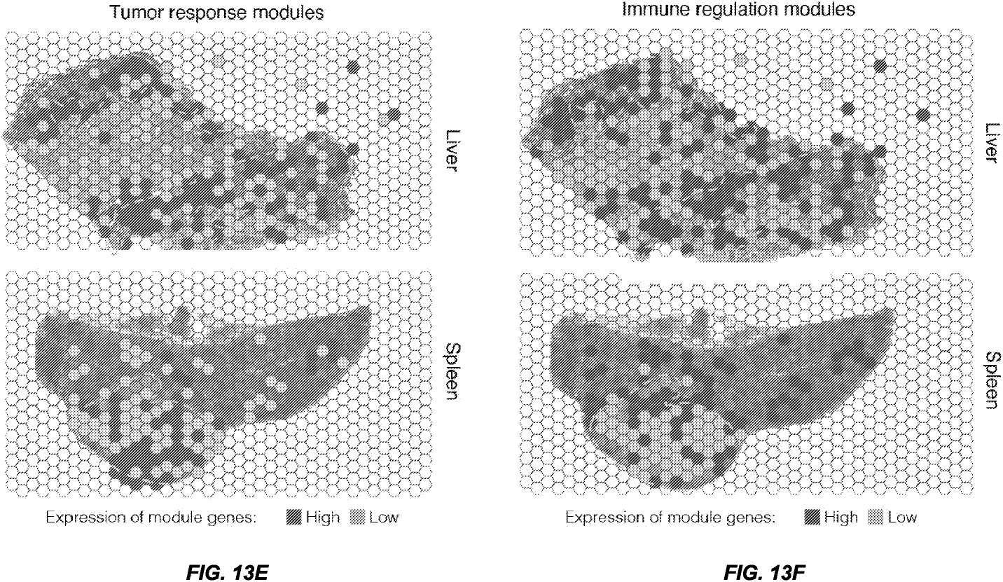

The ability to obtain spatial and single-cell transcriptomic data simultaneously allowed us to assess the effects of cellular composition on gene expression patterns across space. We applied non-negative matrix factorization (NMF) to both the liver/tumor and spleen/tumor scRNA-seq data to define modules of co-expressed genes and associated the expression of each module in each cell type with its expression across spatial wells. Using our approach, we identified twenty modules of co-expressed genes in each tissue (Methods). As a proof of principle of the approach, we first identified liver module (LM) 14 from the liver/tumor data, which was predominantly expressed by the hepatocyte cluster in the tSNE space (FIG. 13A). As expected, the highest LM14 expressing wells were enriched for hepatocytes suggesting that the spatial variability of this module is largely driven by the frequency of hepatocytes (FIG. 13B).

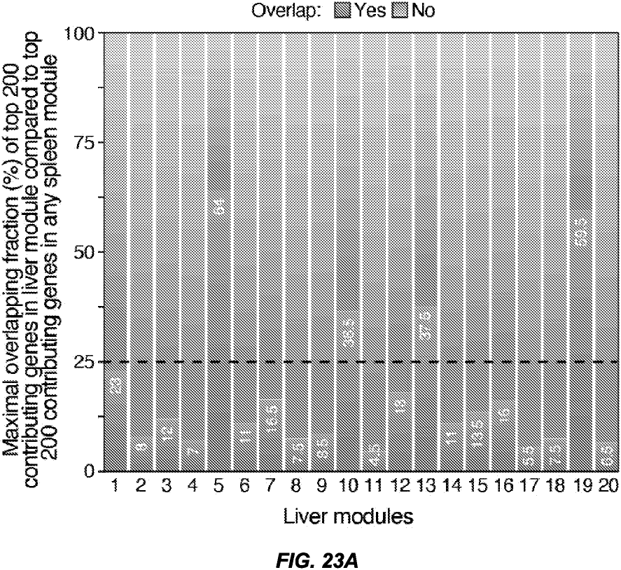

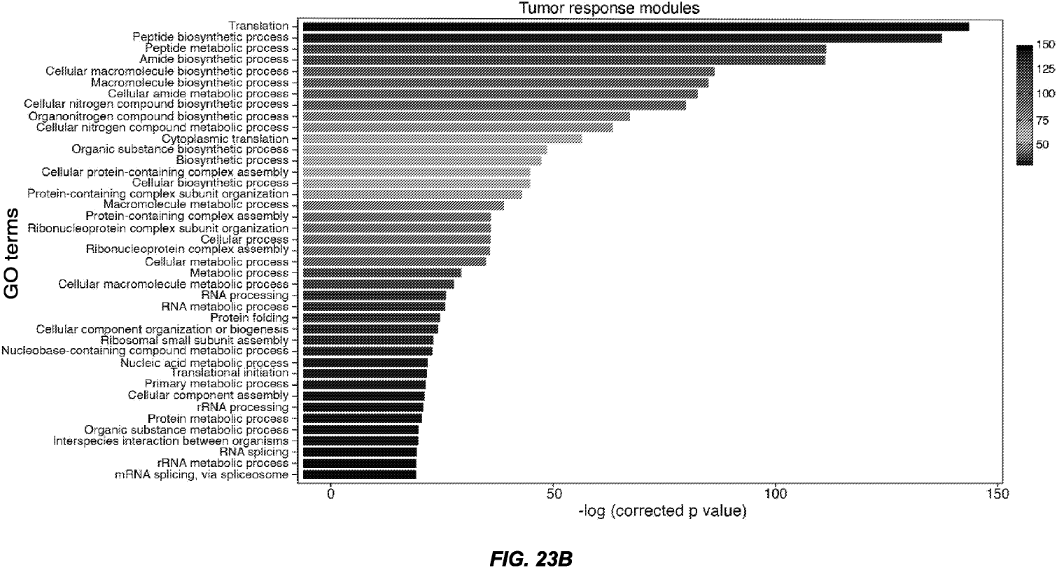

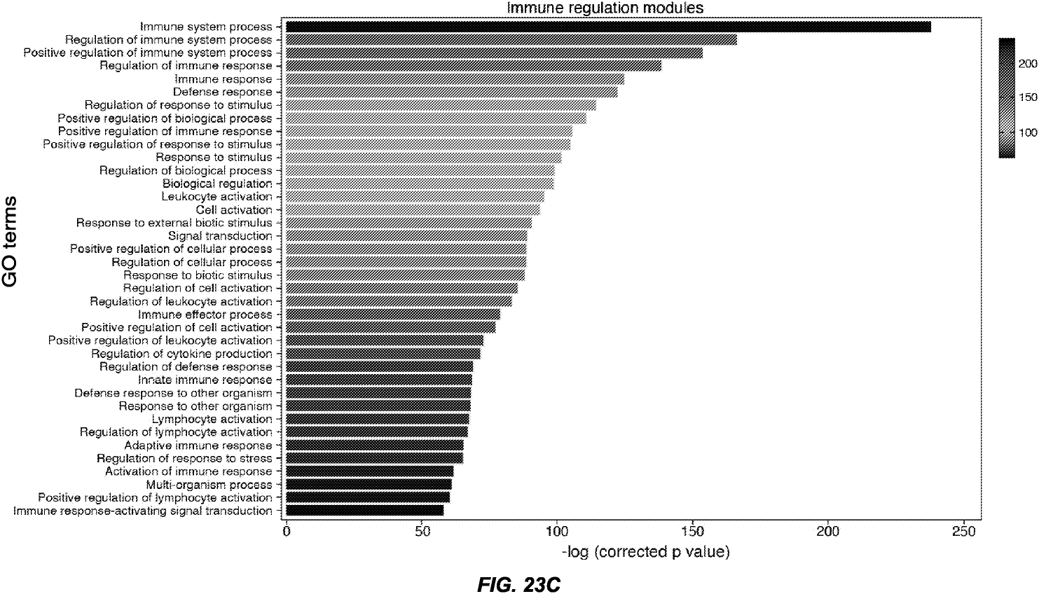

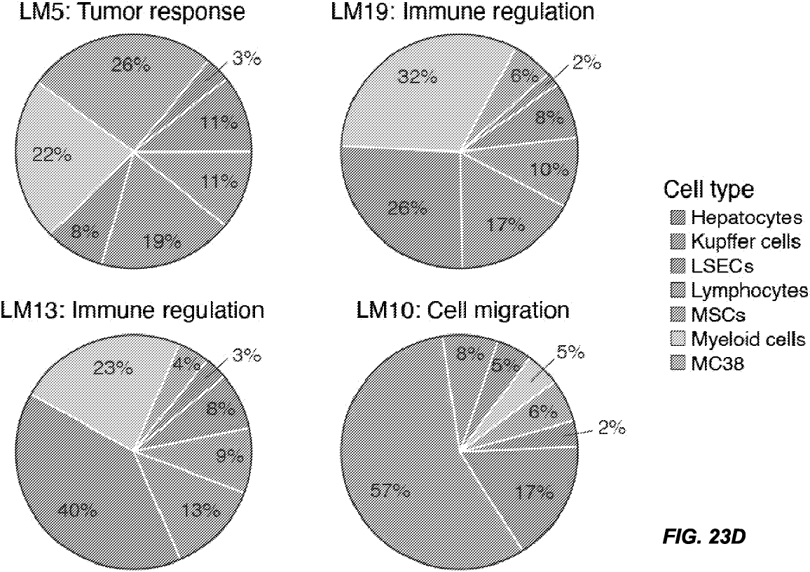

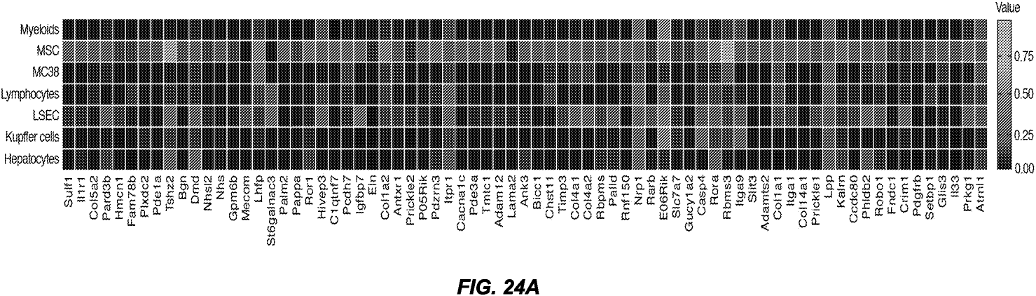

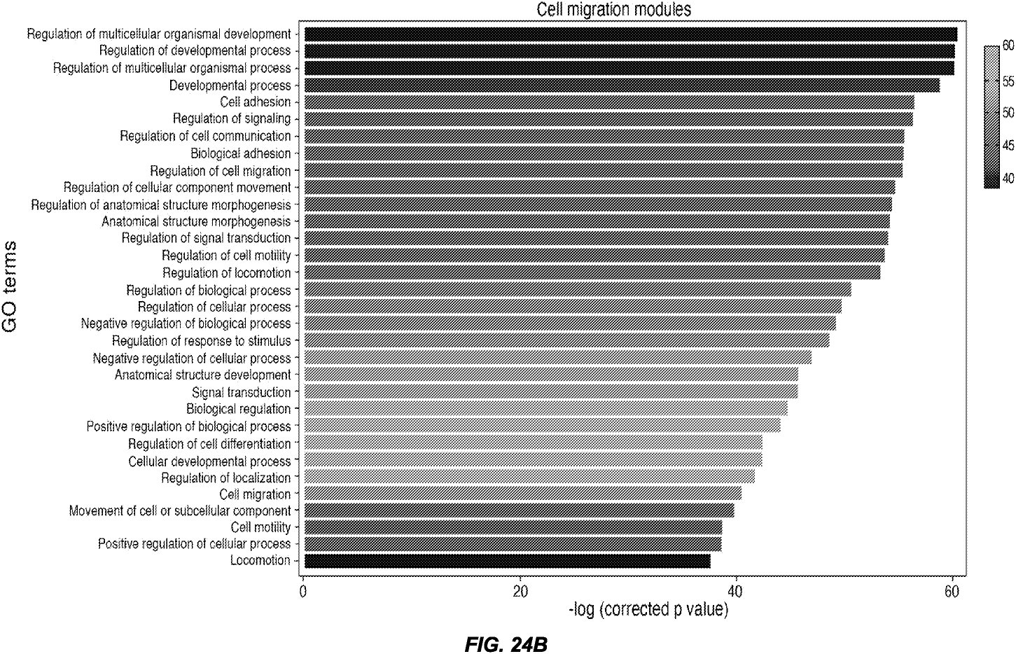

Next, we reasoned that because both the liver and spleen were injected with the same tumor cell line, the invading tumors may induce a shared gene expression profile that vary over space, driven in part, by the cellular composition of the tumor microenvironment. To test this hypothesis, we first identified pairs of matching gene modules between the two tissues from the NMF analysis (Methods). We found four distinct liver modules (LM) that had at least 25% of genes overlapping with spleen/tumor modules (SM) (FIG. 13C and FIG. 23A). Gene Ontology (GO) analysis of the modules revealed the enrichment of genes implicated in tumor-response, immune regulation, and cell migration (FIG. 23B and FIG. 23C; and FIG. 24B). Consistent with the enrichment analysis, many of the genes from these modules have been implicated in tumorogenesis (complete gene lists in Table 3). Unlike LM14, further analysis of these matching modules revealed a heterogeneous composition of cell populations that contributed to the expression of specific module genes (FIG. 23D and Methods). For example, the tumor response module LM5 and its matching modules SM2 and SM12 (FIG. 13C and FIG. 23A), consisted of genes predominantly expressed in MC38 tumor cells with some expression in myeloid cells and lymphocytes (FIG. 13D; FIG. 23D; and Methods). The immune regulation modules, LM13 and LM19 (matched with SM7 and SM20), consisted of genes expressed primarily in both conventional (e.g. myeloid and lymphocytes) and nonconventional (e.g., Kupffer cells from liver samples) immune cells (FIG. 13C and FIG. 13D; and FIG. 23D). The expression of these overlapping modules were highest in regions densely infiltrated with cancer cells (FIG. 13E and FIG. 13F). Collectively, these results show that the joint analysis of scRNA-seq and spatial metadata from XYZeq can identify spatially variable gene modules due to differences in cellular composition across tissue samples.

TABLE 3

Lists of overlapping genes among top 200 contributing genes between liver and spleen.

| | |
|---|---|
| Modules | Overlapping genes |
| Tumor response modules | Lgals1 Rpl8 Rpsa Ly6e Rps12 S100a4 Rplp1 Tapbp S100a6 Rpl26 Ptma Rpl14 Rpl38 Rps24 Rps8 Vim Hsp90ab1 B2m Gnb2l1 Rpl21 Ptrf Rplp0 |
| (128 genes, LM5) | Col3a1 Rpl30 Rps20 Rps26 Rps19 Npm1 Rpl13 Rpl37 Rps5 Rps29 Ly6a Rpl32 Pfn1 Rps15 Rps15a Pfdn5 Ncl Tubalc Rpl23a Ahnak Rpl13a Rps21 Arpclb Eeflg Rpl41 Cct7 Rpl37a Rpl23 Calm1 Myh9 Psmc5 Hdgf Msn Pkm Rpl12 Fau Rps27 Rps14 Serpinh1 Cd63 Rpl9 Pdia3 Eef2 Rpl17 Arhgdia Cfl1 Rpl4 Rpn2 Edf1 Ccnd1 Ftl1 Sparc Rps25 Hspa8 Rplp2 Plec Rps10 Scd2 Cyb5r3 Serbp1 Rps17 Cct5 Rcn1 Rpl5 Rpl19 H1f0 Grn Rps27a Rpl11 Tuba1a P4hb Rpl36 Hnmpab Rpl36a Pabpc1 Eif5a Eef1b2 Hspa9 Bsg Rpl27 Rps3 Rpl22 Ybx1 Psmb10 Tmsb10 Col6a1 Ecm1 Fth1 Set Cald1 Canx Tubb5 Cox4i1 Vcp Eef1a1 S100a10 Rpl34 Ldha Gabarap Rpl18a Hint1 Ppplca Kpnb1 Tcp1 Rps28 Pdap1 |
| Immune regulation modules (75 genes, LM13) | Ptprj Fyb Mertk Aoah Adap2 Pou2f2 Adgre1 Myo9a Unc93b1 Dock10 Frmd4b Zdhhc14 Myolf Prkcb Zeb2 Tmcc3 Zfp710 Slc9a9 Itga9 Lcp2 Hck Fcer1g Mrc1 Rreb1 Spi1 Ccr5 1700112E06Rik Elmo1 March1 Abcg3 Trpm2 Lyn Gm5150 Slc8a1 Nrros Ctsc Gab2 Sirpa Ly86 P2rx4 Pla2g4a Entpd1 Cd84 Fgr Acer3 Cd300lf Cadm1 Tmsb4x Msr1 Maf Mitf Lgmn Csf1r Ncf2 Ptprc Gmip Pik3ap1 Ctsb Lacc1 Arhgap15 Dock8 Plcl2 Shtn1 Prex1 Diaph2 Exoc6 Dock4 Mgat5 Slc43a2 Arrb2 Ppm1h Slc15a3 Pld4 Tbxas1 Vrk2 |
| Immune regulation modules (119 genes, LM19) | Tgfbi Cd74 Arhgap15 Ccr5 Ctss Ly86 Myolf 1700112E06Rik Dock10 Elmo1 Tgfbr1 Inpp5d Epsti1 Zeb2 Ccr2 H2-Ab1 Ptprc Slc8a1 Dock2 Dock8 Laptm5 Arhgap30 Hck Ms4a6c Cd84 Lyn Ctsc Cyth4 Fyb Itga4 Pld4 Gab2 Lcp1 Cybb Unc93b1 Tbxas1 Gpr141 Hmha1 C1qa Tmsb4x Plcg2 March1 Cx3cr1 Gm4955 Ctsb Pde7b Cd300lf Sirpa Dock4 Zfp710 Nlrp1b Pik3cd Fam49b Klra2 Ms4a6b Slfn5 H2-Eb1 Aoah Lcp2 Csf1r Arhgap24 Abr Cers6 Entpd1 H2-AaPtk2b Sp100 H2-DMb1 Slfn2 Mpeg1 Aim2 Apobec1 Rreb1 Etv6 Adap2 Irf7 Spi1 Ms4a4c Gm2a Lyz2 Lgmn Stat1 Ms4a7 Tnfrsf11a Apba1 Pik3ap1 Fam105a Nckap1l Psap Arid5b Mndal Slfn8 Trim30a Lair1 Picalm Il10ra Ncf1 Ccdc88b Corola C1qc Gatm Tgm2 Ctsh C1qb Pip4k2a Chd7 Mx1 Samhd1 Fcgr2b Diaph2 Mitf Mef2a Pou2f2 Pid1 Ifi203 Wdfy4 Fnbp1 Dennd1b Pik3r5 |
| Cell migration modules (73 genes, LM10) | Tshz2 Col1a2 Rbms3 Igfbp7 Kalrn Prkg1 Ccdc80 Rora Palm2 Timp3 Pdzm3 Pde3a Adamts2 Ror1 Sulf1 Palld Pcdh7 C1qtnf7 Tmtc1 Pde1a Slit3 Adam12 Col14a1 Col5a2 Casp4 Col1a1 Ank3 Bicc1 Col4a2 Plxdc2 Il1r1 1700112E06Rik Lhfp Rnf150 Pard3b Cacna1c Crim1 Itga1 Itga9 9530026P05Rik Gucy1a2 Col4a1 Pdgfrb Nhs Hivep3 Fndc1 Prickle1 Chst11 Rarb Robo1 Il33 Gpm6b Itpr1 Bgn Mecom Slc7a7 Atrnl1 Rbpms Hmcn1 Pappa Lpp Phldb2 Dmd Antxr1 Lama2 St6galnac3 Fam78b Nrp1 Setbp1 Eln Nhsl2 Prickle2 Glis3 |

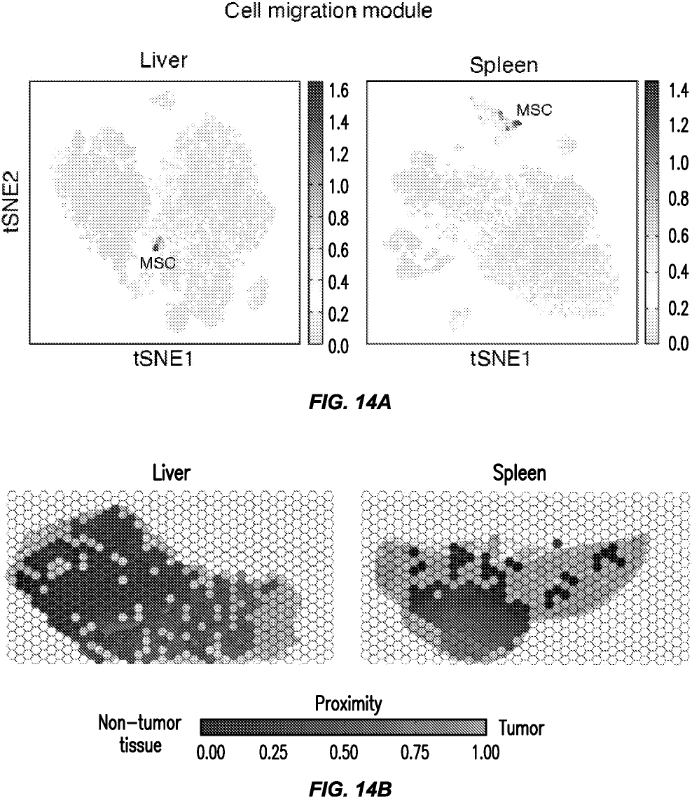

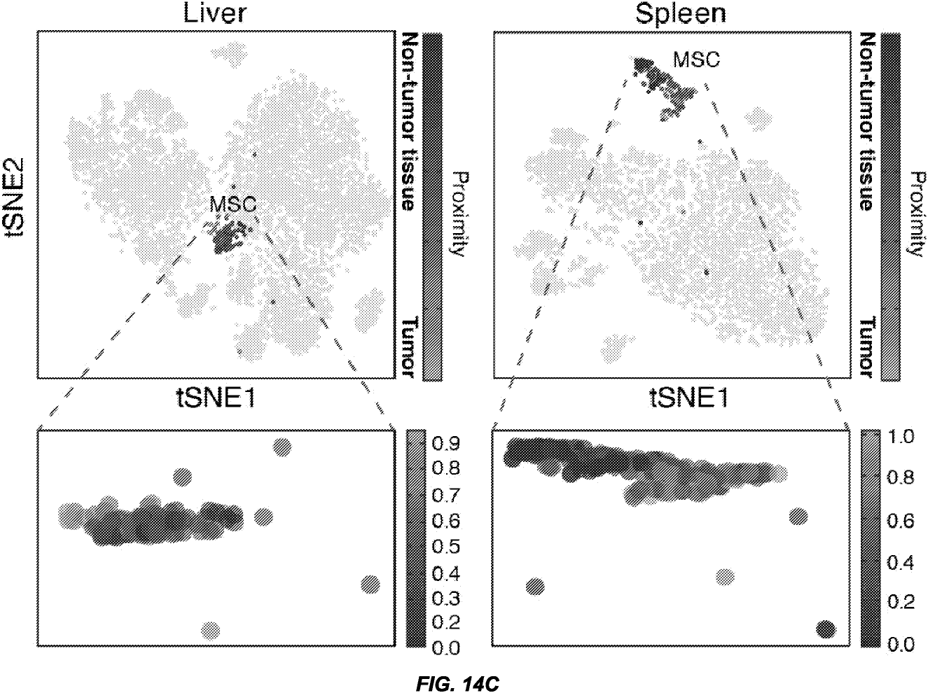

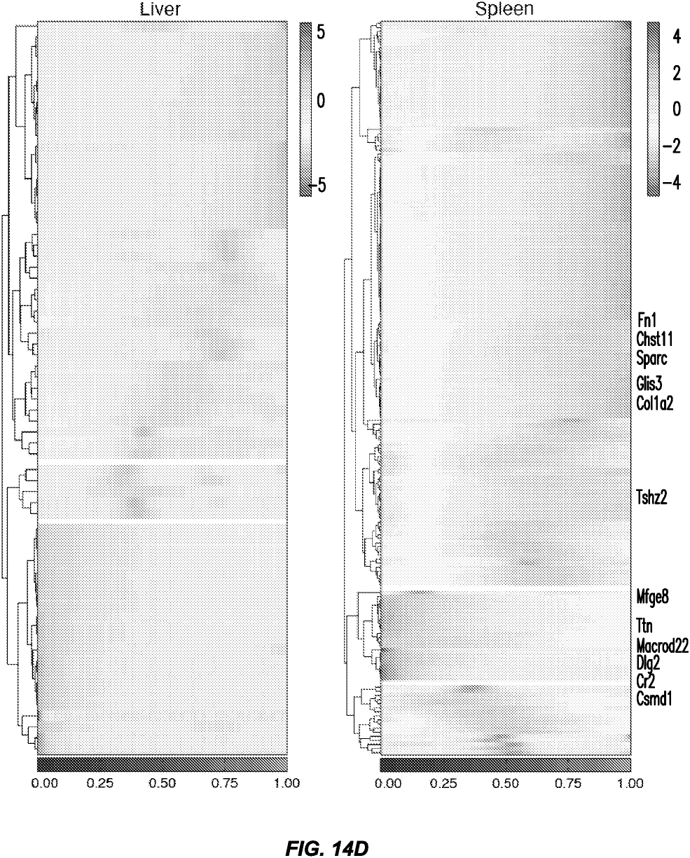

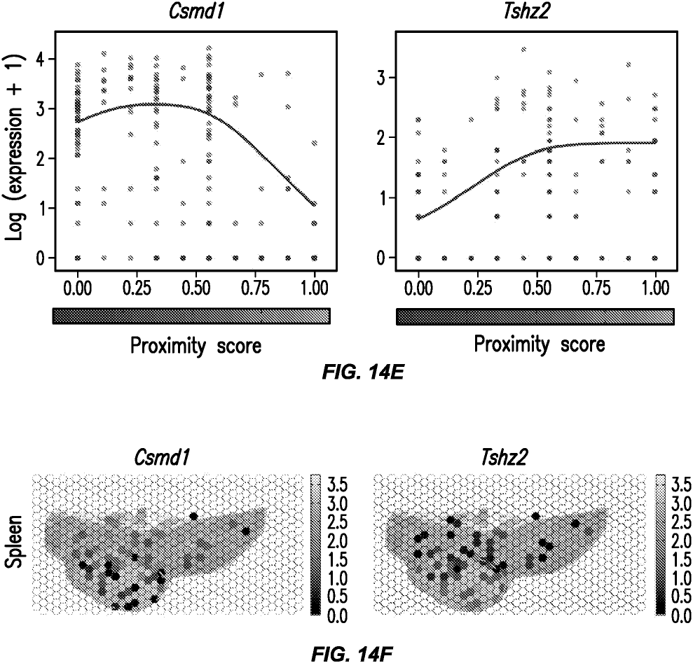

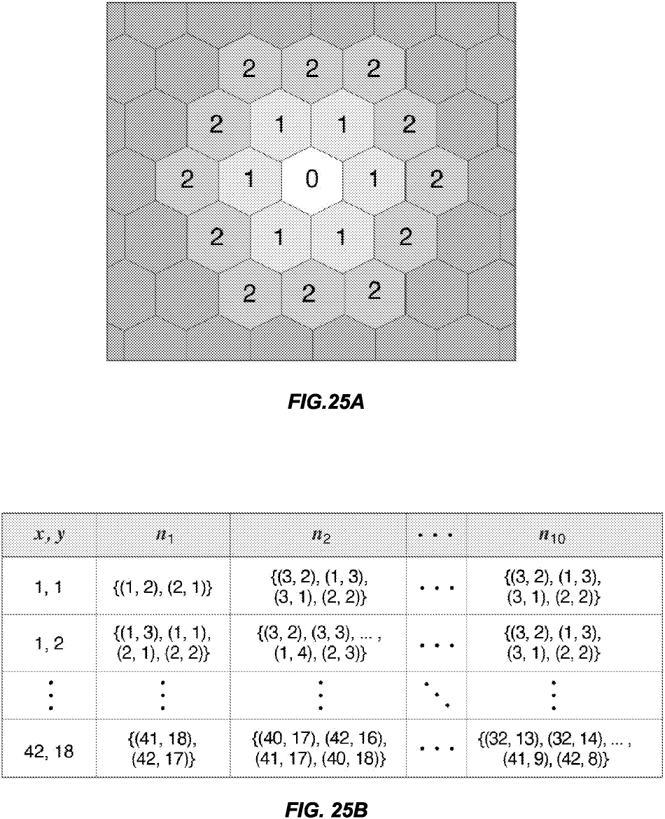

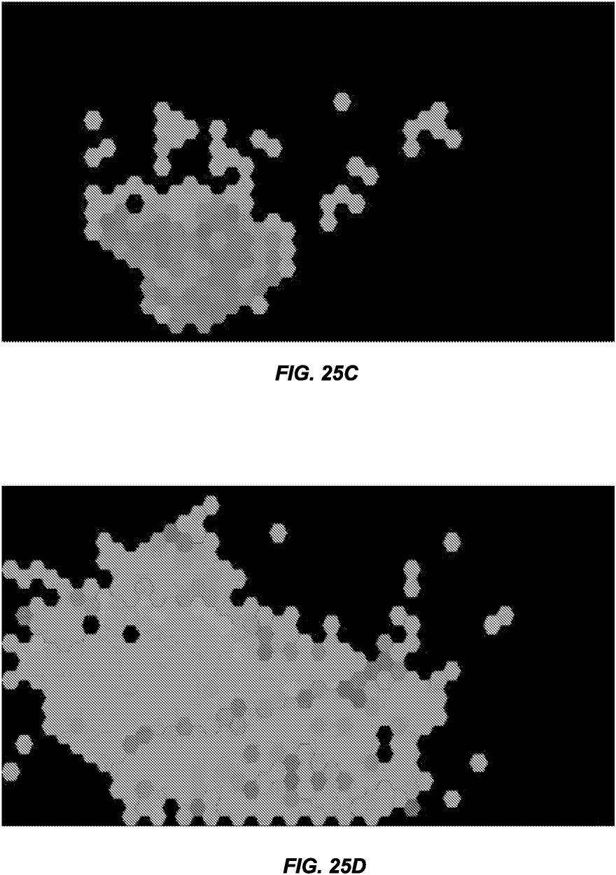

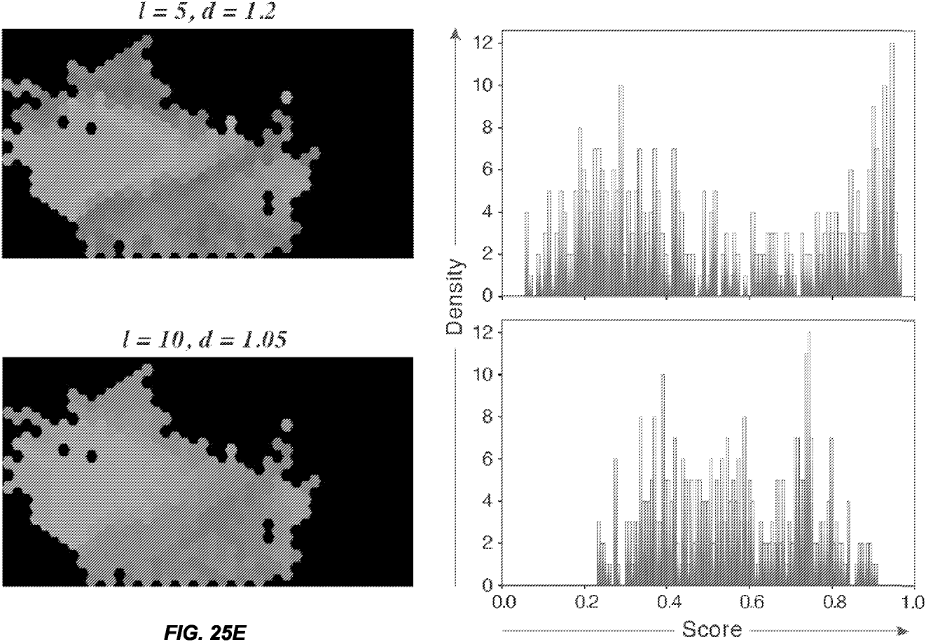

We next focused our analysis on matching modules LM10 and SM15/SM17, which are primarily expressed by MSCs and enriched for genes involved in cell migration (FIG. 13C; FIG. 14A; FIG. 23D; FIG. 24A; and FIG. 24B). Because MSCs are known to possess homing abilities to injured or inflamed sites (40), we hypothesized that LM10 could be differentially expressed in MSCs based on their proximity to the tumor. To test this hypothesis, we first computed a tumor proximity score for each well based on the composition of and distance from nearby wells (FIG. 14B; see Methods and FIG. 25 for score definition). Projecting the proximity score onto MSCs in tSNE space revealed that the transcriptional heterogeneity of the population is associated with spatial proximity to tumor (FIG. 14C). We then analyzed the MSC expression profiles using tradeSeq (41) to identify differentially expressed genes that tracked with the proximity score. We identified and clustered 177 genes from the liver/tumor tissue (p < 0.05) and 66 genes from the spleen/tumor tissue (p < 0.05) that are associated with the continuous, one-dimensional proximity score (FIG. 14D). The genes were broadly divided into three groups based on the proximity cells to tumor: intra-tumor, tumor-tissue boundary, and intra-tissue with statistically significant genes highlighted for the spleen/tumor tissue (Benjamini-Hochberg FDR < 0.05) (FIG. 14D). Interestingly, for MSCs found in the intratumor regions of the spleen/tumor, many of the differentially expressed genes are reported to regulate the extracellular matrix (ECM) (FIG. 14D, right panel) (42-45), suggesting that MC38 cells may induce a local gene expression program in neighboring MSCs that could contribute to malignant remodeling of the ECM.

Finally, we leveraged the scRNA-seq data from XYZeq to visualize how individual MSCs expressed Tshz2 and Csmd1, two genes of divergent function that are spatially variable with respect to the tumor in the spleen. Both genes are characterized as tumor suppressor genes and are often silenced in cancer cells to promote malignant growth and metastasis (36, 46, 47). However, we found spleen/tumor MSCs expressed lower levels of Csmd1 but higher levels of Tshz2 in closer proximity to the tumor (FIG. 14E). Importantly, the mean differential expression of these genes was specific to splenic MSCs and not expressed by MC38 tumor cells. The expression pattern of each of these genes in space revealed a pattern consistent with the aforementioned spatial trajectory analysis, suggesting that their heterogeneous expression in MSCs may be determined by the location of the cells with respect to tumor (FIG. 14F). Taken together, these results reveal that joint analysis of spatial and single-cell transcriptomic data from XYZeq can detect transcriptionally variable genes within specific cell types (e.g. MSCs) driven by their location within the complex tissue architecture.

### 3. Discussion

We introduce XYZeq, a new single-cell RNA-sequencing workflow that encodes spatial meta information at 500 µm resolution. XYZeq enables unbiased single-cell transcriptomic analysis to capture the full spectrum of cell types and states while simultaneously placing each cell within the spatial context of complex tissue. In murine tumor models, we demonstrate that XYZeq identifies both spatially variable patterns of gene expression determined by cellular composition and heterogeneity within a cell type determined by spatial proximity. Looking forward, XYZeq provides a scalable workflow that can be adapted to multiple z-layers of tissue and can potentially facilitate analysis of entire organs. Large scale integrated profiling of multiple modalities of single cells mapped to the structural features of their tissue will enable greater understanding of how the tissue microenvironment affect cellular infiltration and interaction in health and disease.

### REFERENCES

1. A. P. Patel et al., Single-cell RNA-seq highlights intratumoral heterogeneity in primary glioblastoma. Science 344, 1396-1401 (2014).

2. S. V. Puram et al., Single-Cell Transcriptomic Analysis of Primary and Metastatic Tumor Ecosystems in Head and Neck Cancer. Cell 171, 1611-1624 e1624 (2017).

3. C. Ziegenhain et al., Comparative Analysis of Single-Cell RNA Sequencing Methods. Mol Cell 65, 631-643 e634 (2017).

4. I. C. Macaulay, C. P. Ponting, T. Voet, Single-Cell Multiomics: Multiple Measurements from Single Cells. Trends Genet 33, 155-168 (2017).

5. M. L. Suva, I. Tirosh, Single-Cell RNA Sequencing in Cancer: Lessons Learned and Emerging Challenges. Mol Cell 75, 7-12 (2019).

6. V. Svensson, R. Vento-Tormo, S. A. Teichmann, Exponential scaling of single-cell RNA-seq in the past decade. Nat Protoc 13, 599-604 (2018).

7. K. H. Chen, A. N. Boettiger, J. R. Moffitt, S. Wang, X. Zhuang, RNA imaging. Spatially resolved, highly multiplexed RNA profiling in single cells. Science 348, aaa6090 (2015).

8. A. Raj, P. van den Bogaard, S. A. Rifkin, A. van Oudenaarden, S. Tyagi, Imaging individual mRNA molecules using multiple singly labeled probes. Nat Methods 5, 877-879 (2008).

9. C. L. Eng et al., Transcriptome-scale super-resolved imaging in tissues by RNA seqFISH. Nature 568, 235-239 (2019).

10. S. Shah, E. Lubeck, W. Zhou, L. Cai, seqFISH Accurately Detects Transcripts in Single Cells and Reveals Robust Spatial Organization in the Hippocampus. Neuron 94, 752-758 e751 (2017).

11. P. L. Stahl et al., Visualization and analysis of gene expression in tissue sections by spatial transcriptomics. Science 353, 78-82 (2016).

12. S. G. Rodriques et al., Slide-seq: A scalable technology for measuring genome-wide expression at high spatial resolution. Science 363, 1463-1467 (2019).

13. S. Vickovic et al., High-definition spatial transcriptomics for in situ tissue profiling. Nat Methods 16, 987-990 (2019).

14. R. R. Stickels etal., Highly sensitive spatial transcriptomics at near-cellular resolution with Slide-seqV2. Nat Biotechnol, (2020).

15. K. Achim et al., High-throughput spatial mapping of single-cell RNA-seq data to tissue of origin. Nat Biotechnol 33, 503-509 (2015).

16. R. Satija, J. A. Farrell, D. Gennert, A. F. Schier, A. Regev, Spatial reconstruction of single-cell gene expression data. Nat Biotechnol 33, 495-502 (2015).

17. J. Cao et al., Comprehensive single-cell transcriptional profiling of a multicellular organism. Science 357, 661-667 (2017).

18. A. B. Rosenberg et al., Single-cell profiling of the developing mouse brain and spinal cord with split-pool barcoding. Science 360, 176-182 (2018).

19. M. Attar et al., A practical solution for preserving single cells for RNA sequencing. Sci Rep 8, 2151 (2018).

20. S. Yang et al., Decontamination of ambient RNA in single-cell RNA-seq with DecontX. Genome Biol 21, 57 (2020).

21. J. C. Lee et al., Regulatory T cell control of systemic immunity and immunotherapy response in liver metastasis. Sci Immunol 5, (2020).

22. M. Yadav et al., Predicting immunogenic tumour mutations by combining mass spectrometry and exome sequencing. Nature 515, 572-576 (2014).

23. K. N. Kodumudi et al., Immune Checkpoint Blockade to Improve Tumor Infiltrating Lymphocytes for Adoptive Cell Therapy. PLoS One 11, e0153053 (2016).

24. H. Tang et al., PD-L1 on host cells is essential for PD-L1 blockade-mediated tumor regression. J Clin Invest 128, 580-588 (2018).

25. M. Efremova et al., Targeting immune checkpoints potentiates immunoediting and changes the dynamics of tumor evolution. Nat Commun 9, 32 (2018).

26. C. Tabula Muris et al., Single-cell transcriptomics of 20 mouse organs creates a Tabula Muris. Nature 562, 367-372 (2018).

27. J. Ding et al., Systematic comparative analysis of single cell RNA-sequencing methods. bioRxiv, 632216 (2019).

28. M. J. C. Jordao et al., Single-cell profiling identifies myeloid cell subsets with distinct fates during neuroinflammation. Science 363, (2019).

29. S. V. Kim et al., Modulation of cell adhesion and motility in the immune system by Myolf. Science 314, 136-139 (2006).

30. X. Yu et al., The Cytokine TGF-beta Promotes the Development and Homeostasis of Alveolar Macrophages. Immunity 47, 903-912 e904 (2017).

31. H. Helgeland et al., Transcriptome profiling of human thymic CD4+ and CD8+ T cells compared to primary peripheral T cells. BMC Genomics 21, 350 (2020).

32. O. J. Harrison et al., Epithelial-derived IL-18 regulates Th17 cell differentiation and Foxp3(+) Treg cell function in the intestine. Mucosal Immunol 8, 1226-1236 (2015).

33. N. Isakov, A. Altman, PKC-theta-mediated signal delivery from the TCR/CD28 surface receptors. Front Immunol 3, 273 (2012).

34. L. E. Oikari et al., Cell surface heparan sulfate proteoglycans as novel markers of human neural stem cell fate determination. Stem Cell Res 16, 92-104 (2016).

35. D. Fritz, B. Stefanovic, RNA-binding protein RBMS3 is expressed in activated hepatic stellate cells and liver fibrosis and increases expression of transcription factor Prx1. J Mol Biol 371, 585-595 (2007).

36. M. Riku et al., Down-regulation of the zinc-finger homeobox protein TSHZ2 releases GLI1 from the nuclear repressor complex to restore its transcriptional activity during mammary tumorigenesis. Oncotarget 7, 5690-5701 (2016).

37. H. Kalyanaraman, N. Schall, R. B. Pilz, Nitric oxide and cyclic GMP functions in bone. Nitric Oxide 76, 62-70 (2018).

38. N. Schall et al., Protein kinase G1 regulates bone regeneration and rescues diabetic fracture healing. JCI Insight 5, (2020).

39. J. Baboo et al., The Impact of Varying Cooling and Thawing Rates on the Quality of Cryopreserved Human Peripheral Blood T Cells. Sci Rep 9, 3417 (2019).

40. Q. Wang, T. Li, W. Wu, G. Ding, Interplay between mesenchymal stem cell and tumor and potential application. Hum Cell 33, 444-458 (2020).

41. K. Van den Berge et al., Trajectory-based differential expression analysis for single-cell sequencing data. Nat Commun 11, 1201 (2020).

42. J. Soikkeli et al., Metastatic outgrowth encompasses COL-I, FN1, and POSTN up-regulation and assembly to fibrillar networks regulating cell adhesion, migration, and growth. Am J Pathol 177, 387-403 (2010).

43. Y. Wang, H. Xu, B. Zhu, Z. Qiu, Z. Lin, Systematic identification of the key candidate genes in breast cancer stroma. Cell Mol Biol Lett 23, 44 (2018).

44. J. Li et al., Stromal microenvironment promoted infiltration in esophageal adenocarcinoma and squamous cell carcinoma: a multi-cohort gene-based analysis. Sci Rep 10, 18589 (2020).

45. Y. Gao, S. P. Yin, X. S. Xie, D. D. Xu, W. D. Du, The relationship between stromal cell derived SPARC in human gastric cancer tissue and its clinicopathologic significance. Oncotarget 8, 86240-86252 (2017).

46. A. Escudero-Esparza et al., Complement inhibitor CSMD1 acts as tumor suppressor in human breast cancer. Oncotarget 7, 76920-76933 (2016).

47. S. Ropero et al., Epigenetic loss of the familial tumor-suppressor gene exostosin-1 (EXT1) disrupts heparan sulfate synthesis in cancer cells. Hum Mol Genet 13, 2753-2765 (2004).

48. C. Hafemeister, R. Satija, Normalization and variance stabilization of single-cell RNA-seq data using regularized negative binomial regression. Genome Biol 20, 296 (2019).

49. R. Gaujoux, C. Seoighe, A flexible R package for nonnegative matrix factorization. BMC Bioinformatics 11, 367 (2010).

50. E. Eden, R. Navon, I. Steinfeld, D. Lipson, Z. Yakhini, GOrilla: a tool for discovery and visualization of enriched GO terms in ranked gene lists. BMC Bioinformatics 10, 48 (2009).

51. P. Carmona-Saez, R. D. Pascual-Marqui, F. Tirado, J. M. Carazo, A. Pascual-Montano, Biclustering of gene expression data by Non-smooth Non-negative Matrix Factorization. BMC Bioinformatics 7, 78 (2006).

52. C. Giesen et al., Highly multiplexed imaging of tumor tissues with subcellular resolution by mass cytometry. NatMethods 11, 417-422 (2014).

53. Y. Goltsev et al., Deep Profiling of Mouse Splenic Architecture with CODEX Multiplexed Imaging. Cell 174, 968-981 e915 (2018).

1-168. (canceled)

169. A system comprising one or a plurality of arrays, each array comprising one or a plurality of microwells, each microwell occupying a distinct position on the array and comprising a spatial index primer comprising a nucleic acid molecule comprising, in 5′ to 3′ orientation:
i) an annealing domain comprising a nucleotide sequence that is recognized by a first sequencing primer; and
ii) a spatial barcode domain comprising a nucleotide sequence that is unique to each microwell.

170. The system of claim 169, wherein each array comprises at least about 96, 192, 384 or 768 wells.

171. The system of claim 169, wherein each microwell in the array is from about 50 to about 500 microns in depth.

172. The system of claim 169, wherein the microwells in the array are from about 50 microns to about 500 microns center-to-center spaced.

173. The system of claim 169, wherein the cellular index primer comprising a nucleic acid molecule comprising, from 5′ to 3′:
i) an annealing domain comprising a nucleotide sequence that is recognized by a second sequencing primer; and/or
ii) a capture domain comprising a polythymidine sequence.

174. The system of claim 169, wherein about 10 to about 100 cells are sorted into each well of the multiwell plate.

175. A method of quantifying gene expression in a tissue sample on a single cell level comprising:
a) dividing a sample into at least a first and second subsamples, each subsample comprising at least one messenger RNA (mRNA) from a cell present in the subsample and each subsample corresponding to at least one spatial position of the cell relative to other cells in the sample;
b) positioning each subsample into a microwell occupying a distinct position on an array, each microwell comprising a spatial index primer comprising a nucleic acid molecule comprising, in 5′ to 3′ orientation:
i) an annealing domain comprising a nucleotide sequence that is recognized by a first sequencing primer;
ii) a spatial barcode domain comprising a nucleotide sequence that is unique to each microwell; and
iii) a capture domain comprising a polythymidine sequence;
c) allowing a time period to elapse in physiologically acceptable conditions, the time period sufficient to allow hybridization of the at least one message RNAs (mRNAs) present in each subsample to the capture domain of each spatial index primer;
d) performing reverse transcription to generate cDNA molecules corresponding to the at least one mRNA corresponding to each microwell;
e) pooling cells present in each microwell of the array and sorting into a multiwell plate comprising a plurality of wells;
f) performing an amplification reaction with a cellular index primer to generate reaction products, wherein the cellular index primer comprises a nucleic acid molecule comprising, from 5′ to 3′:
i) an annealing domain comprising a nucleotide sequence that is recognized by a second sequencing primer; and
ii) a cellular barcode domain comprising a nucleotide sequence that is unique to each well of the multiwell plate;
g) sequencing the reaction products obtained in step (f) using the first sequencing primer and the second sequencing primer; and
h) detecting the presence of a nucleotide sequence of a given spatial barcode domain and a given cellular barcode domain, or sequences complementary to a given spatial barcode domain and a given cellular barcode domain;
wherein the step of detecting comprises correlating the presence of a particular nucleotide sequence of the spatial barcode domain unique to a given particular microwell of the array, or the sequence complementary thereof, and correlating the presence of a particular nucleotide sequence of the cellular barcode domain, or the sequence complementary thereof, to the distinct position where the subsample is positioned in said particular microwell of the assay.

176. The method of claim 175 further comprising permeabilizing cells comprised in the tissue sample prior to performing the hybridization.

177. The method of claim 175, further comprising imaging the array with the sample overlaid after contacting the array with the sample.

178. The method of claim 175 further comprising lysing the cells after the cells are sorted into the multiwell plate.

179. A method of generating high-resolution spatial positioning of a nucleic acid expression in a cell within a sample comprising:
a) dividing a sample into at least a first and second subsamples, each subsample comprising at least one messenger RNA (mRNA) from a cell present in the subsample and each subsample corresponding to at least one spatial position of the cell relative to other cells in the sample;
b) positioning each subsample into a microwell occupying a distinct position on an array, each microwell comprising a spatial index primer comprising a nucleic acid molecule comprising, in 5′ to 3′ orientation:
i) an annealing domain comprising a nucleotide sequence that is recognized by a first sequencing primer;
ii) a spatial barcode domain comprising a nucleotide sequence that is unique to each microwell; and
iii) a capture domain comprising a polythymidine sequence;
b) allowing a time period to elapse in physiologically acceptable conditions, the time period sufficient to allow hybridization of the at least one message RNAs (mRNAs) present in each subsample to the capture domain of the each spatial index primer;
c) performing reverse transcription to generate cDNA molecules corresponding to the at least one mRNA corresponding to each microwell;
d) pooling cells present in each microwell of the array and sorting into a multiwell plate comprising a plurality of wells;
e) performing an amplification reaction with a cellular index primer to obtain reaction products, wherein the cellular index primer comprises a nucleic acid molecule comprising, from 5′ to 3′:
i) an annealing domain comprising a nucleotide sequence that is recognized by a second sequencing primer; and
ii) a cellular barcode domain comprising a nucleotide sequence that is unique to each well of the multiwell plate;
f) sequencing the reaction products obtained in step e) using the first sequencing primer and the second sequencing primer; and
g) detecting the presence of a nucleotide sequence of a given spatial barcode domain and a given cellular barcode domain, or sequences complementary to a given spatial barcode domain and a given cellular barcode domain,
wherein the presence of a particular nucleotide sequence of the spatial barcode domain unique to a particular microwell of the array, or the sequence complementary thereto, and the presence of a particular nucleotide sequence of the cellular barcode domain, or the sequence complementary thereto, indicates that the cDNA molecule was obtained from the nucleic acid expressed in one single cell comprised in the subsample at the distinct position where the subsample is positioned in said particular microwell of the assay.

180. The method of claim 179, wherein the method further comprises a step of providing an array comprising a plurality of microwells prior to contacting each subsample to each spatial index primer.

181. The method of claim 179 further comprising lysing the cells after the cells are sorted into the multiwell plate.

182. The method of any of claims 179, further comprising generating sequencing libraries from the cDNA molecules generated in step (f) by tagmentation.

183. The method of claim 182 further comprising performing an amplification reaction following tagmentation.

184. The method of claim 179 further comprising a step of determining which genes are expressed in the cell at a particular distinct location of the tissue sample by a method comprising determining the sequences of the cDNA molecules comprising the same nucleotide sequence of a spatial barcode domain, or sequence complementary thereto, and the same nucleotide sequence of a cellular barcode domain, or sequence complementary thereto.

185. The method of claim 179 further comprising correlating the nucleotide sequence of a spatial barcode domain unique to a given particular microwell of the array, or the sequence complementary thereto, present in the cDNA molecules to a position in the tissue sample.

186. The method of claim 179 further comprising correlating the nucleotide sequence of a spatial barcode domain unique to a given particular microwell of the array, or the sequence complementary thereto, present in the cDNA molecules to an image of the tissue sample.

187. The method of claim 179 wherein the sample is from connective tissue, muscle tissue, nervous tissue or epithelial tissue.
